(19) United States
(12) Patent Application Publication
(10) Pub. No.: US 2026/0284107 A1
(43) Pub. Date: Sep. 24, 2026

(54) PATIENT SPECIFIC INDUCED PLURIPOTENT STEM CELL DERIVED MACROPHAGES FOR CELL BASED THERAPY

(71) Applicant: BOARD OF REGENTS, THE UNIVERSITY OF TEXAS SYSTEM, Austin, TX (US)

(72) Inventors: Chibawanye I. ENE, Houston, TX (US); Frederick F. LANG, Houston, TX (US); Sanjay K. SINGH, Houston, TX (US); Maria Teresa S. BERTILACCIO, Houston, TX (US)

(21) Appl. No.: 19/474,926

(22) PCT Filed: Apr. 12, 2024
(86) PCT No.: PCT/US2024/024298

§ 371 Date: Oct. 13, 2025

Related U.S. Application Data
(60) Provisional application No. 63/495,953, filed on Apr. 13, 2023.

(51) Int. Cl. A61K 35/17; A61K 31/522; A61P 35/00; C07K 14/47; C12N 5/0786; C12N 15/113; C12N 15/85

(52) U.S. Cl. CPC A61K 35/17; A61K 31/522; A61P 35/00; C07K 14/4702; C12N 5/0645; C12N 15/113; C12N 15/85; C12N 2310/531; C12N 2501/22; C12N 2501/2303; C12N 2510/00

(57) ABSTRACT

Described herein are compositions, pharmaceutical compositions, methods, methods of administration, and kits related to cellular therapy for cancer, in particular glioblastoma. In certain aspects, cells can be isolated from a patient, reprogrammed using methods as described herein to a pluripotent state, and then differentiated into cells with a macrophage or macrophage-like phenotype. Differentiated (or terminally-differentiated) cells can then be readministered to a patient in need thereof, for example, a patient with a cancer (for example a glioblastoma). In other aspects, reprogrammed cells can be from a patient's blood, plasma, tissue (for example, a periosteum sample), or other cell source. In other aspects, terminally-differentiated cells can also be genetically modified for self-destruction or for the production of exogenous (or enhanced production of endogenous) cytokines, growth factors, or other peptide-based therapeutics.

### CROSS-REFERENCE TO RELATED APPLICATIONS

This application claims priority to and the benefit of U.S. Provisional Patent Application Ser. No. 63/495,953, filed Apr. 13, 2023, the content of which is incorporated herein by this reference as if fully set forth herein.

### SEQUENCE LISTING

The official copy of the sequence listing is submitted electronically via Patent Center as an XML formatted sequence listing with a file named MDA22_120_1424935.xml, created on Apr. 12, 2024, and having a size of 82 kb. The sequence listing contained in this XML formatted document is part of the specification and is herein incorporated by reference in its entirety.

### BACKGROUND

Immunotherapy including checkpoint inhibitors and chimeric antigen receptor (CAR) T-cell therapy has been successful in a subset of cancers such as leukemia respectively. These therapies, however, have failed for other cancers, for example, nervous system cancers such as glioblastoma multiforme (GBM) in clinical trials. Unlike CAR T-cells, genetically modified macrophages (GEMs) may be more effective for solid cancers given their innate ability to traffic to a myeloid enriched tumor micro-environment, persistence in the immunosuppressive tumor micro-environment, resistance to tumor polarizing signals, the lack of antigen-specific anti-tumor strategy which prevents tumor resistance via antigenic drift, phagocytosis capability and antigen presentation to T-cells via major histocompatibility complex (MHC) class I/II. Although GEMs offer considerable advantages over CAR T-cells, generation of GEMs for clinical application remains a technical challenge.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Described herein are compositions and methods relating to patient-specific macrophage therapies. In certain aspects, described herein are methods of generating patient-specific macrophages, comprising reprogramming one or more somatic cells of a sample from a subject to a pluripotent state; culturing the one or more reprogrammed cells; and differentiating the one or more reprogrammed cells to a macrophage-like phenotype, wherein the differentiation progresses without embryoid body formation. The reprogramming can comprise delivering, into the one or more somatic cells, one or more reprogramming expression vectors comprising nucleic acids encoding OCT3/4, SOX2, KFL4, L-MYC, and LIN28, and shRNA against p53. In certain aspects, at least one of the one or more expression vectors can be an episomal expression vector. In certain aspects, the expansion can comprise expanding the one or more reprogrammed cells for about 10 passages to about 25 passages. In certain aspects, the differentiation can comprise removing b-FGF from the culture media and gradually adding IL-3 and M-CSF over a period of time. In certain aspects, the IL-3 can be added at a concentration of about 20 ng/ml IL-3 to about 30 ng/ml. In certain aspects, the M-CSF can be added at a concentration of about 45 ng/ml to about 55 ng/ml. In certain aspects, the period of time can be about 15 days to about 20 days. In certain aspects, subject can be a subject having, or suspected of having, a glioblastoma. In certain aspects, the sample can comprise pericranium (also referred to herein as “periosteum”) from the subject. In certain aspects, the sample can be enriched for periosteum-derived precursor cells (PDPCs). In certain aspects, the methods can comprise, before reprogramming, mincing the sample after sample collection; culturing the minced sample for a period of time; and isolating one or more somatic cells from the minced sample. In certain aspects, methods as described herein can further comprise, after differentiating, delivering one or more treatment expression vectors to the one or more differentiated cells, the one or more treatment expression vectors comprising a therapeutic nucleic acid, each therapeutic nucleic acid independently encoding a cytokine, a checkpoint protein, or a self-destruction protein. In certain aspects, the cytokine can be interleukin-12 (IL-12), IFN-gamma, or IL-15, individually or in any combination thereof. In certain aspects, the cytokine is interleukin-12 (IL-12). In certains aspects, the checkpoint protein can comprise one or more of PD-1/PD-L1 and CTLA-4/B7-1/B7-2. In certain aspects, the self-destruction protein can be a thymidine kinase. In certain aspects, the expressing vector can also comprise a nucleic acid encoding a protein that can be used as a marker to track cells after transplantation, for example, enhanced green fluorescent protein or CD-19. In certain aspects, the expression vectors can be inducible vectors that only express the cytokine, checkpoint protein, or self-destruction protein in the presence of an inducing agent (such as a doxycycline inducible system, for example).

Also described herein are engineered cells and pharmaceutical compositions comprising cells generated by methods as described herein.

Also described herein are methods of treating a subject in need thereof. In certain aspects, a method of treating a subject in need thereof, comprises administering, to a subject in need of, one or more differentiated cells as described herein to a subject in need thereof. In certain aspects, the subject in need thereof is a subject having or suspected of having a glioblastoma, a neuroblastoma, or a melanoma. In certain aspects, the subject in need thereof can be a subject having or suspected of having a glioblastoma. In certain aspects, the administering can be intra-venous or intra-arterial. In certain aspects, administering is intra-arterial.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic of an aspect of personalized cell-based therapy according to the present disclosure, depicting embodiments of (1) reprogramming; (2) pre-clinical validation studies (flow cytometry, immunohistochemistry) and (3) clinical administration of MDA Hi-Macs.

FIGS. 2A-2B are flow cytometry data showing that reprogrammed macrophages from induced pluripotent stem cells (i.e., HiMacs) express standard macrophage markers CD68, and show a very low incidence of pro-tumor markers (CD206+ and CD163+@ 0.07% of total population). We characterized the immune-phenotype of macrophages derived from the hiPSC lineby multi-color flow cytometry (Fortessa X-20). Live/Dead Fixable Aqua staining was first performed to allow the discrimination of Live/Dead cells. CD14⁺CD68⁺ macrophages were identified by a negative exclusion gating strategy previously described by our group. CD66b⁺ neutrophils were excluded, then, using a lineage (Lin) cocktail including mAbs to CD3, CD19, CD20, and CD56, T cells, B cells, and NK cells were excluded, respectively (>60% Lin−; FIG. 2A,). Further characterization revealed that up to 75% of Lin-cells expressed CD68⁺, a pan macrophage/phagocyte marker (FIG. 2A) Over 97% of the CD68⁺ macrophages were predominantly M1 pro-inflammatory/anti-tumor macrophages as they lacked the classic pro-tumor M2 macrophage markers CD206⁻CD163⁻ (FIG. 2B).

FIGS. 3A-3B are photographs demonstrating that HiMacs according to the present disclosure are amenable to viral transduction (for example, adenovirus transduction by a delta-24RGD oncolytic virus expressing GFP). Anti-hexon staining also performed to indicate viral replication within the Hi-Macs.

FIG. 4 illustrates embodiments of expression cassettes that can be introduced into HiMacs. Constructs such as these allow for the expression of cytokines (such as IL-12) by a HiMac (once introduced into the cell by a method such as transfection, transduction, nucleofection, and the like), as well as a “kill-switch” (CD163 or CD206 self-driven HSV/TK kill switch in an embodiment, for example, SEQ ID NO: 5) that cause the cells to self-destruct before evolving to a pro-tumorigenic phenotype, and can be monitored for current FDA guidelines (<5 copies).

FIG. 5 is a 5× phase contrast micrograph of pericranium progenitor cells expanded from the pericranium of a brain tumor patient undergoing surgery for resection of a glioblastoma.

FIG. 6 depicts a 1ST Generation IL-12 construct with Dox inducible IL-12 expression, as well as CD19t and HSV-TK expression (top panel). Also shown are ELISA (bottom left panel) and western blot (bottom right panel) results for IL-12 expressed only after Dox treatment in HEK 293 T-cells.

FIG. 7 illustrates schematics of the 2^{nd} generation of IL-12 constructs with CD19t and HSV/TK constitutively expressed (with neomycin resistance gene, top panel, for example SEQ ID NOs: 18 and 20, for mouse and human CD19t, respectively) and a separate construct with Doxycycline (Dox) inducible IL-12 expression (with a puromycin resistance gene, bottom panel, for example, SEQ ID NOs: 19 and 21 for mouse and human IL-12, respectively). The 2^{nd} generation improves on the safety of the 1^{st} generation by allowing for constitutive expression of genes that can be used to identify and eliminate the iMacs if needed, while maintaining IL-12 expression under a controlled fashion with doxycycline to prevent toxicity.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of those certain elements.” As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the present disclosure or features of the claims. See, for example, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20. Exemplary vector backbones, in addition to other vector features (i.e., kozak sequence (e.g., GACACC), pre-coding sequences, signal peptides, cleavage domains (e.g., P2A domain), linkers, secretion signals, promoters, and inducible systems can be found at least in the informal sequence table on the last pages of the present application.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected or transduced gene (“transduced” meaning a gene introduced into a cell by a viral vector, such as infection by a lentivirus) can occur transiently or stably in a cell. During “transient expression” the transfected or transduced gene is not transferred to the daughter cell during cell division. Since its expression is restricted to the transfected or transduced cell, expression of the gene is lost over time. In contrast, “stable” expression of a transfected or transduced gene can occur when the gene is co-transfected with another gene that confers a selection advantage to the transfected or transduced cell. Such a selection advantage may be a resistance towards a certain toxin that is presented to the cell. Expression of a transfected or transduced gene can further be accomplished by transposon-mediated insertion into to the host genome. During transposon-mediated insertion, the gene is positioned in a predictable manner between two transposon linker sequences that allow insertion into the host genome as well as subsequent excision. “Inducible” expression means a transfected or transduced gene is only expressed under certain conditions and can be turned on and off, for example, in the presence of a transducing agent such as tetracycline or doxycycline.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The term “vector” refers to a carrier DNA molecule into which a DNA sequence can be inserted for introduction into a host cell. In some embodiments, vectors of use according to the present disclosure are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector, e.g., a promoter. Vectors include non-viral vectors such as plasmids and viral vectors.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “operably linked” refers to a functional linkage between a first nucleic acid sequence and a second nucleic acid sequence, such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

“Expression cassette” refers to a polynucleotide comprising a promoter or other regulatory sequence operably linked to a sequence encoding a protein.

The term “siRNA” refers to a nucleic acid that forms a double stranded RNA, which double stranded RNA has the ability to reduce or inhibit expression of a gene or target gene when the siRNA expressed in the same cell as the gene or target gene. In the context of the present disclosure, the term “siRNA” includes miRNA. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The term “shRNA” refers generally to an siRNA that is introduced into a cell as part of a larger DNA construct. Typically, such constructs allow stable expression of the siRNA in cells after introduction, e.g., by integration of the construct into the host genome.

An “antisense” oligonucleotide or polynucleotide is a nucleotide sequence that is substantially complementary to a target polynucleotide or a portion thereof and has the ability to specifically hybridize to the target polynucleotide.

Ribozymes are enzymatic RNA molecules capable of catalyzing specific cleavage of RNA. The composition of ribozyme molecules preferably includes one or more sequences complementary to a target mRNA, and the well-known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “a recombination of amino acid sequences,” in the context of a peptide, refers to a change or variation in the amino acid sequence of a reference peptide, such that the biological properties of the reference peptide are maintained after the amino acid sequence change. For example, the recombination of amino acid sequence may be a conservative amino acid substitution or an amino acid sequence modification (addition, deletion or substitution) to produce a chimeric peptide.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20

The term “library” is used according to its common usage in the art, to denote a collection of molecules, optionally organized and/or cataloged in such a way that individual members can be identified. Libraries can include, but are not limited to, combinatorial chemical libraries, natural products libraries, and peptide libraries.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

The term “allogeneic,” in the context a cell, refers to a donor cell that is introduced to a recipient that is not genetically identical to the donor.

The term “autologous,” in the context a cell, refers to a donor cell that is introduced to a recipient that is genetically identical to the donor.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

The term “marker” in the context of a cell or tissue (e.g. a normal or cancer cell or cancer stem cell) means any gene product, e.g., non-coding RNA (non-messenger RNA), mRNA and polypeptide, antigen, molecule or other chemical or biological entity that is specifically found in or on a cell of interest and can be used to identify the cell affected by a disease or disorder.

The term “expression level” when referring to a cell marker such as a leukemia stem cell marker, refers to the measurable quantity of a gene product produced by the gene in a sample of a patient wherein the gene product can be a transcriptional product or a translated transcriptional product. Accordingly, the expression level can pertain to a nucleic acid gene product such as RNA or cDNA or a polypeptide. The expression level is derived from a biological sample, a subject's sample, a cell culture sample, and/or a control sample, and can for instance be detected de novo or correspond to a previous determination.

The term “detecting an expression level” or “expression level is detected” as used in reference to a gene means the application of a method to a sample, e.g., a subject sample, a biological sample, a cell culture sample, and a control sample, for ascertaining quantitatively, semi-quantitatively or qualitatively the amount of a gene expression product, e.g., RNA, mRNA or polypeptide product. For example, a level of a gene expression can be determined by a number of methods including, but not limited to, arrays and other hybridization based methods and PCR protocols. In some instances, the PCR methods include a probe or primer or primer set that are used to ascertain the amount of nucleic acid of the gene. For example, an expression level of a gene can be determined using a probeset or one or more probes of the probeset, described herein for a particular gene. In addition more than one probeset where more than one exists, can be used to determine the expression level of the gene.

Other examples of methods for detecting the amount of gene expression product present in a sample include Nanostring® technology, serial analysis of gene expression (SAGE), RNA sequencing, RNase protection assays, and Northern Blot. The polypeptide level can be determined by an immunoassay, such as western blot, flow cytometry, immunohistochemistry, ELISA, immunoprecipitation and the like, where a gene or gene signature detection agent such as an antibody, for example, a labeled antibody specifically binds the polypeptide product encoded by the gene and the relative or absolute amount of polypeptide in a sample can be ascertained.

The term “isolated,” when applied to a protein, denotes that the protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. Particularly, it means that the protein is at least 85% pure, more preferably at least 95% pure, and most preferably at least 99% pure.

“Contacting” is used in accordance with its plain ordinary meaning and refers to the process of allowing at least two distinct species (e.g. chemical compounds including biomolecules, or cells) to become sufficiently proximal to react, interact or physically touch. The two species may be a cell (e.g., a transfected non-totipotent cell, a non-totipotent cell, a totipotent cell) as described herein and an inhibitor (e.g., zygote-specific gene repressor inhibitor, growth factors) as described herein. In some embodiments, contacting may involve a transfected non-totipotent cell as described herein and a zygote-specific gene repressor inhibitor. In other embodiments, contacting may involve a non-totipotent cell as described herein and a zygote-specific gene repressor inhibitor. It should be appreciated, however, that the resulting reaction product can be produced directly from a reaction between the added reagents or from an intermediate from one or more of the added reagents which can be produced in the reaction mixture.

A “somatic cell” as used herein refers to differentiated, or partially differentiated cells relative to embryonic stem cells. Thus, the term includes, e.g., cells such as fibroblasts that are derived from embryonic stem cells, but are differentiated. Somatic cells include cells making up organs, skin, blood, bones and connective tissue in an organism, but not germline cells.

A “primary cell” is a cell taken directly from living tissue (e.g., via biopsy) and is established for growth in vitro. Such cells may be representative of the main function component of the tissue from which they are derived. Primary cell types include but are not limited to fibroblasts, including mouse embryonic fibroblasts (MEF), keratinocytes, melanocytes, myoblasts, mesenchymal cells endothelial cells, epithelial cells, fat cells and stromal cells.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

The term “reprogramming” refers to the process of dedifferentiating a non-pluripotent or a non-totipotent cell (typically a somatic cell) into a cell exhibiting pluripotent or totipotent stem cell characteristics.

“Self-renewal” refers to the ability of a cell to divide and generate at least one daughter cell with the self-renewing characteristics of the parent cell. The second daughter cell may commit to a particular differentiation pathway. For example, a self-renewing hematopoietic stem cell can divide and form one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Non-self-renewing cells refer to cells that undergo cell division to produce daughter cells, neither of which have the differentiation potential of the parent cell type, but instead generate differentiated daughter cells.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, for example, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

An “induced pluripotent stem cell” refers to a pluripotent stem cell artificially derived from a non-pluripotent cell. A non-pluripotent cell can be a cell of lesser potency to self-renew and differentiate than a pluripotent stem cell. Cells of lesser potency can be, but are not limited to somatic stem cells, tissue specific progenitor cells, primary or secondary cells.

The term “embryonic stem cell” is used to refer to the pluripotent stem cells of the inner cell mass of the embryonic blastocyst (see U.S. Pat. Nos. 5,843,780, 6,200,806). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Illustrative distinguishing embryonic stem cell characteristics include, without limitation, gene expression profile, proliferative capacity, differentiation capacity, normal karyotype, responsiveness to particular culture conditions, and the like.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “isolated cell” as used herein refers to a cell that has been removed from an organism in which it was originally found or a descendant of such a cell. An “isolated” cell may be cultured in vitro in the presence of other cells. Optionally, the cell is later introduced into a second organism or re-introduced into the organism from which it (or the cell from which it is descended) was isolated.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include, Oct-1, Oct-2, Oct-3/4, Oct-6, Oct-7, Oct-8, Oct-9, and Oct-11. e.g. Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, or 95% amino acid sequence identity across their whole sequence compared to a naturally occurring Oct polypeptide family member such as to those listed above or such as listed in Genbank accession number NP_002692.2 (human Oct4) or NP_038661.1 (mouse Oct4). Oct polypeptides (e.g., Oct3/4) can be from human, mouse, rat, bovine, porcine, or other animals. Generally, the same species of protein will be used with the species of cells being manipulated.

A “Klf polypeptide” or a “KLF” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J. & Yang, V. W., Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. See, Nakagawa, et al., Nature Biotechnology 26:101-106 (2007). In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated.

A “Myc polypeptide” or a “Myc” refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated.

A “Sox polypeptide” or a “Sox” refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, e.g., Dang, D. T., et al., Int. J. Biochem. Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. See, Nakagawa, et al., Nature Biotechnology 26:101-106 (2007). In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated.

The term “p53” refers generally to a protein of a molecular weight of about 55 kDa on SDS PAGE that functions as a tumor suppressor. The protein and nucleic sequences of the p53 protein from a variety of organisms from humans to Drosophila are known and are available in public databases, such as in accession numbers, NM_000546, NP_000537, NM_011640, and NP_035770, for the human and mouse sequences. Mammalian p53 sequences are highly conserved between species. Mouse and human p53 proteins are 85% identical. In humans, p53 is encoded by the TP53 gene located on the short arm of chromosome 17 (17p13.1). A p53 protein in the context of the present disclosure, this can includes allelic variants and other functional variants and orthologs. In some embodiments, variants have at least 85%, at least 90%, or at least 95%, or greater, amino acid sequence identity across their whole sequence compared to a naturally occurring p53 family member such as that listed under accession number NP_000537 (human p53). Generally, the same species of protein will be used with the species of cells being manipulated.

The term “LIN28” refers to an RNA-binding protein that binds to and enhances the translation of the IGF-2 (insulin-like growth factor 2) mRNA. The protein and nucleic sequences of the LIN28 protein from a variety of organisms from humans to Drosophila are known and are available in public databases, such as in accession numbers, AAH28566.1, XP_011540450.1, Q8K3Y3.1, and XP_006539380.1, for the human and mouse sequences. A LIN28 protein in the context of the present disclosure, can includes allelic variants and other functional variants and orthologs. In some embodiments, variants have at least 85%, at least 90%, or at least 95%, or greater, amino acid sequence identity across their whole sequence compared to a naturally occurring LIN28 family member such as that listed under accession number AAH28566.1 (human LIN28). In some embodiments, said variants retain the functionality of LIN28 (e.g., at least 60%, 70%, 80%, 90%, or 95% of the activity of LIN28). Generally, the same species of protein will be used with the species of cells being manipulated.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and also includes cells in cell culture, cell supernatants, cell lysates, serum, plasma, biological fluids, and tissue samples.

The terms “individual,” “subject,” “host,” and “patient,” used interchangeably herein and refer to any mammalian subject for whom diagnosis, treatment, or therapy is desired, particularly humans.

The term “normal” as used in the context of “normal cell,” is meant to refer to a cell of an untransformed phenotype or exhibiting a morphology of a non-transformed cell of the tissue type being examined. A “cancer cell” refers to a cell of a cancer that can be identified by abnormalities in, for example, cell growth or proliferation (e.g., uncontrolled growth or proliferation), regulation of the cell cycle, cell mobility, cell-cell interaction, or metastasis, etc.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% (or more) higher.

The terms “administering,” “delivering,” and “introducing,” can be used interchangeably to indicate the introduction of a therapeutic composition or agent (e.g., cells) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a period of time, for example, a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a cancerous malignancy or nervous system disorder. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “cancer,” “neoplasm,” “tumor,” and “carcinoma,” are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Types of cancer that can are related to the present disclosure include, but are not limited to, nervous system malignancies, for example, glioblastomas.

“Glioblastoma” or “glioblastoma multiforme” (also known as a grade IV astrocytoma) refers to a type of low-survival cancer that forms from glial cells in the brain, in particular astrocytes, which provide critical support for neurons for proper neuronal function. They are typically diagnosed in their “stage IV” form and not in a precursor stage. They typically can spread within the CNS (even across the corpus callosum) but do not typically spread outside the CNS. They are difficult to treat and there is currently no cure. Even with aggressive treatment that includes surgery, chemotherapy and radiation therapy, the survival is about 14 months. Therefore there is a desparate need for more effective treatments for GBM. Unlike other cancers such as melanoma, immunotherapy has been largely unsuccessful for GBM. This is due, in part, to the immunosuppressive environment of these tumors. Myeloid cells such as macrophages are the most prevalent immune cell within GBM and contribute significantly to the immunosuppressive environment. This indicates that targeting macrophages either with drugs or administration of decoy macrophages that are immune stimulating such as Hi-Macs could overcome immunosuppression and allow the immune system to be more effective against GBM. Methods and compositions as described herein can be suitable for any solid tumor including neuroblastoma or melanoma.

“Neuroblastoma” refers to a tumor that develops from the sympathetic nervous system, such as the adrenal gland or sympathetic ganglia (Brodeur, Nat. Rev. Cancer, 2003, 3:203-216). It is one of the most frequent solid tumors in children. It is the most common malignancy diagnosed in the first year of life and shows a wide range of clinical phenotypes with some patients having tumors that regress spontaneously, whereas the majority of patients have aggressive metastatic disease (Maris et al., Lancet, 2007, 369:2106-20). These latter neuroblastoma cases have survival probabilities of less than 40% despite intensive chemoradiotherapy, and the disease continues to account for 15% of childhood cancer mortality (Maris et al. (2007) Lancet, 369:2106-20; Matthay et al. (1999) N. Eng. J. Med., 341:1165-73). The cancer can start in neuroblasts (e.g., early nerve cells) of the sympathetic nervous system. The term neuroblastoma includes any stage of the cancer as determined according to, for example, the International Neuroblastoma Staging System (INSS) or the International Neuroblastoma Risk Group Staging System (INRGSS).

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the following terms have the meanings ascribed to them unless specified otherwise. Other terms used in the fields of recombinant nucleic acid technology, microbiology, immunology, and molecular and cell biology as used herein will be generally understood by one of ordinary skill in the applicable arts.

### II. Introduction

Glioblastomas are the worst form and most common type of brain tumors in adults (Stupp, R. et al. Radiotherapy plus concomitant and adjuvant temozolomide for glioblastoma. N Engl J Med 352, 987-996 (2005). https://doi.org: 10.1056/NEJMoa043330). The median survival of 14 months has not changed significantly over the last decade. Immunotherapy including checkpoint inhibitors and chimeric antigen receptor (CAR) T-cell therapy has been successful in a subset of cancers such as leukemia respectively (Tawbi, H. A. et al. Relatlimab and Nivolumab versus Nivolumab in Untreated Advanced Melanoma. N Engl J Med 386, 24-34 (2022). doi.org: 10.1056/NEJMoa2109970; Sheykhhasan, M., Manoochehri, H. & Dama, P. Use of CAR T-cell for acute lymphoblastic leukemia (ALL) treatment: a review study. Cancer Gene Ther 29, 1080-1096 (2022). doi.org: 10.1038/s41417-021-00418-1).

These therapies, however, have failed for glioblastoma (GBM) in clinical trials. This is due in part to the inability of CAR T-cells to migrate throughout the tumor tissue, antigen drift following CAR targeting of specific epitopes and the inability of T-cells to persist in the immunosuppressive solid tumor micro-environment.

Unlike CAR T-cells, genetically modified macrophages (GEMs) may be more effective for solid cancers given their innate ability to traffic to a myeloid enriched tumor micro-environment, persistence in the immunosuppressive tumor micro-environment, resistance to tumor polarizing signals, the lack of antigen-specific anti-tumor strategy which prevents tumor resistance via antigenic drift, phagocytosis capability and antigen presentation to T-cells via major histocompatibility complex (MHC) class I/II. Although GEMs offer considerable advantages over CAR T-cells, generation of GEMs for clinical application remains a technical challenge. The present inventors have generated GEMs from circulating monocytes (Brempelis, K. J. et al. Genetically engineered macrophages persist in solid tumors and locally deliver therapeutic proteins to activate immune responses. J Immunother Cancer 8 (2020). doi.org: 10.1136/jitc-2020-001356; Klichinsky, M. et al. Human chimeric antigen receptor macrophages for cancer immunotherapy. Nat Biotechnol 38, 947-953 (2020). doi.org: 10.1038/s41587-020-0462-y). Given that monocytes make up only 2-8% of total circulating white blood cells and have limited proliferation in-vitro, to obtain enough starting monocytes to generate GEMs, monocyte expansion stimulants (e.g., Filgrastim/Neulasta/recombinant granulocyte-colony stimulating factor or G-CSF) have to be administered to patients in the outpatient setting daily for up to 1 week prior to harvesting monocytes (Klichinsky, M. et al. Human chimeric antigen receptor macrophages for cancer immunotherapy. Nat Biotechnol 38, 947-953 (2020). doi.org: 10.1038/s41587-020-0462-y) or multiple HLA-matched donor monocytes have to be combined.

Although GEMs offer considerable advantages over CAR T-cells, generation of GEMs for clinical application remains a technical challenge. GEMs have been generated from circulating monocytes (Brempelis et al) previously. However, given that monocytes make up only 2-8% of total circulating white blood cells and have limited proliferation in-vitro, to obtain enough starting monocytes to generate GEMs, monocyte expansion stimulants (Klichinsky et al; Filgrastim/Neulasta/recombinant granulocyte-colony stimulating factor or G-CSF) have to be administered to patients in the outpatient setting daily for up to 1 week prior to harvesting monocytes or multiple HLA-matched donor monocytes have to be combined.

Here, deriving clinically relevant numbers of GEMs from patient specific induced pluripotent stem cells is described. This obviates the need for pooling of monocytes or need for administration of expensive monocyte stimulating factors.

Administering a stimulant prior to harvesting circulating monocytes for macrophage differentiation is cost prohibitive. Neulasta®, for example, is $6,417 per dose, daily for 1 week and may be associated with side effects including spleen rupture, acute respiratory distress syndrome, kidney injury, thrombocytopenia, and myelodysplastic syndrome or leukemia formation.

Several reports demonstrate that cancer alters the transcriptome of human circulating monocytes. Cassetta L. et al. demonstrated that human endometrial and breast cancer severely modify the transcriptome of circulating monocytes and their progeny macrophages (Cassetta, L. et al., 2019, Cancer Cell 2019, 35 (4): 588-602.E10, doi.org/10.1016/j.ccell.2019.02.009). Therefore, it may not be ideal to derive GEMs from circulating monocytes that have been transcriptionally reprogrammed by the tumor toward a protumor function. This not only impedes therapeutic efficacy of GEMs, but could also make the tumors more aggressive.

The use of donor allogenic sources (HLA matched) is problematic because, like organ transplantation, HLA matched donors must be found, which may be difficult and expensive. Additionally, there is a high risk of rejection by the patient's immune system which could affect treatment efficacy. To minimize systemic rejection, treated patients need to be immunosuppressed prior to receiving HLA matched GEMs which hampers efficacy of immunotherapy or to minimize systemic rejection HLA matched GEMs need to be injected directly into the brain tumor which requires a second surgical procedure.

Autologous human patient specific induced pluripotent stem cell (iPSC) derived GEMs (or ‘iMacs’ or ‘HiMacs’ or ‘hi-Macs’ or ‘MDA hi-Macs’ as used herein) can be generated from the patient's own tissue (e.g., from pericranium tissue, also referred to herein as “periosteum” or “periosteum tissue”) without need for stimulation and can be administered systemically (intra-arterially or intra-venously) with minimal rejection by the patient's immune system. Furthermore, systemic administration of hi-Macs could work better since the vascular delivery (intravenous or intra-arterial) allows for more diffuse homing to regions of infiltrative tumor that rely on vascular supply. This systemic delivery approach also makes the use of hi-iMacs applicable to other types of cancers.

### Commercial Aspects of the Present Example

Deriving MDA hi-Macs with high yield according to the present disclosure obviates the need for administration of expensive recombinant stimulants. High yield is evident because stem cell sources for the macrophages exist that can produce macrophages for over 4 weeks (instead of patients where the source is limited).

MDA hi-Macs can be infused systemically with lower chances of immune rejection. According to some embodiments of the present disclosure, MDA hi-macs can be administered via intra-arterial direct delivery or intravenous systemic delivery. For either delivery, MDA hi-macs can be resuspended in plasmalyte supplemented with 0.5% human serum albumin (HSA or other isotonic solution). For the intra-arterial delivery, MDA hi-Macs can be resuspended in 20 mls of plasmalyte with HSA. The entire volume can be administered at 1 ml per min into a tumor feeding vessel that is accessed in the angiography suite. For intravenous delivery, MDA hi-Macs can be resuspended in 100 mls of plasmalyte with HSA and administered via IV access. Therefore, there is no need for a repeat brain surgery to deliver the agents directly to the tumor.

Donor GEMs require going through expensive HLA-matching. HLA-matching is not needed for MDA hi-Macs generation or administration back to the patient.

### Aspects of the Cell Line Generation According to the Present Disclosure

Hi-Macs can be derived from any cell type in the patient (EX. Blood cells or skin fibroblast) that can be re-programmed into iPSCs. However, a source that has a resident stem-like cell population would enhance the reprogramming efficiency to IPSCs. Described herein is a protocol for harvesting pericranium (the tissue overlying the skull) that is enriched with stem-like multipotent periosteum derived precursor cells (PDPCs). In some embodiments of the present disclosure, to derive PDPCs, pericranium can be collected from the skull of patients undergoing brain tumor surgery. The collected pericranium can be rinsed with Hanks solution, mechanically minced and digested for 2 hours in DMEM/Ham's F12 medium containing collagenase, human serum and antibiotics. Cells can be pelleted and resuspended in DMEM/Hams F12 medium with human serum and plated on dishes to allow for attachment over 1 week. Attached cells containing PDPCs (appearing fibroblast-like in shape) can then be collected from the dish and re-programed into iPSCs. Unlike blood cells or skin fibroblasts which have mainly terminally differentiated cells, periosteum is enriched with PDPCs that can more efficiently reprogram into iPSC and subsequently macrophages. Therefore, methods as described herein are superior to other sources given that a stem cell like source (PDPCs) for the iPSCs is used. Giving expensive stimulants that are required for a peripheral blood source for macrophages is also avoided according to methods of the present disclosure (i.e., no additional stimulants are given to the patient for the purpose of macrophage stimulation).

In certain aspects, pericranium for generation of MDA hi-Macs is described when patients are undergoing their initial surgery for brain tumors (an IRB approved protocol; 2022-0943 “Induced pluripotent stem cell derived genetically modified macrophages (HiMacs) for personalized cell-based cancer therapy). In some embodiments, a 2 cm×2 cm pericranium flap can be harvested during brain tumor surgery and sent to the lab for deriving PDPCs and subsequentily reprograming to iPSC.

While the patient is undergoing standard of care and follow-up imaging, HiMacs can be derived and stored for treatment of the patient's disease recurrence, which is inevitable with a disease like GBM. In some embodiments, Hi-Macs can be stored in standard freezing medium containing containing 10% DMSO. Prior to use, these cells can be washed three times in PBS or plasmalyte prior to further studies or clinical administration.

### Differentiation of Present Methods Over the Literature

Brempelis et. al.: Circulating monocytes were used for this pre-clinical study but it required pooling of HLA matched monocytes from multiple donors or use of autologous monocytes derived from patients receiving stimulants. Protocols as described herein are distinct, as peri-osteum (or blood) can be used to generate iPSCs then hi-Macs. In some embodiments according to the present disclosure, to derive PDPCs, pericranium can be collected from the skull of patients undergoing brain tumor surgery. The pericranium can be rinsed with Hanks solution, mechanically minced and digested for 2 hours in DMEM/Ham's F12 medium containing collagenase, human serum and antibiotics. Cells can be pelleted and resuspended in DMEM/Hams F12 medium with human serum and plated on dishes to allow for attachment over 1 week. Attached cells containing PDPCs can be collected from the dish and re-programed into iPSCs. Furthermore, the differentiation steps for hi-Macs require a different set of cytokines from Brempelis et. al., and is differentiated from other methods (such as Ackermann et al. below), in part, because the methods as described herein are modified to shorten the time for MDA hi-Macs derivation. MDA hi-Macs will be personalized for the patient and will not require stimulants to be administered.

Ackermann et. al.: This group published a differentiation protocol for iPSCs to macrophages which takes up to 4 weeks. This protocol has been modified, for example, (with similar yield) by skipping embryoid body formation, terminal differentiation, and the need for bioreactor for mass production. This allows for a 2-week time from iPSC to MDA hi-Macs allowing for a more expedited delivery to patients in need thereof. Therefore, protocols as described herein, MDA hi-Macs can be generated and administered back to patients in a shorter time frame according to the present disclosure compared to the work of other groups.

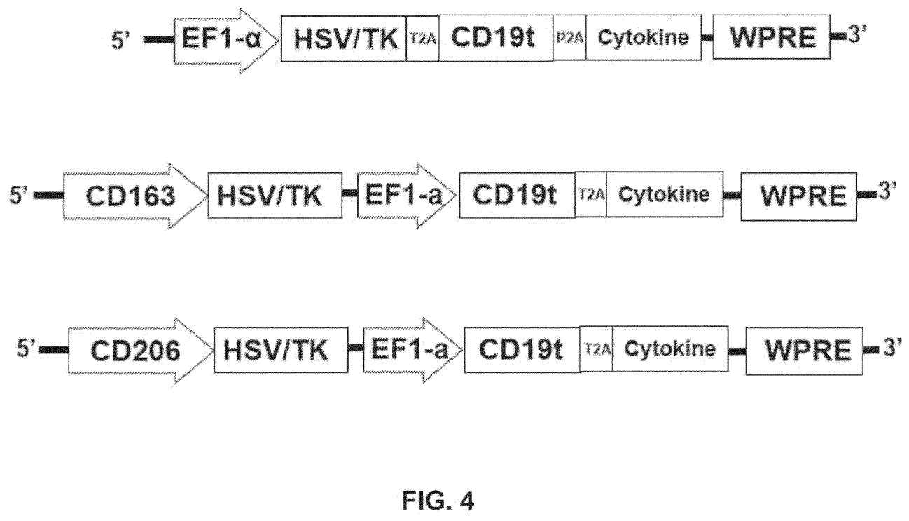

Given reports of patient circulating monocyte derived GEMs that have the tendency to revert to protumor phenotype following administration, a genetic switch can be introduced that induces self-destruction if the MDA hi-Macs become a pro-tumor phenotype (FIG. 4). This approach has not been described and would ensure the safety of the product following administration

To overcome such obstacles and challenges, described herein utilization of patient-specific induced pluripotent stem cell (iPSCs)-derived macrophages (HiMacs). Described herein are methods of production; compositions including such; methods of administration; engineered cells related to such; and the like. Furthermore, described herein are also modified cells for delivery of novel therapeutics such as IL-12, IFN-γ (for example NCBI Accession: AAB59534.1), IL-15 (for example, NCBI Accession: AAI00963.1), MCP-1 (for example, Accession: AAB29926.1), and Delta-24RGD oncolytic adenovirus back to the patient from which the iPSC was derived. This allows for generation of enough cells for clinical trials, multiple treatments for the same patient while minimizing immune mediated rejection of the GEM.

Various methods in the art can be used to modify cells for delivery of therapeutics. In some embodiments, delivery of therapeutics can comprise delivering one or more nucleic acids encoding IL-12 subunts α and β (for example, human or mouse IL-12 subunits, such as SEQ ID NO: 8 and 10 for mouse IL-12β and IL-12α, respectively, and SEQ ID NOs: 16 and 17 for human IL-12β and IL-12α, respectively). Nucleic acids can be delivered by methods known in the art, such as transfection, electroporation, or viral transduction (e.g., by a lentivirus or adeno-associated virus) of vectors (e.g., plasmids) that express coding sequences of a protein of interest. In some embodiments, such coding sequences can be operably linked to a constitituve promoter (e.g., CAG or elongation factor 1α (EF1α)). In some embodiments, such coding sequences can be operably linked to an inducible promoter or other inducible element (such as a repressor that requires removal for transcription) that requires the presence of an inducing agent (i.e., tetracycline or doxycycline, among others). In some embodiments, the nucleic acid can be packaged into a lentivirus that can be used to transduce cells as described herein. Exemplary vector systems that can be used include the backbones of SEQ ID NOs: 6 and 11, for example. Exemplary IL-12-expressing constructs include SEQ ID NOs: 19 and 21.

Methods and compositions according to the present disclosure can provide a source for cell-based therapy that can overcome these problems associated with CAR-T cells. MDA-HiMacs, for example, can also deliver genes, cytokines, viruses, perform phagocytosis and present antigens (the latter 3 cannot be performed by CAR T-cells). Other sources of macrophages such as monocytes require pooling of donor samples or preharvest stimulation in patients to get enough cell numbers for clinical application. These strategies require expensive drugs administered prior to monocyte harvest. Approaches as described herein also overcomes these hurdles by generating a high yield of macrophages for clinical applications without pre-treatment, in particular the administration of additional cellular stimulants to the patient before a sample is collected for cellular isolation, reprogramming, and differentiation.

MDA-HiMacs could be used to deliver immune stimulatory cytokines, full length checkpoint antibodies, oncolytic viruses, exosomes, mRNA, tumor antigens to any cancer type. MDA-HiMacs could be used to study how macrophages can change the tumor micro-environment to an immunosuppressive micro-environment. This will allow for uncovering mechanisms to overcome this phenomenon in patients. MDA-HiMacs could be used to study basic functions of macrophages in non-cancer disease states like inflammation and infection.

Currently CAR-T cells are FDA approved only as cell-based immune therapy for cancer. CAR T-cells have been effective for many liquid tumors, but most solid cancers have been resistant to therapy. This is due, in part, to the inability of T-cells to migrate through solid tumors or persist in the macrophage-driven immunosuppressive micro-environment.

The present disclosure relates to treatment of cancerous malignancies, in particular, solid tumors for which CAR T-cell therapy is ineffective (in particular glioblastoma, also referred to herein as glioblastoma multiform or GBM) using the MDA-HiMacs as described in this disclosure. Any cancer comprising solid tumors could benefit from the compositions and methods as described herein. Additional examples include, without intending to be limiting, melanoma, breast cancer, and ovarian cancer.

### III. In Vitro Methods

Described herein are methods of reprogramming pluripotent cells to macrophages (or cells with a macrophage-like phenotype displaying pro-macrophagic markers with the absence of non-macrophagic markers).

In certain aspects, described herein are somatic or precursor cells (or populations of such) that can be reprogrammed into induced pluripotent stem cells (iPSCs). Such cells can come from a patient sample of a patient diagnosed or suspected of having a cancerous malignancy of the present disclosure (i.e., an autologous cell source), or from another subject, for example an HLA-matched subject to the patient diagnosed or suspected of having a cancerous malignancy (i.e., an allogenic source).

Patient samples can be blood, saliva, plasma, urine, tissue, cerebro-spinal fluid (CSF), or another sample type. In certain aspects, the tissue may be periosteum. In certain aspects of methods as described herein, patient samples are collected without providing the patient or subject any cellular stimulants.

Cells for reprogramming can be isolated or otherwise enriched from the patient sample prior to reprogramming. In some embodiments, to derive iPSCs from pericranium, 2 cm×2 cm pericranium (or another size, for example, 1 cm×1 cm) can be collected from the skull of patients undergoing brain tumor surgery. The pericranium can be rinsed with Hanks solution, mechanically minced and digested for 2 hours in DMEM/Ham's F12 medium containing collagenase, human serum and antibiotics. Cells can be pelleted and resuspended in DMEM/Hams F12 medium with human serum and plated on dishes to allow for attachment over 1 week. Attached cells containing PDPCs can be collected from the dish and re-programed into iPSCs. In some embodiments, to derive iPSCs from blood, patient blood can be collected and PBMCs isolated using a ficoll-gradient. PBMCs can then be re-programmed into iPSCs.

Following isolation from the patient sample, cells can be reprogrammed to a pluripotent state (i.e., into induced pluripotent stem cells) utilizing establishing protocols. For example, vectors (in particular episomal vectors or vectors that otherwise lack chromosomal integration), can be delivered to cells through known methods (for example, transfection, transduction, nucleofection, and the like) that can over-express reprogramming factors (such as OCT-family proteins, Klf-family proteins, Myc-family proteins, and Sox-family proteins, and LIN-family proteins additionally) while knocking down, knocking out, or otherwise reducing expression of other cell cycle regulator proteins, for example, p53-family proteins. Other reprogramming methods, for example, the use of sendai-virus vectors could also be employed according to the present disclosure.

Following reprogramming of the cells to a pluripotent state, cells can then be differentiated (or terminally-differentiated) into iPSC-derived macrophages (or iPSC-derived cells exhibiting a macrophage-like phenotype and expressing macrophagic markers while lacking expression of non-macrophagic markers that are specific markers for non-macrophagic cells). Such differentiation can be accomplished by culturing the cells in a differentiation mediaover a period of time, that is different than the reprogramming media or the maintenance media used to maintain the cells in a pluripotent state. In some embodiments, first, the media for iPSCs (with approximately 80% confluency) cultured in iPSC media is changed daily with iPSC media+10 μM ROCK inhibitor and 25% reduced concentration bFGF over the span of four consecutive days (about 7.5 ng/ml, about 5 ng/ml, about 2.5 ng/ml, and about 0 ng/ml, respectively). Starting on day 10, media can be gradually switched to X-VIVO-15 (LONZA) media comprising 25 ng/ml of IL3 and 50 ng/ml of M-CSF, where day 10 media composition comprises 75% iPSC media comprising 10 μM ROCK inhibitor can be mixed with 25% X-VIVO-15 media (comprising IL3 and M-CSF), day 11 media can comprise 50% iPSC media comprising 10 μM ROCK inhibitor and can be mixed with 50% X-VIVO-15 media (comprising IL3 and M-CSF), day 12 media can be 25% iPSC media comprising 10 μM ROCK inhibitor is mixed with 75% X-VIVO-15 media (comprising IL3 and M-CSF), and day 13 media can be 100% X-VIVO-15 media (comprising IL3 and M-CSF). Incubations can be carried out on orbital shaker set at about 100 revolutions per minute. Supernatant/conditioned media with hi-MACS can then be collected and terminally differentiated by culturing in media comprising 50 ng/ml of M-CSF for 7 days.

After differentiation of the cells to macrophages (or cells with a macro-phage like phenotype; cells referred to herein as iMacs or hiMacs or hi-macs—MDA-hiMacs), cells can further be phenotype and isolated according to various cellular markers (such as IBA-1 or CD68, for example, and absence of pro-tumor markers, such as CD206 or CD163) if desired.

Such differentiated cells can optionally undergo further genetic differentiation. For example, vectors (such as pLenti-EF1a-C-tGFP, for example) that express or knockdown a gene of interest can be introduced into the cell (for example viral vectors that overexpress a cytokine or growth factor or other therapeutic peptide). Vectors that also make the cell more or less amenable to cell destruction or cell death can be added, if desired (for example, a CD163 promoter-driven HSV/TK switch).

In an embodiment, a method of generating patient-specific macrophages, can comprise reprogramming one or more somatic (or precursor) cells of a sample from a subject to a pluripotent state; and differentiating the one or more reprogrammed cells to a macrophage-like phenotype, wherein the differentiation progresses without embryoid body formation. Such cells must be positive for the markers Iba1 and CD68, and must lack markers for T-cells, NK-cells, dendritic cells and b-cells or pro-tumor markers such as CD163 or CD206. The cells should also lack OCT3/4 expression, which can be verified by immunocytochemistry techniques as known the art. In other embodiments of methods as described herein, methods can further comprise culturing the one or more reprogrammed cells and expanding the one or more reprogrammed cells to a population of induced pluripotent stem cells.

According to some embodiments of the present disclosure, iPSCs can be derived/cultured/maintained in feeder-free condition as this allows to have a pure cell population of human origin. A library of frozen vials of iPSCs can be generated (n=25-50 with 1×10⁶ cells/vial) with passage numbers <10 for each iPSC line derived. After 4-5 additional passages (it will approximately take 15-20 days for this expansion) of a given thawed iPSC vial, a desired number of starting iPSCs for generation of hi-MACS can be reached.

According to further embodiments of the present disclosure, methods of generating patient-specific macrophages, can also comprise: reprogramming one or more somatic cells of a sample from a subject to a pluripotent state; culturing the one or more reprogrammed cells; expanding the one or more reprogrammed cells to a population of induced pluripotent stem cells; and differentiating the one or more reprogrammed cells of the population to a macrophage-like phenotype, wherein the differentiation progresses without embryoid body formation (or embryoid body selection).

In certain aspects of the present disclosure, the reprogramming can comprise delivering, into the one or more somatic or precursor cells, one or more reprogramming expression vectors comprising nucleic acids encoding OCT3/4, SOX2, KFL4, L-MYC, and LIN28. These vectors can be mixed in a ratio to ensure all factors are delivered at approximately equal efficiency as is routinely described in various publications. In certain aspects, reprogramming can also comprise knocking down (or otherwise inhibiting) p53, for example, by the introduction of a vector comprising a shRNA against p53. In some embodiments according to the present disclosure, at least one of the one or more expression vectors can be an episomal expression vector (i.e., one that does not exhibit chromosomal integration or integration into the host genome).

In certain aspects of the present disclosure, iPSCs can be cultured in a culture medium to maintain pluripotency, such as those known in the literature (for example, comprising components such as dulbecco's modified eagle medium (DMEM), fetal bovine serum, L-glutamine, antibiotics (for example, penicillin-streptomycin), β-mercaptoethanol and basic Fibroblast growth factor (B-FGF). In some embodiments, iPSCs can be cultured in media such as mTeSR1.

In certain aspects, the iPSCs can be cultured expanded for about 10 passages to about 25 passages. Karyotyping can be performed to ensure normal karyotype of these iPSCs after expansion/passaging.

In certain aspects, differentiation of iPSCs into macrophages (or macrophage-like cells) comprises removing b-FGF from the culture media and gradually adding IL-3 and M-CSF over a period of time.

In certain aspects, the sample from which cells are to be reprogrammed and differentiated comprises pericranium from a subject. In some embodiments, to derive iPSCs from pericranium, 2 cm×2 cm pericranium can be collected from the skull of patients undergoing brain tumor surgery. The pericranium can be rinsed with Hanks solution, mechanically minced and digested for 2 hours in DMEM/Ham's F12 medium comprising collagenase, human serum and antibiotics. Cells can be pelleted and resuspended in DMEM/Hams F12 medium comprising human serum and plated on dishes to allow for attachment over 1 week. Attached cells containing PDPCs can be collected from the dish and re-programed into iPSCs. In certain aspects, the sample can enriched for periosteum-derived precursor cells (PDPCs) prior to reprogramming.

In certain aspects, following differentiation, one or more therapeutic vectors can be introduced into the differentiated cells with one or more nucleic acids encoding a cytokine (for example, IL-12, IFN-gamma, IL-15, MCP1), growth factor, or other suitable therapeutic protein known in the art to be effective against symptoms of a cancerous malignancy. In certain aspects, the therapeutic vector can comprise therapeutic nucleic acid, each therapeutic nucleic acid independently encoding a cytokine, a checkpoint protein, or a self-destruction protein. In additional embodiments, the one or more therapeutic vectors can encode a gene that expresses a protein that can be used to track the cells after transplantation, for example, eGFP or CD19. In additional embodiments, the one or more therapeutic vectors can be inducible vectors that require the presence of an inducing agent (e.g., tetracycline or doxycycline) to express the gene of interest, such as a cytokine. In some embodiments, the one or more therapeutic vectors can be viral vectors that can be used to package genes of interest into a virus (e.g. a lentivirus) that can be used for transduction of cells as described herein (iPSCs and HI-MACS, for example).

### IV. Pharmaceutical Compositions and Formulations

Compositions comprising one or more reprogrammed and subsequently differentiated cells (i.e., macrophages derived from iPSCs) are described herein. The compositions may further comprise a diluent, solubilizer, emulsifier, preservative, and/or adjuvant to be used with the methods disclosed herein. Such compositions can be used in a subject having or suspected of having a cancerous malignancy that would benefit from any of the compositions described herein. In some embodiments, cells can be resuspended in plasmalyte or isotonic saline with human serum albumin. IA administration typically needs about 20 mls while IV administration needs about 100 mls.

In certain embodiments, acceptable formulation materials preferably are nontoxic to recipients at the dosages and concentrations employed. In certain embodiments, the formulation material(s) are for s.c. and/or I.V. administration (or intra-arterial, IA, formulation). In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Allen (2012) Remington—The Science and Practice of Pharmacy, 22d Edition, Lloyd V, Allen, ed., The Pharmaceutical Press). In certain embodiments, the optimal pharmaceutical composition is determined by one skilled in the art depending upon, for example, the intended route of administration, delivery format and desired dosage. See, for example, Allen (2012) Remington—The Science and Practice of Pharmacy, 22d Edition, Lloyd V, Allen, ed., The Pharmaceutical Press. In certain embodiments, such compositions may influence the physical state, stability, rate of in vivo release and/or rate of in vivo clearance of the cell[s] or compositions as described herein.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a macrophage (or HiMac) prepared by the methods as disclosed herein can be prepared for storage by mixing the selected composition having the desired degree of purity with optional formulation agents (see Allen (2012) Remington—The Science and Practice of Pharmacy, 22d Edition, Lloyd V, Allen, ed., The Pharmaceutical Press) in the form of a lyophilized cake or an aqueous solution. Further, in certain embodiments, a composition comprising a macrophage (or HiMac) prepared by the methods as disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and the like.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments, the pharmaceutical composition can be selected for parenteral delivery. The preparation of such pharmaceutically acceptable compositions is within the ability of one skilled in the art. In certain embodiments when parenteral administration is contemplated, a therapeutic composition can be in the form of a pyrogen-free, parenterally acceptable aqueous solution comprising a macrophage (or HiMac) prepared by the methods as disclosed herein in a pharmaceutically acceptable vehicle. In certain embodiments, a vehicle for parenteral injection is sterile distilled water in which a macrophage (or HiMac) prepared by the methods as disclosed herein is formulated as a sterile, isotonic solution and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

The pharmaceutical composition to be used for in vivo administration typically is sterile. In certain embodiments, sterilization is accomplished by filtration through sterile filtration membranes. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, or emulsion.

In certain embodiments, a pharmaceutical composition can include an effective quantity/amount of a macrophage (or hiMac) (also referred to herein as a “therapeutically effective amount”) prepared by the methods as disclosed herein in a mixture with non-toxic excipients suitable for the manufacture of tablets. Such effective amounts, in some embodiments, can be about 1×10*5, about 1×10*6, or about 1×10*7 cells per kg body weight administered up to 1× for intrarterial delivery OR 1× per week for 3 weeks for intravenous infusion. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

In certain embodiments, the effective amount of a pharmaceutical composition comprising a macrophage (or HiMac) prepared by the methods as disclosed herein to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a cell or population of cells as provided in this disclosure is being used, the route of administration, and the size (body weight, body surface or organ size) and/or condition (the age and general health) of the patient. The clinician can titer the dosage and modify the route of administration to obtain the optimal therapeutic effect. Dosing is discussed further in the next section of this disclosure.

In certain embodiments, the route of administration of the pharmaceutical composition is in accord with known methods, e.g., through injection by intraarterial, intravenous, intraperitoneal, intracerebral (intra-parenchymal), intraventricular, intramuscular, subcutaneously, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired cell has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a macrophage (or HiMac) prepared by the methods as disclosed herein in an ex vivo manner.

### V. Therapeutic Methods

As described herein, the present disclosure provides a method of treating a subject with a cancerous malignancy, comprising administering to the subject an amount or therapeutically effective amount of a macrophage (or HiMac) prepared by the methods as disclosed herein of the present disclosure. In some embodiments, the subject has, is determined to have, or otherwise expected of having a cancerous malignancy, for example, a glioblastoma.

The compositions described herein are useful in, inter alia, methods for treating a cancerous malignancy in a subject. As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have a cancerous malignancy In some embodiments, the subject is diagnosed with a cancerous malignancy. In some embodiments, the subject is a human that is suspected of having a cancerous malignancy. The terms subject and patient are used interchangeably in this disclosure.

The principal symptoms of a cancerous malignancy as those described herein can include headaches, seizures, difficulty speaking and paralysis.

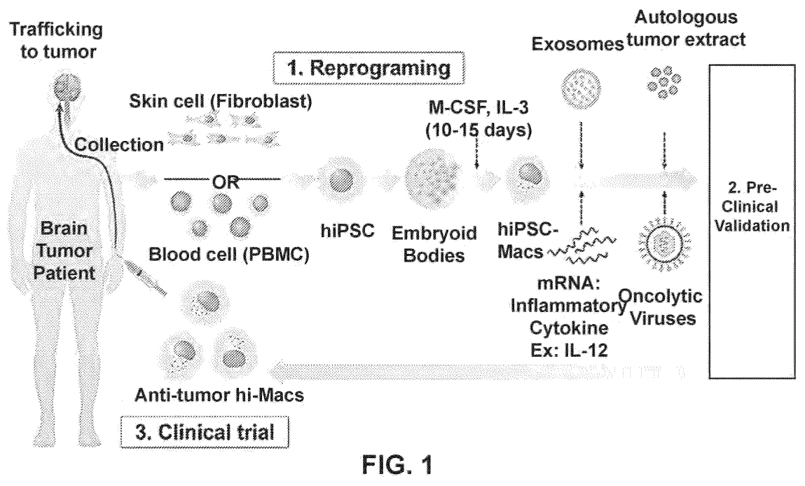

The cells and compositions according to methods of the present disclosure can also be used as a prophylactic therapy for cancerous malignancy disease. The provided antibodies and fragments thereof may be used either in prophylactic and therapeutic administration as well as either by passive immunization with substantially purified polypeptide products and gene therapy by transfer of polynucleotide sequences encoding the product or part thereof. Thus, the provided antibodies and fragments thereof can be administered to high-risk subjects in order to lessen the likelihood and/or severity of cancerous malignancy disease or administered to subjects already evidencing active cancerous malignancy. For example, in some embodiments, compositions and methods as described herein can be used as a tumor antigen to prime the macrophages and then infusing so that they can present antigen to T-cells and then generate anti-tumor t-cell population (see, for example, FIG. 1).

As used herein, administer or administration refers to the act of injecting or otherwise physically delivering a substance as it exists outside the body (e.g., a cell according to methods of the present disclosure or composition comprising such) into a patient, such as by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. Additional information regarding administration is also provided in the prior section of this disclosure.

The compositions can be administered to a subject, e.g., a human subject, using a variety of methods that depend, in part, on the route of administration. The route can be, e.g., intra-arterial injection or infusion (IA), intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing antibodies are well known to those of skill in the art. Generally, such systems should utilize components which will not significantly impair the biological properties of the cells (see, for example, Remington's Pharmaceutical Sciences, 18th edition, 1990, Mack Publishing). Those of skill in the art can readily determine the various parameters and conditions for producing cell based compositions as provided in this disclosure without resort to undue experimentation.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 5,164,188; 4,863,457; and 3,710,795. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems. In some embodiments, a cell or composition of the present disclosure is therapeutically delivered to a subject by way of local administration.

Treating or treatment of any disease or disorder refers to ameliorating a disease or disorder that exists in a subject or a symptom thereof. The term ameliorating refers to any therapeutically beneficial result in the treatment of a disease state, e.g., a cancerous malignancy, lessening in the severity or progression, promoting remission or durations of remission, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying or preventing metastasis. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a cancerous malignancy in a subject by administering a composition as described in this disclosure is considered to be a treatment if there is a 10% reduction in one or more symptoms of the cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used in this disclosure, the term “therapeutically effective amount” refers to an amount of therapeutic agent effective to treat at least one symptom of a disease or disorder in a subject. In other words, such an amount is sufficient to bring about a beneficial or desired clinical effect. As used herein, a “prophylactically effective amount” of a cell or composition as described herein is a dosage large enough to produce the desired effect in the protection of individuals against cancerous malignancy symptoms for a reasonable period of time, such as one to two months or longer following administration. A prophylactically effective amount is not, however, a dosage so large as to cause adverse side effects, such as hyperviscosity syndromes, pulmonary edema, congestive heart failure, and the like. Generally, a prophylactically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. The dosage of the prophylactically effective amount may be adjusted by the individual physician or veterinarian in the event of any complication. In some instances, a prophylactically effective amount may vary from about 0.01 mg/kg to about 50 mg/kg, preferably from about 0.1 mg/kg to about 20 mg/kg, most preferably from about 0.2 mg/kg to about 2 mg/kg, in one or more administrations (priming and boosting).

A pharmaceutical composition can include a therapeutically effective amount or a prophylactically effective amount of a population of cells as described herein. In some embodiments, such effective amounts can be about 1×10*5, about 1×10*6, or about 1×10*7 cells per kg body weight. Additional aspects of such effective amounts can be readily determined by one of ordinary skill in the art as described above. Considerations include the effect of the administered cell[s] or compositions as described herein, or the combinatorial effect of the cell[s] or compositions as described herein with one or more additional active agents, if more than one agent is used in or with the pharmaceutical composition.

Suitable doses for humans of cell[s] or compositions as described herein can further be evaluated in, e.g., Phase I dose escalation studies. See, e.g., van Gurp et al. (2008) Am J Transplantation 8(8): 1711-1718; Hanouska et al. (2007) Clin Cancer Res 13(2, part 1): 523-531; and Hetherington et al. (2006) Antimicrobial Agents and Chemotherapy 50(10): 3499-3500.

Toxicity and therapeutic efficacy of such cell[s] and compositions as described herein can be determined by known pharmaceutical procedures in cell cultures or experimental animals (e.g., animal models of any of the cancers described herein). These procedures can be used, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. A cell[s] or compositions as described herein that exhibits a high therapeutic index is preferred. While formulations that exhibit toxic side effects may be used, care should be taken to design a delivery system that targets such constructs to the site of affected tissue and to minimize potential damage to normal cells and, thereby, reduce side effects.

In some embodiments, a cell[s] or compositions as described herein can be administered to a subject as a monotherapy. Alternatively, the cell[s] or compositions as described herein can be administered in conjunction with other therapies for cancer (combination therapy). In some embodiments and without intending to be limiting, co-therapies that can be administered with aspects of the present disclosure can include radiation therapy prior to systemic HiMacs; and immune check point inhibitors such as nivolumab before and after HiMacs administration. For example, the composition can be administered to a subject at the same time, prior to, or after, a second therapy. In some embodiments, the cell[s] or compositions as described herein and the one or more additional active agents are administered at the same time. Optionally, the cell[s] or compositions as described herein is administered first in time and the one or more additional active agents are administered second in time. In some embodiments, the one or more additional active agents are administered first in time and the cell[s] or compositions as described herein is administered second in time. Optionally, the cell[s] or compositions as described herein and the one or more additional agents are administered simultaneously in the same or different routes. For example, a composition comprising the cell[s] or compositions as described herein optionally contains one or more additional agents.

In some embodiments, described herein are cotherapies with compositions and methods as described herein administered with radiation therapy prior to systemic iMacs or immune check point inhibitors such as nivolumab before and after iMacs administration.

A cell[s] or compositions as described herein can replace or augment a previously or currently administered therapy. For example, upon treating with a cell[s] or composition as described herein, administration of the one or more additional active agents can cease or diminish, e.g., be administered at lower levels or dosages. In some embodiments, administration of the previous therapy can be maintained. In some embodiments, a previous therapy is maintained until the level of the cell[s] or compositions as described herein reaches a level sufficient to provide a therapeutic effect.

Monitoring a subject (e.g., a human patient) for an improvement of cancerous malignancy, as defined herein, means evaluating the subject for a change in a disease parameter, e.g., a reduction in one or more symptoms of cancerous malignancy exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancerous malignancy described herein.

The clinician also selects the frequency of dosing, taking into account the pharmacokinetic parameters of the a macrophage (or HiMac) prepared by the methods as disclosed herein in the formulation used. In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

### EXAMPLES

### Example 1: Generating Human Induced Pluripotent Stem Cell Derived Macrophages (Hi-Macs) for Brain Tumor Therapy

A. Deriving Induced Pluripotent Stem Cells (iPSCs) from Patient's Peripheral Blood

Peripheral blood mononuclear cells (PBMCs) collected from patients were reprogrammed using the standard non-integrating (episomal) expression of human OCT3/4 and shRNA against p53 (pCXLE-hOCT3/4-shp53-F, Addgene cat. #27077), Integration-free (episomal) expression of human SOX2 and KLF4 (pCXLE-hSK, Addgene cat. #27078), and Integration-free (episomal) expression of human L-MYC and LIN28 (pCXLE-UL, Addgene cat. #27080) (Okita, K. et al. A more efficient method to generate integration-free human iPS cells. Nat Methods 8, 409-412 (2011). doi.org: 10.1038/nmeth.1591). Induced pluripotent stem cells were derived in accordance of The Human Embryonic and Induced Pluripotent Stem Cell Oversight Committee (HEIPSCRO) guidelines. The clones were karyotyped at the MDACC's cytogenetic core to determine chromosome status and if they have normal karyotype. iPSC state was verified in vitro (self-renewal assays, karyotyping) and in-vivo (teratoma formation assays) as previously described (Nelakanti, R. V., Kooreman, N. G. & Wu, J. C. Teratoma formation: a tool for monitoring pluripotency in stem cell research. Curr Protoc Stem Cell Biol 32, 4A 8 1-4A 8 17 (2015). doi.org: 10.1002/9780470151808.sc04a08s32, which is incorporated by reference as if fully set forth herein for description of teratoma formation assays).

B. Differentiation of Human iPSCs to Macrophages or Hi-Macs:

According to the present example, iPSCs are cultured in DMEM with 20% knockout serum replacement, 1 mM L-glutamine and 1% penicillin-streptomycin, 0.1 mM β-mercaptoethanol and 10 ng/ml basic Fibroblast growth factor (B-FGF) or mTeSR1 media. Macrophage differentiation was directly induced in the culture dish (without embryoid body selection or formation otherwise) by omitting B-FGF supplementation during passage and adding 10 μM Y-27632 (ROCK inhibitor) to the dish (FIG. 1). After 5 days, X-VIVO 15 (Lonza) with 1 mM L-glutamine, 1% penicillin-streptomycin, 0.05 mM β-mercaptoethanol, 25 ng/ml IL-3 and 50 ng/ml M-CSF was added, as described in section 0118. Supernatant/conditioned media with hi-MACS are collected and can then be terminally differentiated by culturing in media with 50 ng/ml of M-CSF for 7 days (data not shown; see immunocytochemistry shown in FIG. 1B of U.S. Provisional Patent Appl. No. 63/495,953). After 7 days Hi-Macs were selectively detached via 10 minutes exposure to PBS without Ca⁺⁺Mg⁺. The collected Hi-Macs were pelleted and sent for flow cytometry analysis. Cells can be pelleted by centrifuging at 300 g for 10 minutes at room temperature in a clinical centrifuge. The remaining attached cells were maintained in culture with X-VIVO 15 (Lonza) with 1 mM L-glutamine, 1% penicillin-streptomycin, 0.05 mM β-mercaptoethanol, 25 ng/ml IL-3 and 50 ng/ml M-CSF for next round of Hi-Macs harvesting.

### C. Flow Cytometry Characterization of Hi-Macs

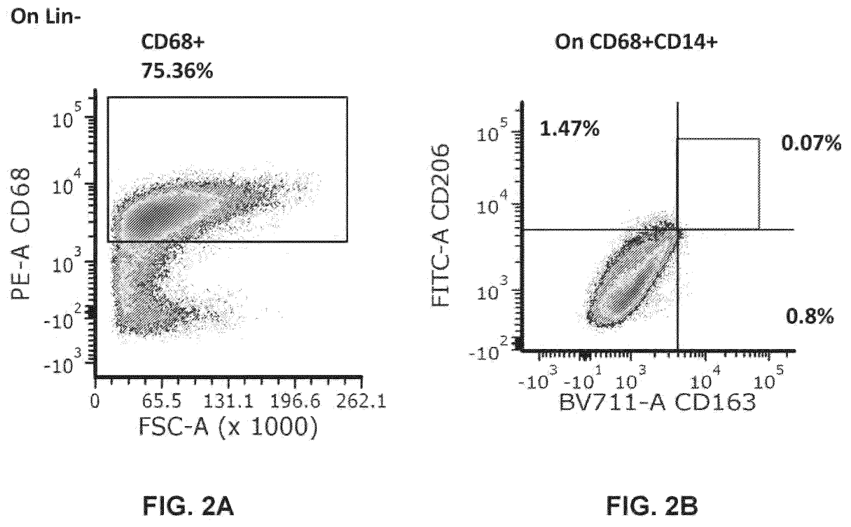

The immune-phenotype of Hi-Macs was characterized by multi-color flow cytometry (Fortessa X-20). Live/Dead Fixable Aqua staining was first performed to allow the discrimination of Live/Dead cells. CD14+CD68+ macrophages were identified by a negative exclusion gating strategy previously described by the research group of the present inventors (Banerjee, P. et al. Trabectedin Reveals a Strategy of Immunomodulation in Chronic Lymphocytic Leukemia. Cancer Immunol Res 7, 2036-2051 (2019). doi.org: 10.1158/2326-6066.CIR-19-0152, incorporated by reference as if fully set forth herein regarding the negative exclusion gating strategy). CD66b-neutrophils were excluded, then, using a lineage (Lin) cocktail including mAbs to CD3, CD19, CD20, and CD56, T cells, B cells, and NK cells were excluded, respectively (>60% Lin−). Further characterization revealed that up to 40% of Lin-cells expressed CD68+, a pan macrophage/phagocyte marker (FIG. 2A). Over 90% of the CD68+ macrophages were predominantly M1 pro-inflammatory/anti-tumor macrophages as they lacked the classic pro-tumor M2 macrophage markers CD206−CD163− (FIG. 2B). Fluorescence immunocytochemistry also showed that hi-Macs derived from iPSC Line 1 (male) and Line 2 (female) express classic macrophage markers IBA-1 and CD68, with the majority of the cells were found to be positive for both markers (data not shown; see FIG. 1B of U.S. Provisional Patent Appl. No. 63/495,953).

### Example 2: IPSC Derived Macrophage Cell-Based Therapy for Cancer and Inflammatory Conditions

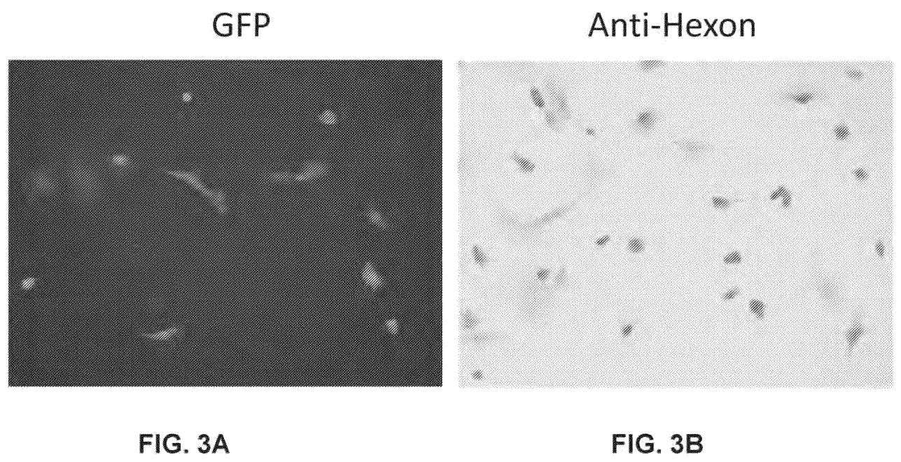

Operation: To obtain patient specific source for iPSCs, skin cells or blood or pericranium can be collected from patients. Cells from these samples can be reprogrammed into iPSCs using published protocol. The iPSCs can then be differentiated into macrophages using published protocols (MDA-HiMacs). MDA-HiMacs can be collected from the medium once a week over 3 weeks for up to 30×10e6 cells per week. Next, patient-derived iMacs can be transduced with lentivirus bearing cDNA encoding non-functional truncated human CD19t (to detect exogenous HiMacs), herpes simplex virus thymidine kinase (HSV/TK; suicide gene to eliminate HiMacs, e.g., SEQ ID NO:5), or a diptheria toxis-associated suicide gene); biologically active human IL-12 (hIL-12) sub-units (hp35 and hp40 cDNAs, corresponding to IL-12a and IL-12ß subunits, respectively) and WPRE (Woodchuck Hepatitis Virus Post-Transcriptional Regulatory Element). As shown in FIGS. 3A-3B, these cells are amenable to viral transduction. Examples of vectors that can be used with these elements include pCMV-VSV-G envelope and adenovirus type 5 vectors. WPRE can be used to determine integrated lentiviral copy number via real-time PCR. Viral transduction can be titrated to yield<5 integrated lentiviral copies per genome (in accordance with FDA guidelines). Biologically active IL-12 secretion can be verified by ELISA and by incubation with autologous CD3+ T-cells in vitro to assess for T-cell activation based on IFN-γ release. Sensitivity of TK-expressing HiMacs to ganciclovir (GCV; 1-10 μM) will also be verified in vitro. MDA-HiMacs will also be transduced with Delta-24RGD oncolytic virus. The virus bearing MDA-HiMacs will be delivered to tumors in the patient from which the iPSC was derived directly or via arterial access.

Characteristics: 2 MDA-HiMac cell lines have previously been generated from human induced pluripotent stem cells using macrophage colony stimulating factor (M-CSF) and interleukin 3 (IL-3) (as shown in the Figures). The immune-phenotype of both MDA-HiMacs (Line 1 male and Line 2 female) by multi-color flow cytometry (Fortessa X-20). Live/Dead Fixable Aqua staining was first performed to allow the discrimination of Live/Dead cells. CD14+CD68+ macrophages were identified by a negative exclusion gating strategy previously described. CD66b-neutrophils were excluded, then, using a lineage (Lin) cocktail including monoclonal antibodies to CD3, CD19, CD20, and CD56, T cells, B cells, and NK cells were excluded, respectively (>60% Lin−; unpublished data). Further characterization revealed that up to 40% of Lin-cells expressed CD68+, a pan macrophage/phagocyte marker and Iba1. Over 90% of the CD68+ macrophages were predominantly pro-inflammatory/anti-tumor macrophages as they lacked the classic pro-tumor macrophage markers CD206-CD163. Based on cell numbers, this protocol in a 6-well plate generated on average 4×10⁶ HiMacs. This indicates that the protocol could be scaled up to bioreactors that would allow for harvest of up to 30×10e6 HiMacs for cell-based therapy in patients.

### Example 3: Harvest and Culture of Pericranium Progenitor Cells Derived from Human Pericranium

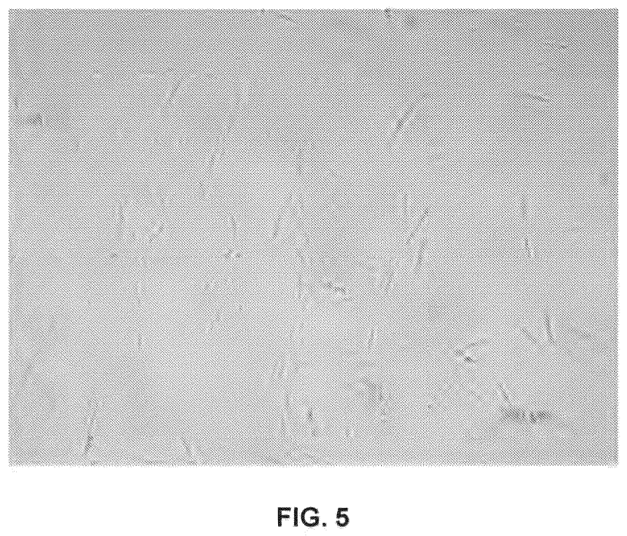

In brief, a 1×1 inch of human pericranium was harvested during the exposure for brain surgery. The tissue was taken to the lab and dissociated mechanically and enzymatically with Trypsin. The dissociated tissue was cultured in DMEM)/Ham's F12 medium containing 10,000 U/ml collagenase II, 10% human allogenic serum 2.5% Hepes and 1% penicillin/streptomycin solution. The progenitor cells were identified and expanded from the monolayer growing at the bottom of the dish (FIG. 5). Progenitor cells were identified by adherent and monolayer morphology with proliferative abilities.

### Example 4: IMACS Derived from Primary Human Pericranium Progenitor Cells

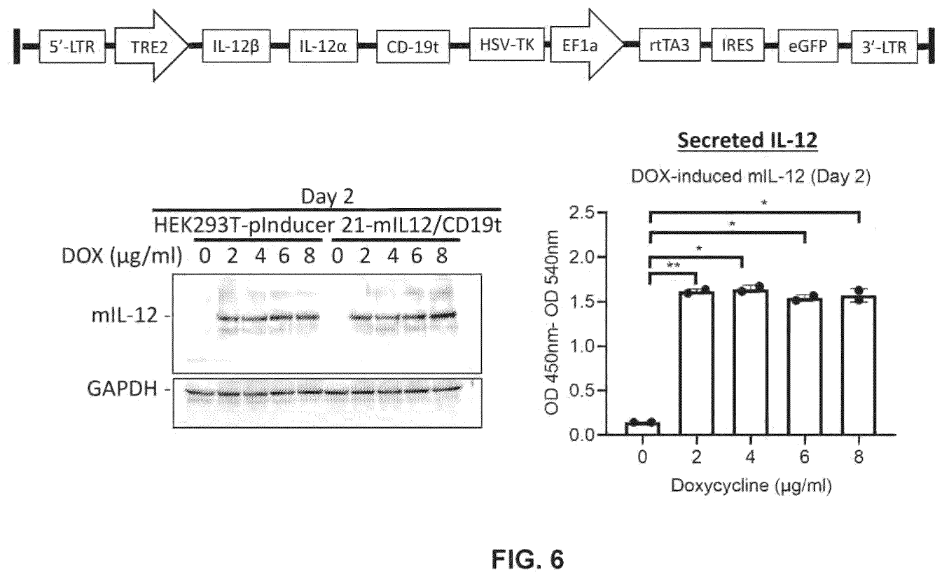

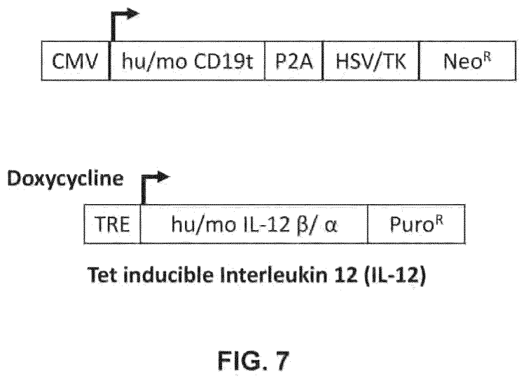

Progenitor cells can be isolated, cultured, and expanded from the pericranium tissue of GBM patients undergoing brain surgery (FIG. 5 and Example 3 above). These progenitor cells can be reprogrammed into induced pluripotent stem cells (iPSCs; by methods as described at least in Example 1 above, for example). iPSCs can then be differentiated into macrophages (iMacs). iMacs can be transduced with a virus (for example, a lentivirus or Delta-24RGD oncolytic virus) bearing cDNA encoding: non-functional truncated CD19t (to detect exogenous iMacs; for example NCBI GenBank ID: AAL57719.1; SEQ ID NO:3; or human CD19t (SEQ ID NO: 14)); herpes simplex virus thymidine kinase (HSV/TK; a suicide gene to eliminate iMacs); biologically active human IL-12 (hIL-12) sub-units (hp35 and hp40 cDNAs, for example, NCBI GenBank Accession: AAD56385.1 and NCBI GenBank Accession: AAD56386.1, respectively; for example SEQ ID NOs: 16 and 17); and WPRE (Woodchuck Hepatitis Virus Post-Transcriptional Regulatory Element). The top panel of FIG. 6 shows a schematic of a 1^{ST} Generation of a construct and FIG. 7 shows a schematic of a 2^{ND} generation of an IL-12 construct where only IL-12 expression is Dox inducible, while CD19t and HSV/TK are constitutive. Biologically active IL-12 secretion can be verified by conventional measures such as ELISA and western blot, for example, as shown in bottom left and bottom right panels of FIG. 6 for a 1st Generation IL-12 construct. Additionally, cells can be incubated with autologous CD3+ T-cells in vitro to assess for T-cell activation based on IFN-γ release. Sensitivity of TK-expressing iMacs to ganciclovir (GCV; 1-10 μM) can also be verified in vitro.

### Materials and Methods for Example 4

For the in vitro assays above, in brief, peripherally derived CD3+ sorted T-cells were activated and expanded in-vitro using Dynabeads (CD3/CD28 co-stimulation) for 48 hours. Following activation, the CD3+ T-cells were expanded over several days. The Dynabeads were released from the T-cells and the activated/expanded T-cells were ready for analysis. To test whether IL-12 secreted from the construct would induce IFN-γ release from the T-cells, the supernatant from doxycycline treated cells with the IL-12 construct was added to the T-cells in a 96-well plate/triplicates per condition (positive control recombinant IL-12 was added to T-cells in a separate well). Supernatant was harvested from the T-cells at multiple type points following incubation. IFN-γ in the supernatant was measured using an ELISA. For the TK assay, cells stably expressing IL-12-HSV TK were incubated with 0, 1, 5 and 10 μM of Gancyclovir for 24 hrs in a 96 well plate/triplicates. Cell survival and susceptibility was determined by the percentage of cells remaining in the HSV/TK expressing wells relative to untreated wells (0 uM).

### Example 5: Evaluation of IMAC Efficacy In Vivo

To recapitulate a human systemic immune compartment in vivo, NSG-SGM3 immunodeficient mice (NOD.Cg-Prkdc^{scid} Il2rg^{tm1Wjl} Tg (CMV-IL3,CSF2,KITLG)1Eav/MloySzJ) can be used, for example. Unlike traditional NSG mice, NSG-SGM3 mice express human IL-3, CSF2, KITLG which enhance myeloid cell engraftment. Following human CD34⁺ HSC transplant, NSG-SGM3 mice (relative to NSG mice) demonstrate higher engraftment and persistence of dendritic cells, macrophages, B-cells and CD4/CD8/FoxP3 regulatory T-cells. After sub-lethal whole-body radiation (4Gy), an amount of (for example, 5×10⁴) patient-derived human CD34⁺ HSCs/mouse can be injected via tail vein into NSG-SGM3 mice and their engraftment can be verified by characterizing immune profiles (T-cells, B-cells, dendritic cells and monocytes/macrophages) by flow cytometry at time points after injection (6- and 10-weeks post-injection, for example). Following verification of engraftment, luciferase-expressing human glioblastoma stem cells (GSCs) can be stereotactically implanted (1×10⁵ cells, for example) from each patient into the cortex of NSG-SGM3 mice (n=20 mice per GSC line) at a time point post HSC transplantation (10 weeks, for example). Autologous iMacs bearing constitutive CD19/HSV TK construct and the Dox-inducible IL-12 construct (FIG. 7) can be administered to each mouse (for example, 16 weeks post HSC transplantation and 6 weeks post GSC transplantation; 2×10⁵ cells, for example). In the present example, the iMacs are autologous because the iMacs are human derived and the mice are humanized mice with an immune system from the same human the iMacs were derived.

Tumor growth can then be monitored via weekly bioluminescence imaging (BLI), for example, at various time points. Changes in circulating immune cell repertoire can also be monitored using flow cytometry for standard immune populations. To determine the extent to which the IL-12 iMacs traffic to, persist in, retain anti-tumor polarization and recruit activated T cells, animals can be serially euthanized for immunohistochemistry (for example, 2 animals per week), single cell RNA sequencing (scRNA seq,) and collection of peripheral organs (lung, liver and spleen) for bioavailability studies. Ganciclovir (GCV) can also be administered to animals (for example, to 3 mice per group at 100 mg/kg GCV, the skilled artisan would be able to determine doses above and below this range) on a post-treatment day (for example, post-treatment day 3) and their brains can be harvested after a period of time (for example, after 5 days) to verify iMac elimination based on anti-CD19 immunohistochemistry (relative to mice not receiving GCV).

### REFERENCES

1 Stupp, R. et al. Radiotherapy plus concomitant and adjuvant temozolomide for glioblastoma. N Engl J Med 352, 987-996 (2005). doi.org: 10.1056/NEJMoa043330
2 Tawbi, H. A. et al. Relatlimab and Nivolumab versus Nivolumab in Untreated Advanced Melanoma. N Engl J Med 386, 24-34 (2022). doi.org: 10.1056/NEJMoa2109970
3 Sheykhhasan, M., Manoochehri, H. & Dama, P. Use of CAR T-cell for acute lymphoblastic leukemia (ALL) treatment: a review study. Cancer Gene Ther 29, 1080-1096 (2022). doi.org: 10.1038/s41417-021-00418-1
4 Brempelis, K. J. et al. Genetically engineered macrophages persist in solid tumors and locally deliver therapeutic proteins to activate immune responses. J Immunother Cancer 8 (2020). doi.org: 10.1136/jitc-2020-001356
5 Klichinsky, M. et al. Human chimeric antigen receptor macrophages for cancer immunotherapy. Nat Biotechnol 38, 947-953 (2020). doi.org: 10.1038/s41587-020-0462-y
6 Okita, K. et al. A more efficient method to generate integration-free human iPS cells. Nat Methods 8, 409-412 (2011). doi.org: 10.1038/nmeth.1591
7 Nelakanti, R. V., Kooreman, N. G. & Wu, J. C. Teratoma formation: a tool for monitoring pluripotency in stem cell research. Curr Protoc Stem Cell Biol 32, 4A 8 1-4A 8 17 (2015). doi.org: 10.1002/9780470151808.sc04a08s32
8 Banerjee, P. et al. Trabectedin Reveals a Strategy of Immunomodulation in Chronic Lymphocytic Leukemia. Cancer Immunol Res 7, 2036-2051 (2019). doi.org: 10.1158/2326-6066.CIR-19-0152

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

INFORMAL TABLE OF SEQUENCES

| SEQ ID | | |
|---|---|---|
| NO: | SEQUENCE | DESCRIPTION |
| 1 | ACGCAGAAGCCGGGTGCCCCAGGGAGCCTGGCTACC | Pre-CDS Sequence |
| 2 | ATGCCATCTCCTCTCCCTGTCTCCTTCCTCCTCTTTCTTAC | Signal Peptide |
| | CTTAGTAGGAGGC | |
| 3 | AGGCCCCAGAAGTCCTTACTGGTGGAGGTAGAAGAGGGA | CD-19t |
| | GGCAATGTTGTGCTGCCATGCCTCCCGGACTCCTCACCTG | |
| | TCTCTTCTGAGAAGCTGGCTTGGTATCGAGGTAACCAGTC | |
| | AACACCCTTCCTGGAGCTGAGCCCCGGGTCCCCTGGCCTG | |
| | GGATTGCACGTGGGGTCCCTGGGCATCTTGCTAGTGATTG | |
| | TCAATGTCTCAGACCATATGGGGGGCTTCTACCTGTGCCA | |
| | GAAGAGGCCCCCTTTCAAGGACATCTGGCAGCCTGCCTG | |
| | GACAGTGAACGTGGAGGATAGTGGGGAGATGTTCCGGTG | |
| | GAATGCTTCAGACGTCAGGGACCTGGACTGTGACCTAAG | |
| | GAACAGGTCCTCTGGGAGCCACAGGTCCACTTCTGGTTCC | |
| | CAGCTGTATGTGTGGGCTAAAGACCATCCTAAGGTCTGG | |
| | GGAACAAAGCCTGTATGTGCCCCTCGGGGGAGCAGTTTG | |
| | AATCAGAGTCTAATCAACCAAGATCTCACTGTGGCACCC | |
| | GGCTCCACACTTTGGCTGTCCTGTGGGGTACCCCCTGTCC | |
| | CAGTGGCCAAAGGCTCCATCTCCTGGACCCATGTGCATCC | |
| | TAGGAGACCTAATGTTTCACTACTGAGCCTAAGCCTTGGG | |
| | GGAGAGCACCCGGTCAGAGAGATGTGGGTTTGGGGGTCT | |
| | CTTCTGCTTCTGCCCCAAGCCACAGCTTTAGATGAAGGCA | |
| | CCTATTATTGTCTCCGAGGAAACCTGACCATCGAGAGGC | |
| | ACGTGAAGGTCATTGCAAGGTCAGTGTGGCTCTGGCTGTT | |
| | GAGAACTGGTGGATGGATAGTCCCAGTGGTGACTTTAGT | |
| | ATATGTCATCTTC | |
| 4 | GCCACCAACTTCTCCCTGCTGAAGCAGGCCGGCGACGTG | P2A |
| | GAGGAGAACCCCGGCCCC | |
| 5 | GCTTCGTACCCCGGCCATCAACACGCGTCTGCGTTCGACC | HSV-TK |
| | AGGCTGCGCGTTCTCGCGGCCATAGCAACCGACGTACGG | |
| | CGTTGCGCCCTCGCCGGCAGCAAGAAGCCACGGAAGTCC | |
| | GCCCGGAGCAGAAAATGCCCACGCTACTGCGGGTTTATA | |
| | TAGACGGTCCCCACGGGATGGGGAAAACCACCACCACGC | |
| | AACTGCTGGTGGCCCTGGGTTCGCGCGACGATATCGTCTA | |
| | CGTACCCGAGCCGATGACTTACTGGCGGGTGCTGGGGGC | |
| | TTCCGAGACAATCGCGAACATCTACACCACACAACACCG | |
| | CCTCGACCAGGGTGAGATATCGGCCGGGGACGCGGCGGT | |
| | GGTAATGACAAGCGCCCAGATAACAATGGGCATGCCTTA | |
| | TGCCGTGACCGACGCCGTTCTGGCTCCTCATATCGGGGGG | |
| | GAGGCTGGGAGCTCACATGCCCCGCCCCCGGCCCTCACC | |
| | CTCATCTTCGACCGCCATCCCATCGCCGCCCTCCTGTGCT | |
| | ACCCGGCCGCGCGGTACCTTATGGGCAGCATGACCCCCC | |
| | AGGCCGTGCTGGCGTTCGTGGCCCTCATCCCGCCGACCTT | |
| | GCCCGGCACCAACATCGTGCTTGGGGCCCTTCCGGAGGA | |
| | CAGACACATCGACCGCCTGGCCAAACGCCAGCGCCCCGG | |
| | CGAGCGGCTGGACCTGGCTATGCTGGCTGCGATTCGCCG | |
| | CGTTTACGGGCTACTTGCCAATACGGTGCGGTATCTGCAG | |
| | TGCGGCGGGTCGTGGCGGGAGGACTGGGGACAGCTTTCG | |
| | GGGACGGCCGTGCCGCCCCAGGGTGCCGAGCCCCAGAGC | |
| | AACGCGGGCCCACGACCCCATATCGGGGACACGTTATTT | |
| | ACCCTGTTTCGGGCCCCCGAGTTGCTGGCCCCCAACGGCG | |
| | ACCTGTATAACGTGTTTGCCTGGGCCTTGGACGTCTTGGC | |
| | CAAACGCCTCCGTTCCATGCACGTCTTTATCCTGGATTAC | |
| | GACCAATCGCCCGCCGGCTGCCGGGACGCCCTGCTGCAA | |
| | CTTACCTCCGGGATGGTCCAGACCCACGTCACCACCCCCG | |
| | GCTCCATACCGACGATATGCGACCTGGCGCGCACGTTTGC | |
| | CCGGGAGATGGGGGAGGCTAACT | |
| 6 | gaattttaacaaaatattaacgcttacaatttaggtggcacttttcggggaaatgtgcgcggaacc | pLenti CMV GFP |
| | cctatttgtttatttttctaaatacattcaaatatgtatccgctcatgagacaataaccctgataaatgc | Neo (Plasmid # |
| | ttcaataatattgaaaaaggaagagtatgagtattcaacatttccgtgtcgcccttattcccttttttg | 17447, from |
| | cggcattttgccttcctgtttttgctcacccagaaacgctggtgaaagtaaaagatgctgaagatc | addgene.org) |
| | agttgggtgcacgagtgggttacatcgaactggatctcaacagcggtaagatccttgagagtttt | |
| | cgccccgaagaacgttttccaatgatgagcacttttaaagttctgctatgtggcgcggtattatcc | |
| | cgtattgacgccgggcaagagcaactcggtcgccgcatacactattctcagaatgacttggttg | |
| | agtactcaccagtcacagaaaagcatcttacggatggcatgacagtaagagaattatgcagtgc | |
| | tgccataaccatgagtgataacactgcggccaacttacttctgacaacgatcggaggaccgaa | |
| | ggagctaaccgcttttttgcacaacatgggggatcatgtaactcgccttgatcgttgggaaccgg | |
| | agctgaatgaagccataccaaacgacgagcgtgacaccacgatgcctgtagcaatggcaaca | |
| | acgttgcgcaaactattaactggcgaactacttactctagcttcccggcaacaattaatagactgg | |
| | atggaggcggataaagttgcaggaccacttctgcgctcggcccttccggctggctggtttattgc | |
| | tgataaatctggagccggtgagcgtgggtctcgcggtatcattgcagcactggggccagatgg | |
| | taagccctcccgtatcgtagttatctacacgacggggagtcaggcaactatggatgaacgaaat | |
| | agacagatcgctgagataggtgcctcactgattaagcattggtaactgtcagaccaagtttactc | |
| | atatatactttagattgatttaaaacttcatttttaatttaaaaggatctaggtgaagatcctttttgata | |
| | atctcatgaccaaaatcccttaacgtgagttttcgttccactgagcgtcagaccccgtagaaaag | |
| | atcaaaggatcttcttgagatcctttttttctgcgcgtaatctgctgcttgcaaacaaaaaaaccac | |
| | cgctaccagcggtggtttgtttgccggatcaagagctaccaactctttttccgaaggtaactggct | |
| | tcagcagagcgcagataccaaatactgttcttctagtgtagccgtagttaggccaccacttcaag | |
| | aactctgtagcaccgcctacatacctcgctctgctaatcctgttaccagtggctgctgccagtgg | |
| | cgataagtcgtgtcttaccgggttggactcaagacgatagttaccggataaggcgcagcggtc | |
| | gggctgaacggggggttcgtgcacacagcccagcttggagcgaacgacctacaccgaactg | |
| | agatacctacagcgtgagctatgagaaagcgccacgcttcccgaagggagaaaggcggaca | |
| | ggtatccggtaagcggcagggtcggaacaggagagcgcacgagggagcttccagggggaa | |
| | acgcctggtatctttatagtcctgtcgggtttcgccacctctgacttgagcgtcgatttttgtgatgc | |
| | tcgtcaggggggcggagcctatggaaaaacgccagcaacgcggcctttttacggttcctggcc | |
| | ttttgctggccttttgctcacatgttctttcctgcgttatcccctgattctgtggataaccgtattaccg | |
| | cctttgagtgagctgataccgctcgccgcagccgaacgaccgagcgcagcgagtcagtgagc | |
| | gaggaagcggaagagcgcccaatacgcaaaccgcctctccccgcgcgttggccgattcatta | |
| | atgcagctggcacgacaggtttcccgactggaaagcgggcagtgagcgcaacgcaattaatg | |
| | tgagttagctcactcattaggcaccccaggctttacactttatgcttccggctcgtatgttgtgtgg | |
| | aattgtgagcggataacaatttcacacaggaaacagctatgaccatgattacgccaagcgcgca | |
| | attaaccctcactaaagggaacaaaagctggagctgcaagcttaatgtagtcttatgcaatactct | |
| | tgtagtcttgcaacatggtaacgatgagttagcaacatgccttacaaggagagaaaaagcaccg | |
| | tgcatgccgattggtggaagtaaggtggtacgatcgtgccttattaggaaggcaacagacgggt | |
| | ctgacatggattggacgaaccactgaattgccgcattgcagagatattgtatttaagtgcctagct | |
| | cgatacataaacgggtctctctggttagaccagatctgagcctgggagctctctggctaactagg | |
| | gaacccactgcttaagcctcaataaagcttgccttgagtgcttcaagtagtgtgtgcccgtctgtt | |
| | gtgtgactctggtaactagagatccctcagacccttttagtcagtgtggaaaatctctagcagtgg | |
| | cgcccgaacagggacttgaaagcgaaagggaaaccagaggagctctctcgacgcaggactc | |
| | ggcttgctgaagcgcgcacggcaagaggcgaggggcggcgactggtgagtacgccaaaaa | |
| | ttttgactagcggaggctagaaggagagagatgggtgcgagagcgtcagtattaagcggggg | |
| | agaattagatcgcgatgggaaaaaattcggttaaggccagggggaaagaaaaaatataaatta | |
| | aaacatatagtatgggcaagcagggagctagaacgattcgcagttaatcctggcctgttagaaa | |
| | catcagaaggctgtagacaaatactgggacagctacaaccatcccttcagacaggatcagaag | |
| | aacttagatcattatataatacagtagcaaccctctattgtgtgcatcaaaggatagagataaaag | |
| | acaccaaggaagctttagacaagatagaggaagagcaaaacaaaagtaagaccaccgcaca | |
| | gcaagcggccgctgatcttcagacctggaggaggagatatgagggacaattggagaagtgaa | |
| | ttatataaatataaagtagtaaaaattgaaccattaggagtagcacccaccaaggcaaagagaa | |
| | gagtggtgcagagagaaaaaagagcagtgggaataggagctttgttccttgggttcttgggag | |
| | cagcaggaagcactatgggcgcagcgtcaatgacgctgacggtacaggccagacaattattgt | |
| | ctggtatagtgcagcagcagaacaatttgctgagggctattgaggcgcaacagcatctgttgca | |
| | actcacagtctggggcatcaagcagctccaggcaagaatcctggctgtggaaagatacctaaa | |
| | ggatcaacagctcctggggatttggggttgctctggaaaactcatttgcaccactgctgtgccttg | |
| | gaatgctagttggagtaataaatctctggaacagatttggaatcacacgacctggatggagtgg | |
| | gacagagaaattaacaattacacaagcttaatacactccttaattgaagaatcgcaaaaccagca | |
| | agaaaagaatgaacaagaattattggaattagataaatgggcaagtttgtggaattggtttaacat | |
| | aacaaattggctgtggtatataaaattattcataatgatagtaggaggcttggtaggtttaagaata | |
| | gtttttgctgtactttctatagtgaatagagttaggcagggatattcaccattatcgtttcagaccca | |
| | cctoccaaccccgaggggacccgacaggcccgaaggaatagaagaagaaggtggagaga | |
| | gagacagagacagatccattcgattagtgaacggatctcgacggtatcggttaacttttaaaaga | |
| | aaaggggggattggggggtacagtgcaggggaaagaatagtagacataatagcaacagaca | |
| | tacaaactaaagaattacaaaaacaaattacaaaattcaaaattttategataagcttgggagttcc | |
| | gcgttacataacttacggtaaatggcccgcctggctgaccgcccaacgacccccgcccattga | |
| | cgtcaataatgacgtatgttcccatagtaacgccaatagggactttccattgacgtcaatgggtgg | |
| | agtatttacggtaaactgcccacttggcagtacatcaagtgtatcatatgccaagtacgcccccta | |
| | ttgacgtcaatgacggtaaatggcccgcctggcattatgcccagtacatgaccttatgggactttc | |
| | ctacttggcagtacatctacgtattagtcatcgctattaccatggtgatgcggttttggcagtacatc | |
| | aatgggcgtggatagcggtttgactcacggggatttccaagtctccaccccattgacgtcaatg | |
| | ggagtttgttttggcaccaaaatcaacgggactttccaaaatgtcgtaacaactccgccccattga | |
| | cgcaaatgggcggtaggcgtgtacggtgggaggtctatataagcagagctcgtttagtgaacc | |
| | gtcagatcgcctggagacgccatccacgctgttttgacctccatagaagacaccgactctagag | |
| | gatccaccggtcgccaccatggtgagcaagggcgaggagctgttcaccggggtggtgcccat | |
| | cctggtcgagctggacggcgacgtaaacggccacaagttcagcgtgtccggcgagggcgag | |
| | ggcgatgccacctacggcaagctgaccctgaagttcatctgcaccaccggcaagctgcccgt | |
| | gccctggcccaccctcgtgaccaccctgacctacggcgtgcagtgcttcagccgctaccccga | |
| | ccacatgaagcagcacgacttcttcaagtccgccatgcccgaaggctacgtccaggagcgca | |
| | ccatcttcttcaaggacgacggcaactacaagacccgcgccgaggtgaagttcgagggcgac | |
| | accctggtgaaccgcatcgagctgaagggcatcgacttcaaggaggacggcaacatcctggg | |
| | gcacaagctggagtacaactacaacagccacaacgtctatatcatggccgacaagcagaaga | |
| | acggcatcaaggtgaacttcaagatccgccacaacatcgaggacggcagcgtgcagctcgcc | |
| | gaccactaccagcagaacacccccatcggcgacggccccgtgctgctgcccgacaaccact | |
| | acctgagcacccagtccgccctgagcaaagaccccaacgagaagcgcgatcacatggtcctg | |
| | ctggagttcgtgaccgccgccgggatcactctcggcatggacgagctgtacaagtaaagcgg | |
| | ccgcgtcgacaatcaacctctggattacaaaatttgtgaaagattgactggtattcttaactatgtt | |
| | gctccttttacgctatgtggatacgctgctttaatgcctttgtatcatgctattgcttcccgtatggctt | |
| | tcattttctcctccttgtataaatcctggttgctgtctctttatgaggagttgtggcccgttgtcaggc | |
| | aacgtggcgtggtgtgcactgtgtttgctgacgcaacccccactggttggggcattgccaccac | |
| | ctgtcagctcctttccgggactttcgctttccccctccctattgccacggcggaactcategccgc | |
| | ctgccttgcccgctgctggacaggggctcggctgttgggcactgacaattccgtggtgttgtcg | |
| | gggaagctgacgtcctttccatggctgctcgcctgtgttgccacctggattctgcgcgggacgtc | |
| | cttctgctacgtcccttcggccctcaatccagcggaccttccttcccgcggcctgctgccggctc | |
| | tgcggcctcttccgcgtcttcgccttcgccctcagacgagtcggatctccctttgggccgcctcc | |
| | ccgcctggaattctgcagatatccggactagtgatctaattctaccgggtaggggaggcgctttt | |
| | cccaaggcagtctggagcatgcgctttagcagccccgctgggcacttggcgctacacaagtg | |
| | gcctctggcctcgcacacattccacatccaccggtaggcgccaaccggctccgttctttggtgg | |
| | ccccttcgcgccaccttctactcctcccctagtcaggaagttcccccccgccccgcagctegcg | |
| | tcgtgcaggacgtgacaaatggaagtagcacgtctcactagtctcgtgcagatggacagcacc | |
| | gctgagcaatggaagcgggtaggcctttggggcagcggccaatagcagctttgctccttcgctt | |
| | tctgggctcagaggctgggaaggggtgggtccgggggcgggctcaggggcgggctcaggg | |
| | gcgggggggcgcccgaaggtcctccggaggcccggcattctgcacgcttcaaaagcgcac | |
| | gtctgccgcgctgttctcctcttcctcatctccgggcctttcgacctgcagccaatatgggatcgg | |
| | ccattgaacaagatggattgcacgcaggttctccggccgcttgggtggagaggctattcggcta | |
| | tgactgggcacaacagacaatcggctgctctgatgccgccgtgttccggctgtcagcgcaggg | |
| | gcgcccggttctttttgtcaagaccgacctgtccggtgccctgaatgaactgcaggacgaggca | |
| | gcgcggctatcgtggctggccacgacgggcgttccttgcgcagctgtgctcgacgttgtcactg | |
| | aagcgggaagggactggctgctattgggcgaagtgccggggcaggatctcctgtcatctcac | |
| | cttgctcctgccgagaaagtatccatcatggctgatgcaatgcggcggctgcatacgcttgatcc | |
| | ggctacctgcccattcgaccaccaagcgaaacatcgcatcgagcgagcacgtactcggatgg | |
| | aagccggtcttgtcgatcaggatgatctggacgaagagcatcaggggctcgcgccagccgaa | |
| | ctgttcgccaggctcaaggcgcgcatgcccgacggcgaggatctcgtcgtgacccatggcga | |
| | tgcctgcttgccgaatatcatggtggaaaatggccgcttttctggattcatcgactgtggccggct | |
| | gggtgtggcggaccgctatcaggacatagcgttggctacccgtgatattgctgaagagcttggc | |
| | ggcgaatgggctgaccgcttcctcgtgctttacggtatcgccgctcccgattcgcagcgcatcg | |
| | ccttctatcgccttcttgacgagttcttctgaggggatccgtcgagggcccggtacctttaagacc | |
| | aatgacttacaaggcagctgtagatcttagccactttttaaaagaaaaggggggactggaaggg | |
| | ctagctcactcccaacgaagacaagatctgctttttgcttgtactgggtctctctggttagaccaga | |
| | tctgagcctgggagctctctggctaactagggaacccactgcttaagcctcaataaagcttgcct | |
| | tgagtgcttcaagtagtgtgtgcccgtctgttgtgtgactctggtaactagagatccctcagaccc | |
| | ttttagtcagtgtggaaaatctctagcagtagtagttcatgtcatcttattattcagtatttataacttgc | |
| | aaagaaatgaatatcagagagtgagaggaacttgtttattgcagcttataatggttacaaataaag | |
| | caatagcatcacaaatttcacaaataaagcatttttttcactgcattctagttgtggtttgtccaaact | |
| | catcaatgtatcttatcatgtctggctctagctatcccgcccctaactccgcccatcccgcccctaa | |
| | ctccgcccagttccgcccattctccgccccatggctgactaattttttttatttatgcagaggccga | |
| | ggccgcctcggcctctgagctattccagaagtagtgaggaggcttttttggaggcctagggacg | |
| | tacccaattcgccctatagtgagtcgtattacgcgcgctcactggccgtcgttttacaacgtcgtg | |
| | actgggaaaaccctggcgttacccaacttaatcgccttgcagcacatccccctttcgccagctg | |
| | gcgtaatagcgaagaggcccgcaccgatcgcccttcccaacagttgcgcagcctgaatggcg | |
| | aatgggacgcgccctgtagcggcgcattaagcgcggcgggtgtggtggttacgcgcagcgtg | |
| | accgctacacttgccagcgccctagcgcccgctcctttcgctttcttcccttcctttctcgccacgt | |
| | tcgccggctttccccgtcaagctctaaatcgggggctccctttagggttccgatttagtgctttacg | |
| | gcacctcgaccccaaaaaacttgattagggtgatggttcacgtagtgggccatcgccctgatag | |
| | acggtttttcgccctttgacgttggagtccacgttctttaatagtggactcttgttccaaactggaac | |
| | aacactcaaccctatctcggtctattcttttgatttataagggattttgccgatttcggcctattggtta | |
| | aaaaatgagctgatttaacaaaaatttaacgc | |
| 7 | ATGTGTCCTCAGAAGCTAACCATCTCCTGGTTTGCCATCG | Signaling Peptide |
| | TTTTGCTGGTGTCTCCACTCATGGCC | |
| 8 | ATGTGGGAGCTGGAGAAAGACGTTTATGTTGTAGAGGTG | Mouse IL-12β |
| | GACTGGACTCCCGATGCCCCTGGAGAAACAGTGAACCTC | |
| | ACCTGTGACACGCCTGAAGAAGATGACATCACCTGGACC | |
| | TCAGACCAGAGACATGGAGTCATAGGCTCTGGAAAGACC | |
| | CTGACCATCACTGTCAAAGAGTTTCTAGATGCTGGCCAGT | |
| | ACACCTGCCACAAAGGAGGCGAGACTCTGAGCCACTCAC | |
| | ATCTGCTGCTCCACAAGAAGGAAAATGGAATTTGGTCCA | |
| | CTGAAATTTTAAAAAATTTCAAAAACAAGACTTTCCTGAA | |
| | GTGTGAAGCACCAAATTACTCCGGACGGTTCACGTGCTC | |
| | ATGGCTGGTGCAAAGAAACATGGACTTGAAGTTCAACAT | |
| | CAAGAGCAGTAGCAGTTCCCCTGACTCTCGGGCAGTGAC | |
| | ATGTGGAATGGCGTCTCTGTCTGCAGAGAAGGTCACACT | |
| | GGACCAAAGGGACTATGAGAAGTATTCAGTGTCCTGCCA | |
| | GGAGGATGTCACCTGCCCAACTGCCGAGGAGACCCTGCC | |
| | CATTGAACTGGCGTTGGAAGCACGGCAGCAGAATAAATA | |
| | TGAGAACTACAGCACCAGCTTCTTCATCAGGGACATCATC | |
| | AAACCAGACCCGCCCAAGAACTTGCAGATGAAGCCTTTG | |
| | AAGAACTCACAGGTGGAGGTCAGCTGGGAGTACCCTGAC | |
| | TCCTGGAGCACTCCCCATTCCTACTTCTCCCTCAAGTTCTT | |
| | TGTTCGAATCCAGCGCAAGAAAGAAAAGATGAAGGAGA | |
| | CAGAGGAGGGGTGTAACCAGAAAGGTGCGTTCCTCGTAG | |
| | AGAAGACATCTACCGAAGTCCAATGCAAAGGCGGGAATG | |
| | TCTGCGTGCAAGCTCAGGATCGCTATTACAATTCCTCATG | |
| | CAGCAAGTGGGCATGTGTTCCCTGCAGGGTCCGATCC | |
| 9 | GGCGGCGGCGGGAGTGGCGGCGGGGGTTCTGGCGGAGG | Linker |
| | CCTCGCTAGCGGTGG | |
| 10 | AGGGTCATTCCAGTCTCTGGACCTGCCAGGTGTCTTAGCC | Mouse IL-12α |
| | AGTCCCGAAACCTGCTGAAGACCACAGATGACATGGTGA | |
| | AGACGGCCAGAGAAAAACTGAAACATTATTCCTGCACTG | |
| | CTGAAGACATCGATCATGAAGACATCACACGGGACCAAA | |
| | CCAGCACATTGAAGACCTGTTTACCACTGGAACTACACA | |
| | AGAACGAGAGTTGCCTGGCTACTAGAGAGACTTCTTCCA | |
| | CAACAAGAGGGAGCTGCCTGCCCCCACAGAAGACGTCTT | |
| | TGATGATGACCCTGTGCCTTGGTAGCATCTATGAGGACTT | |
| | GAAGATGTACCAGACAGAGTTCCAGGCCATCAACGCAGC | |
| | ACTTCAGAATCACAACCATCAGCAGATCATTCTAGACAA | |
| | GGGCATGCTGGTGGCCATCGATGAGCTGATGCAGTCTCT | |
| | GAATCATAATGGCGAGACTCTGCGCCAGAAACCTCCTGT | |
| | GGGAGAAGCAGACCCTTACAGAGTGAAAATGAAGCTCTG | |
| | CATCCTGCTTCACGCCTTCAGCACCCGCGTCGTGACCATC | |
| | AACAGGGTGATGGGCTATCTGAGCTCCGCC | |
| 11 | tggaagggctaattcactcccaaagaagacaagatatccttgatctgtggatctaccacacacaa | Lenti-X ™ Tet- |
| | ggctacttccctgattagcagaactacacaccagggccaggggtcagatatccactgacctttg | One ™ Inducible |
| | gatggtgctacaagctagtaccagttgagccagataaggtagaagaggccaataaaggagag | Expression System |
| | aacaccagcttgttacaccctgtgagcctgcatgggatggatgacccggagagagaagtgtta | (Puro)(Takara Bio) |
| | gagtggaggtttgacagccgcctagcatttcatcacgtggcccgagagctgcatccggagtact | |
| | tcaagaactgctgatatcgagcttgctacaagggactttccgctggggactttccagggaggcg | |
| | tggcctgggcgggactggggagtggcgagccctcagatcctgcatataagcagctgctttttgc | |
| | ctgtactgggtctctctggttagaccagatctgagcctgggagctctctggctaactagggaacc | |
| | cactgcttaagcctcaataaagcttgccttgagtgcttcaagtagtgtgtgcccgtctgttgtgtga | |
| | ctctggtaactagagatccctcagacccttttagtcagtgtggaaaatctctagcagtggcgccc | |
| | gaacagggacttgaaagcgaaagggaaaccagaggagctctctcgacgcaggactcggctt | |
| | gctgaagcgcgcacggcaagaggcgaggggcggcgactggtgagtacgccaaaaattttga | |
| | ctagcggaggctagaaggagagagatgggtgcgagagcgtcagtattaagcgggggagaat | |
| | tagatcgcgatgggaaaaaattcggttaaggccagggggaaagaaaaaatataaattaaaaca | |
| | tatagtatgggcaagcagggagctagaacgattcgcagttaatcctggcctgttagaaacatca | |
| | gaaggctgtagacaaatactgggacagctacaaccatcccttcagacaggatcagaagaactt | |
| | agatcattatataatacagtagcaaccctctattgtgtgcatcaaaggatagagataaaagacac | |
| | caaggaagctttagacaagatagaggaagagcaaaacaaaagtaagaccaccgcacagcaa | |
| | gcggccggccgctgatcttcagacctggaggaggagatatgagggacaattggagaagtgaa | |
| | ttatataaatataaagtagtaaaaattgaaccattaggagtagcacccaccaaggcaaagagaa | |
| | gagtggtgcagagagaaaaaagagcagtgggaataggagctttgttccttgggttcttgggag | |
| | cagcaggaagcactatgggcgcagcgtcaatgacgctgacggtacaggccagacaattattgt | |
| | ctggtatagtgcagcagcagaacaatttgctgagggctattgaggcgcaacagcatctgttgca | |
| | actcacagtctggggcatcaagcagctccaggcaagaatcctggctgtggaaagatacctaaa | |
| | ggatcaacagctcctggggatttggggttgctctggaaaactcatttgcaccactgctgtgccttg | |
| | gaatgctagttggagtaataaatctctggaacagatttggaatcacacgacctggatggagtgg | |
| | gacagagaaattaacaattacacaagcttaatacactccttaattgaagaatcgcaaaaccagca | |
| | agaaaagaatgaacaagaattattggaattagataaatgggcaagtttgtggaattggtttaacat | |
| | aacaaattggctgtggtatataaaattattcataatgatagtaggaggcttggtaggtttaagaata | |
| | gtttttgctgtactttctatagtgaatagagttaggcagggatattcaccattatcgtttcagaccca | |
| | cctoccaaccccgaggggacccgacaggcccgaaggaatagaagaagaaggtggagaga | |
| | gagacagagacagatccattcgattagtgaacggatctcgacggtatcgcctttaaaagaaaag | |
| | gggggattggggggtacagtgcaggggaaagaatagtagacataatagcaacagacataca | |
| | aactaaagaattacaaaaacaaattacaaaaattcaaaattttcgggtttattacagggacagcag | |
| | agatccagtttatcgacttaacttgtttattgcagcttataatggttacaaataaggcaatagcatca | |
| | caaatttcacaaataaggcatttttttcactgcattctagttttggtttgtccaaactcatcaatgtatct | |
| | tatcatgtctggatctcaaatccctcggaagctgcgcctgtcttaggttggagtgatacatttttatc | |
| | acttttacccgtctttggattaggcagtagctctgacggccctcctgtcttaggttagtgaaaaatg | |
| | tcactctcttacccgtcattggctgtccagcttagctcgcaggggaggtggtctggatccgccgg | |
| | caccggtgtatacgggaattctttacgagggtaggaagtggtacggaaagttggtataagacaa | |
| | aagtgttgtggaattgaagtttactcaaaaaatcagcactcttttataggcgccctggtttacataa | |
| | gcaaagcttatacgttctctatcactgatagggagtaaactggatatacgttctctatcactgatag | |
| | ggagtaaactgtagatacgttctctatcactgatagggagtaaactggtcatacgttctctatcact | |
| | gatagggagtaaactccttatacgttctctatcactgatagggagtaaagtctgcatacgttctcta | |
| | tcactgatagggagtaaactcttcatacgttctctatcactgatagggagtaaactcgaggtgata | |
| | attccacggggttggggttgcgccttttccaaggcagccctgggtttgcgcagggacgcggct | |
| | gctctgggcgtggttccgggaaacgcagcggcgccgaccctgggtctcgcacattcttcacgt | |
| | ccgttcgcagcgtcacccggatcttcgccgctacccttgtgggccccccggcgacgcttcctgc | |
| | tccgcccctaagtcgggaaggttccttgcggttcgcggcgtgccggacgtgacaaacggaag | |
| | ccgcacgtctcactagtaccctcgcagacggacagcgccagggagcaatggcagcgcgccg | |
| | accgcgatgggctgtggccaatagcggctgctcagcagggcgcgccgagagcagcggccg | |
| | ggaaggggcggtgcgggaggcggggtgtggggcggtagtgtgggccctgttcctgcccgc | |
| | gcggtgttccgcattctgcaagcctccggagcgcacgtcggcagtcggctccctcgttgaccg | |
| | aatcaccgacctctctccccagggggatcatcgaattaccatgtctagactggacaagagcaaa | |
| | gtcataaactctgctctggaattactcaatggagtcggtatcgaaggcctgacgacaaggaaac | |
| | tcgctcaaaagctgggagttgagcagcctaccctgtactggcacgtgaagaacaagcgggcc | |
| | ctgctcgatgccctgccaatcgagatgctggacaggcatcatacccactcctgccccctggaa | |
| | ggcgagtcatggcaagactttctgcggaacaacgccaagtcataccgctgtgctctcctctcac | |
| | atcgcgacggggctaaagtgcatctcggcacccgcccaacagagaaacagtacgaaaccct | |
| | ggaaaatcagctcgcgttcctgtgtcagcaaggcttctccctggagaacgcactgtacgctctgt | |
| | ccgccgtgggccactttacactgggctgcgtattggaggaacaggagcatcaagtagcaaaa | |
| | gaggaaagagagacacctaccaccgattctatgcccccacttctgaaacaagcaattgagctgt | |
| | tcgaccggcagggagccgaacctgccttccttttcggcctggaactaatcatatgtggcctgga | |
| | gaaacagctaaagtgcgaaagcgggggccgaccgacgcccttgacgattttgacttagacat | |
| | gctcccagccgatgcccttgacgactttgaccttgatatgctgcctgctgacgctcttgacgatttt | |
| | gaccttgacatgctccccgggtaaacgcgcgaatgtgtgtcagttagggtgtggaaagtcccca | |
| | ggctccccagcaggcagaagtatgcaaagcatgcatctcaattagtcagcaaccaggtgtgga | |
| | aagtccccaggctccccagcaggcagaagtatgcaaagcatgcatctcaattagtcagcaacc | |
| | atagtcccgcccctaactccgcccatccegcccctaactcegcccagttccgcccattctccgc | |
| | cccatggctgactaattttttttatttatgcagaggccgaggccgcctcggcctctgagctattcca | |
| | gaagtagtgaggaggcttttttggaggcctaggcttttgcaaaacgcgaccatgaccgagtaca | |
| | agcccacggtgcgcctcgccacccgcgacgacgtcccccgggccgtacgcaccctegccgc | |
| | cgcgttcgccgactaccccgccacgcgccacacegtcgacccggaccgccacatcgagcgg | |
| | gtcaccgagctgcaagaactcttcctcacgcgcgtcgggctcgacatcggcaaggtgtgggtc | |
| | gcggacgacggcgccgcggtggcggtctggaccacgccggagagcgtcgaagcgggggc | |
| | ggtgttcgccgagatcggcccgcgcatggccgagttgagcggttcccggctggccgcgcag | |
| | caacagatggaaggcctcctggcgccgcaccggcccaaggagcccgcgtggttcctggcca | |
| | ccgtcggcgtctcgcccgaccaccagggcaagggtctgggcagcgccgtcgtgctccccgg | |
| | agtggaggcggccgagcgcgccggggtgcccgccttcctggagacctccgcgccccgcaa | |
| | cctccccttctacgagcggctcggcttcaccgtcaccgccgacgtcgaggtgcccgaaggac | |
| | cgcgcacctggtgcatgacccgcaagcccggtgcctgaacgcgtctggaacaatcaacctct | |
| | ggattacaaaatttgtgaaagattgactggtattcttaactatgttgctccttttacgctatgtggata | |
| | cgctgctttaatgcctttgtatcatgctattgcttcccgtatggctttcattttctcctccttgtataaatc | |
| | ctggttgctgtctctttatgaggagttgtggcccgttgtcaggcaacgtggcgtggtgtgcactgt | |
| | gtttgctgacgcaacccccactggttggggcattgccaccacctgtcagctcctttccgggacttt | |
| | cgctttccccctccctattgccacggcggaactcategccgcctgccttgcccgctgctggaca | |
| | ggggctcggctgttgggcactgacaattccgtggtgttgtcggggaagctgacgtcctttccatg | |
| | gctgctcgcctgtgttgccacctggattctgcgcgggacgtccttctgctacgtcccttcggccct | |
| | caatccagcggaccttccttcccgcggcctgctgccggctctgcggcctettccgcgtcttcgc | |
| | cttcgccctcagacgagtcggatctccctttgggccgcctccccgcctggaattaattctgcagt | |
| | cgagacctagaaaaacatggagcaatcacaagtagcaatacagcagctaccaatgctgattgt | |
| | gcctggctagaagcacaagaggaggaggaggtgggttttccagtcacacctcaggtaccttta | |
| | agaccaatgacttacaaggcagctgtagatcttagccactttttaaaagaaaagaggggactgg | |
| | aagggctaattcactcccaacgaagacaagatatccttgatctgtggatctaccacacacaagg | |
| | ctacttccctgattagcagaactacacaccagggccaggggtcagatatccactgacctttggat | |
| | ggtgctacaagctagtaccagttgagccagataaggtagaagaggccaataaaggagagaac | |
| | accagcttgttacaccctgtgagcctgcatgggatggatgacccggagagagaagtgttagag | |
| | tggaggtttgacagccgcctagcatttcatcacgtggcccgagagctgcatccggagtacttca | |
| | agaactgctgatatcgagcttgctacaagggactttccgctggggactttccagggaggcgtgg | |
| | cctgggcgggactggggagtggcgagccctcagatcctgcatataagcagctgctttttgcctg | |
| | tactgggtctctctggttagaccagatctgagcctgggagctctctggctaactagggaacccac | |
| | tgcttaagcctcaataaagcttgccttgagtgcttcaagtagtgtgtgcccgtctgttgtgtgactct | |
| | ggtaactagagatccctcagacccttttagtcagtgtggaaaatctctagcagtagtagttcatgt | |
| | catcttattattcagtatttataacttgcaaagaaatgaatatcagagagtgagaggccttgacatt | |
| | gctagcgtttaccgtcgacctctagctagagcttggcgtaatcatggtcatagctgtttcctgtgtg | |
| | aaattgttatccgctcacaattccacacaacatacgagccggaagcataaagtgtaaagcctgg | |
| | ggtgcctaatgagtgagctaactcacattaattgcgttgcgctcactgcccgctttccagtcggg | |
| | aaacctgtcgtgccagctgcattaatgaatcggccaacgcgcggggagaggcggtttgcgtat | |
| | tgggcgctcttccgcttcctcgctcactgactcgctgcgctcggtcgttcggctgcggcgagcg | |
| | gtatcagctcactcaaaggcggtaatacggttatccacagaatcaggggataacgcaggaaag | |
| | aacatgtgagcaaaaggccagcaaaaggccaggaaccgtaaaaaggccgcgttgctggcgt | |
| | ttttccataggctccgcccccctgacgagcatcacaaaaatcgacgctcaagtcagaggtggc | |
| | gaaacccgacaggactataaagataccaggcgtttccccctggaagctccctegtgcgctctcc | |
| | tgttccgaccctgccgcttaccggatacctgtccgcctttctcccttcgggaagcgtggcgctttc | |
| | tcatagctcacgctgtaggtatctcagttcggtgtaggtcgttcgctccaagctgggctgtgtgca | |
| | cgaaccccccgttcagcccgaccgctgcgccttatccggtaactatcgtcttgagtccaacccg | |
| | gtaagacacgacttatcgccactggcagcagccactggtaacaggattagcagagcgaggtat | |
| | gtaggcggtgctacagagttcttgaagtggtggcctaactacggctacactagaagaacagtat | |
| | ttggtatctgcgctctgctgaagccagttaccttcggaaaaagagttggtagctcttgatccggca | |
| | aacaaaccaccgctggtagcggtggtttttttgtttgcaagcagcagattacgcgcagaaaaaa | |
| | aggatctcaagaagatcctttgatcttttctacggggtctgacgctcagtggaacgaaaactcac | |
| | gttaagggattttggtcatgagattatcaaaaaggatcttcacctagatccttttaaattaaaaatga | |
| | agttttaaatcaatctaaagtatatatgagtaaacttggtctgacagttaccaatgcttaatcagtga | |
| | ggcacctatctcagcgatctgtctatttcgttcatccatagttgcctgactccccgtcgtgtagata | |
| | actacgatacgggagggcttaccatctggccccagtgctgcaatgataccgcgagacccacg | |
| | ctcaccggctccagatttatcagcaataaaccagccagccggaagggccgagcgcagaagtg | |
| | gtcctgcaactttatccgcctccatccagtctattaattgttgccgggaagctagagtaagtagttc | |
| | gccagttaatagtttgcgcaacgttgttgccattgctacaggcatcgtggtgtcacgctcgtcgttt | |
| | ggtatggcttcattcagctccggttcccaacgatcaaggcgagttacatgatcccccatgttgtgc | |
| | aaaaaagcggttagctccttcggtcctccgatcgttgtcagaagtaagttggccgcagtgttatc | |
| | actcatggttatggcagcactgcataattctcttactgtcatgccatccgtaagatgcttttctgtga | |
| | ctggtgagtactcaaccaagtcattctgagaatagtgtatgcggcgaccgagttgctcttgcccg | |
| | gcgtcaatacgggataataccgcgccacatagcagaactttaaaagtgctcatcattggaaaac | |
| | gttcttcggggcgaaaactctcaaggatcttaccgctgttgagatccagttcgatgtaacccactc | |
| | gtgcacccaactgatcttcagcatcttttactttcaccagcgtttctgggtgagcaaaaacaggaa | |
| | ggcaaaatgccgcaaaaaagggaataagggcgacacggaaatgttgaatactcatactcttcc | |
| | tttttcaatattattgaagcatttatcagggttattgtctcatgagcggatacatatttgaatgtatttag | |
| | aaaaataaacaaataggggttccgcgcacatttccccgaaaagtgccacctgacgtcgacgga | |
| | tcgggagatcaacttgtttattgcagcttataatggttacaaataaagcaatagcatcacaaatttc | |
| | acaaataaagcatttttttcactgcattctagttgtggtttgtccaaactcatcaatgtatcttatcatgt | |
| | ctggatcaactggataactcaagctaaccaaaatcatcccaaacttcccaccccataccctatta | |
| | ccactgccaattacctgtggtttcatttactctaaacctgtgattcctctgaattattttcattttaaaga | |
| | aattgtatttgttaaatatgtactacaaacttagtagt | |
| 12 | AAGCTGGGTGCCCCGGAGAGTCTGACCACC | Pre-CDS #2 |
| 13 | ATGCCACCTCCTCGCCTCCTCTTCTTCCTCCTCTTCCTCAC | Secretion Signal |
| | CCCCATGGAAGTCAGGCCC | |
| 14 | GAGGAACCTCTAGTGGTGAAGGTGGAAGAGGGAGATAA | human CD-19t |
| | CGCTGTGCTGCAGTGCCTCAAGGGGACCTCAGATGGCCC | |
| | CACTCAGCAGCTGACCTGGTCTCGGGAGTCCCCGCTTAAA | |
| | CCCTTCTTAAAACTCAGCCTGGGCCTGCCAGGCCTGGGAA | |
| | TCCACATGAGGCCCCTGGCCATCTGGCTTTTCATCTTCAA | |
| | CGTCTCTCAACAGATGGGGGGCTTCTACCTGTGCCAGCCA | |
| | GGGCCCCCCTCTGAGAAGGCCTGGCAGCCTGGCTGGACA | |
| | GTCAATGTGGAGGGCAGCGGGGAGCTGTTCCGGTGGAAT | |
| | GTTTCGGACCTAGGTGGCCTGGGCTGTGGCCTGAAGAAC | |
| | AGGTCCTCAGAGGGCCCCAGCTCCCCTTCCGGGAAGCTC | |
| | ATGAGCCCCAAGCTGTATGTGTGGGCCAAAGACCGCCCT | |
| | GAGATCTGGGAGGGAGAGCCTCCATGTGGCCCACCGAGG | |
| | GACAGCCTGAACCAGAGCCTCAGCCAGGACCTCACCATG | |
| | GCCCCTGGCTCCACACTCTGGCTGTCCTGTGGGGTACCCC | |
| | CTGACTCTGTATCCAGGGGCCCCCTCTCCTGGACCCATGT | |
| | GCACCCCAAGGGGCCTAAGTCATTGCTGAGCCTAGAGCT | |
| | GAAGGACGATCGCCCGGCCAGAGATATGTGGGTAATGGA | |
| | GACGGGTCTGTTGTTGCCCCGGGCCACAGCTCAAGATGCT | |
| | GGAAAGTATTATTGTCACCGTGGCAACCTGACCATGTCAT | |
| | TCCACCTGGAGATCACTGCTCGGCCAGTACTATGGCACTG | |
| | GCTGCTGAGGACTGGTGGCTGGAAGGTCTCAGCTGTGAC | |
| | TTTGGCTTATCTGATCTTCTGC | |
| 15 | AGAAGAAACAACATCTGTTTCAGGGCCATTGGACTCTCC | Signaling Peptide |
| | GTCCTGCCCAGAGCAAG | |
| 16 | ATATGGGAACTGAAGAAAGATGTTTATGTCGTAGAATTG | human IL-12β |
| | GATTGGTATCCGGATGCCCCTGGAGAAATGGTGGTCCTC | |
| | ACCTGTGACACCCCTGAAGAAGATGGTATCACCTGGACC | |
| | TTGGACCAGAGCAGTGAGGTCTTAGGCTCTGGCAAAACC | |
| | CTGACCATCCAAGTCAAAGAGTTTGGAGATGCTGGCCAG | |
| | TACACCTGTCACAAAGGAGGCGAGGTTCTAAGCCATTCG | |
| | CTCCTGCTGCTTCACAAAAAGGAAGATGGAATTTGGTCC | |
| | ACTGATATTTTAAAGGACCAGAAAGAACCCAAAAATAAG | |
| | ACCTTTCTAAGATGCGAGGCCAAGAATTATTCTGGACGTT | |
| | TCACCTGCTGGTGGCTGACGACAATCAGTACTGATTTGAC | |
| | ATTCAGTGTCAAAAGCAGCAGAGGCTCTTCTGACCCCCA | |
| | AGGGGTGACGTGCGGAGCTGCTACACTCTCTGTAGAGAG | |
| | AGTCAGAGGGGACAACAAGGAGTATGAGTACTCAGTGGA | |
| | GTGCCAGGAGGACAGTGCCTGCCCAGCTGCTGAGGAGAG | |
| | TCTGCCCATTGAGGTCATGGTGGATGCCGTTCACAAGCTC | |
| | AAGTATGAAAACTACACCAGCAGCTTCTTCATCAGGGAC | |
| | ATCATCAAACCTGACCCACCCAAGAACTTGCAGCTGAAG | |
| | CCATTAAAGAATTCTCGGCAGGTGGAGGTCAGCTGGGAG | |
| | TACCCTGACACCTGGAGTACTCCACATTCCTACTTCTCCC | |
| | TGACATTCTGCGTTCAGGTCCAGGGCAAGAGCAAGAGAG | |
| | AAAAGAAAGATAGAGTCTTCACGGACAAGACCTCAGCCA | |
| | CGGTCATCTGCCGCAAAAATGCCAGCATTAGCGTGCGGG | |
| | CCCAGGACCGCTACTATAGCTCATCTTGGAGCGAATGGG | |
| | CATCTGTGCCCTGCAGT | |
| 17 | AGAAACCTCCCCGTGGCCACTCCAGACCCAGGAATGTTC | human IL-12α |
| | CCATGCCTTCACCACTCCCAAAACCTGCTGAGGGCCGTCA | |
| | GCAACATGCTCCAGAAGGCCAGACAAACTCTAGAATTTT | |
| | ACCCTTGCACTTCTGAAGAGATTGATCATGAAGATATCAC | |
| | AAAAGATAAAACCAGCACAGTGGAGGCCTGTTTACCATT | |
| | GGAATTAACCAAGAATGAGAGTTGCCTAAATTCCAGAGA | |
| | GACCTCTTTCATAACTAATGGGAGTTGCCTGGCCTCCAGA | |
| | AAGACCTCTTTTATGATGGCCCTGTGCCTTAGTAGTATTT | |
| | ATGAAGACTTGAAGATGTACCAGGTGGAGTTCAAGACCA | |
| | TGAATGCAAAGCTTCTGATGGATCCTAAGAGGCAGATCT | |
| | TTCTAGATCAAAACATGCTGGCAGTTATTGATGAGCTGAT | |
| | GCAGGCCCTGAATTTCAACAGTGAGACTGTGCCACAAAA | |
| | ATCCTCCCTTGAAGAACCGGATTTTTATAAAACTAAAATC | |
| | AAGCTCTGCATACTTCTTCATGCTTTCAGAATTCGGGCAG | |
| | TGACTATTGATAGAGTGATGAGCTATCTGAATGCTTCC | |
| 18 | gagagagagacagagacagatccattcgattagtgaacggatctcgacggtatcggttaactttt | Mouse CD19- |
| | aaaagaaaaggggggattggggggtacagtgcaggggaaagaatagtagacataatagcaa | P2A-HSV TK-Neo |
| | cagacatacaaactaaagaattacaaaaacaaattacaaaattcaaaattttatcgataagcttgg | |
| | gagttccgcgttacataacttacggtaaatggcccgcctggctgaccgcccaacgacccccgc | |
| | ccattgacgtcaataatgacgtatgttcccatagtaacgccaatagggactttccattgacgtcaa | |
| | tgggtggagtatttacggtaaactgcccacttggcagtacatcaagtgtatcatatgccaagtac | |
| | gccccctattgacgtcaatgacggtaaatggcccgcctggcattatgcccagtacatgaccttat | |
| | gggactttcctacttggcagtacatctacgtattagtcatcgctattaccatggtgatgcggttttgg | |
| | cagtacatcaatgggcgtggatagcggtttgactcacggggatttccaagtctccaccccattga | |
| | cgtcaatgggagtttgttttggcaccaaaatcaacgggactttccaaaatgtcgtaacaactccg | |
| | ccccattgacgcaaatgggcggtaggcgtgtacggtgggaggtctatataagcagagctcgttt | |
| | agtgaaccgtcagatcgcctggagacgccatccacgctgttttgacctccatagaagacaccg | |
| | actctagaGACACCACGCAGAAGCCGGGTGCCCCAGGGAGCC | |
| | TGGCTACCATGCCATCTCCTCTCCCTGTCTCCTTCCTCCTC | |
| | TTTCTTACCTTAGTAGGAGGCAGGCCCCAGAAGTCCTTAC | |
| | TGGTGGAGGTAGAAGAGGGAGGCAATGTTGTGCTGCCAT | |
| | GCCTCCCGGACTCCTCACCTGTCTCTTCTGAGAAGCTGGC | |
| | TTGGTATCGAGGTAACCAGTCAACACCCTTCCTGGAGCTG | |
| | AGCCCCGGGTCCCCTGGCCTGGGATTGCACGTGGGGTCC | |
| | CTGGGCATCTTGCTAGTGATTGTCAATGTCTCAGACCATA | |
| | TGGGGGGCTTCTACCTGTGCCAGAAGAGGCCCCCTTTCAA | |
| | GGACATCTGGCAGCCTGCCTGGACAGTGAACGTGGAGGA | |
| | TAGTGGGGAGATGTTCCGGTGGAATGCTTCAGACGTCAG | |
| | GGACCTGGACTGTGACCTAAGGAACAGGTCCTCTGGGAG | |
| | CCACAGGTCCACTTCTGGTTCCCAGCTGTATGTGTGGGCT | |
| | AAAGACCATCCTAAGGTCTGGGGAACAAAGCCTGTATGT | |
| | GCCCCTCGGGGGAGCAGTTTGAATCAGAGTCTAATCAAC | |
| | CAAGATCTCACTGTGGCACCCGGCTCCACACTTTGGCTGT | |
| | CCTGTGGGGTACCCCCTGTCCCAGTGGCCAAAGGCTCCAT | |
| | CTCCTGGACCCATGTGCATCCTAGGAGACCTAATGTTTCA | |
| | CTACTGAGCCTAAGCCTTGGGGGAGAGCACCCGGTCAGA | |
| | GAGATGTGGGTTTGGGGGTCTCTTCTGCTTCTGCCCCAAG | |
| | CCACAGCTTTAGATGAAGGCACCTATTATTGTCTCCGAGG | |
| | AAACCTGACCATCGAGAGGCACGTGAAGGTCATTGCAAG | |
| | GTCAGTGTGGCTCTGGCTGTTGAGAACTGGTGGATGGAT | |
| | AGTCCCAGTGGTGACTTTAGTATATGTCATCTTCggatccaccg | |
| | gtcgccaccatggtgagcaagggcgaggagctgttcaccggggtggtgcccatcctggtcga | |
| | gctggacggcgacgtaaacggccacaagttcagcgtgtccggcgagggcgagggcgatgc | |
| | cacctacggcaagctgaccctgaagttcatctgcaccaccggcaagctgcccgtgccctggcc | |
| | caccctcgtgaccaccctgacctacggcgtgcagtgcttcagccgctaccccgaccacatgaa | |
| | gcagcacgacttcttcaagtccgccatgcccgaaggctacgtccaggagcgcaccatcttcttc | |
| | aaggacgacggcaactacaagacccgcgccgaggtgaagttcgagggcgacaccctggtg | |
| | aaccgcatcgagctgaagggcatcgacttcaaggaggacggcaacatcctggggcacaagc | |
| | tggagtacaactacaacagccacaacgtctatatcatggccgacaagcagaagaacggcatca | |
| | aggtgaacttcaagatccgccacaacatcgaggacggcagcgtgcagctcgccgaccactac | |
| | cagcagaacacccccatcggcgacggccccgtgctgctgcccgacaaccactacctgagca | |
| | cccagtccgccctgagcaaagaccccaacgagaagcgcgatcacatggtcctgctggagttc | |
| | gtgaccgccgccgggatcactctcggcatggacgagctgtacaagtaaagcggccgcgtcga | |
| | caatcaacctctggattacaaaatttgtgaaagattgactggtattcttaactatgttgctccttttac | |
| | gctatgtggatacgctgctttaatgcctttgtatcatgctattgcttcccgtatggctttcattttctcct | |
| | ccttgtataaatcctggttgctgtctctttatgaggagttgtggcccgttgtcaggcaacgtggcgt | |
| | ggtgtgcactgtgtttgctgacgcaacccccactggttggggcattgccaccacctgtcagctcc | |
| | tttccgggactttcgctttccccctccctattgccacggcggaactcategccgcctgccttgccc | |
| | gctgctggacaggggctcggctgttgggcactgacaattccgtggtgttgtcggggaagctga | |
| | cgtcctttccatggctgctcgcctgtgttgccacctggattctgcgcgggacgtccttctgctacg | |
| | tcccttcggccctcaatccagcggaccttccttcccgcggcctgctgccggctctgcggcctctt | |
| | ccgcgtcttcgccttcgccctcagacgagtcggatctccctttgggccgcctccccgcctggaa | |
| | ttctgcagatatccggactagtgatctaattctaccgggtaggggaggcgcttttcccaaggcag | |
| | tctggagcatgcgctttagcagccccgctgggcacttggcgctacacaagtggcctctggcctc | |
| | gcacacattccacatccaccggtaggcgccaaccggctccgttctttggtggccccttcgcgcc | |
| | accttctactcctcccctagtcaggaagttcccccccgccccgcagctcgcgtcgtgcaggacg | |
| | tgacaaatggaagtagcacgtctcactagtctcgtgcagatggacagcaccgctgagcaatgg | |
| | aagcgggtaggcctttggggcagcggccaatagcagctttgctccttcgctttctgggctcaga | |
| | ggctgggaagggggggtccgggggcgggctcaggggcgggctcaggggcggggcggg | |
| | cgcccgaaggtcctccggaggcccggcattctgcacgcttcaaaagcgcacgtctgccgcgc | |
| | tgttctcctcttcctcatctccgggcctttcgacctgcagccaatatgggatcggccattgaacaa | |
| | gatggattgcacgcaggttctccggccgcttgggtggagaggctattcggctatgactgggca | |
| | caacagacaatcggctgctctgatgccgccgtgttccggctgtcagcgcaggggcgcccggtt | |
| | ctttttgtcaagaccgacctgtccggtgccctgaatgaactgcaggacgaggcagcgcggctat | |
| | cgtggctggccacgacgggcgttccttgcgcagctgtgctcgacgttgtcactgaagcgggaa | |
| | gggactggctgctattgggcgaagtgccggggcaggatctcctgtcatctcaccttgctcctgc | |
| | cgagaaagtatccatcatggctgatgcaatgcggcggctgcatacgcttgatccggctacctgc | |
| | ccattcgaccaccaagcgaaacatcgcatcgagcgagcacgtactcggatggaagccggtctt | |
| | gtcgatcaggatgatctggacgaagagcatcaggggctcgcgccagccgaactgttcgccag | |
| | gctcaaggcgcgcatgcccgacggcgaggatctcgtcgtgacccatggcgatgcctgcttgc | |
| | cgaatatcatggtggaaaatggccgcttttctggattcatcgactgtggccggctgggtgtggcg | |
| | gaccgctatcaggacatagcgttggctacccgtgatattgctgaagagcttggcggcgaatgg | |
| | gctgaccgcttcctcgtgctttacggtatcgccgctcccgattcgcagcgcatcgccttctatcgc | |
| | cttcttgacgagttcttctgaggggatccGCCACCAACTTCTCCCTGCTGAA | |
| | GCAGGCCGGCGACGTGGAGGAGAACCCCGGCCCCGCTTC | |
| | GTACCCCGGCCATCAACACGCGTCTGCGTTCGACCAGGCT | |
| | GCGCGTTCTCGCGGCCATAGCAACCGACGTACGGCGTTG | |
| | CGCCCTCGCCGGCAGCAAGAAGCCACGGAAGTCCGCCCG | |
| | GAGCAGAAAATGCCCACGCTACTGCGGGTTTATATAGAC | |
| | GGTCCCCACGGGATGGGGAAAACCACCACCACGCAACTG | |
| | CTGGTGGCCCTGGGTTCGCGCGACGATATCGTCTACGTAC | |
| | CCGAGCCGATGACTTACTGGCGGGTGCTGGGGGCTTCCG | |
| | AGACAATCGCGAACATCTACACCACACAACACCGCCTCG | |
| | ACCAGGGTGAGATATCGGCCGGGGACGCGGCGGTGGTAA | |
| | TGACAAGCGCCCAGATAACAATGGGCATGCCTTATGCCG | |
| | TGACCGACGCCGTTCTGGCTCCTCATATCGGGGGGGAGG | |
| | CTGGGAGCTCACATGCCCCGCCCCCGGCCCTCACCCTCAT | |
| | CTTCGACCGCCATCCCATCGCCGCCCTCCTGTGCTACCCG | |
| | GCCGCGCGGTACCTTATGGGCAGCATGACCCCCCAGGCC | |
| | GTGCTGGCGTTCGTGGCCCTCATCCCGCCGACCTTGCCCG | |
| | GCACCAACATCGTGCTTGGGGCCCTTCCGGAGGACAGAC | |
| | ACATCGACCGCCTGGCCAAACGCCAGCGCCCCGGCGAGC | |
| | GGCTGGACCTGGCTATGCTGGCTGCGATTCGCCGCGTTTA | |
| | CGGGCTACTTGCCAATACGGTGCGGTATCTGCAGTGCGG | |
| | CGGGTCGTGGCGGGAGGACTGGGGACAGCTTTCGGGGAC | |
| | GGCCGTGCCGCCCCAGGGTGCCGAGCCCCAGAGCAACGC | |
| | GGGCCCACGACCCCATATCGGGGACACGTTATTTACCCTG | |
| | TTTCGGGCCCCCGAGTTGCTGGCCCCCAACGGCGACCTGT | |
| | ATAACGTGTTTGCCTGGGCCTTGGACGTCTTGGCCAAACG | |
| | CCTCCGTTCCATGCACGTCTTTATCCTGGATTACGACCAA | |
| | TCGCCCGCCGGCTGCCGGGACGCCCTGCTGCAACTTACCT | |
| | CCGGGATGGTCCAGACCCACGTCACCACCCCCGGCTCCA | |
| | TACCGACGATATGCGACCTGGCGCGCACGTTTGCCCGGG | |
| | AGATGGGGGAGGCTAACTgggcccggtacctttaagaccaatgacttacaag | |
| | gcagctgtagatcttagccactttttaaaagaaaaggggggactggaagggctagctcactccc | |
| | aacgaagacaagatctgctttttgcttgtactgggtctctctggttagaccagatctgagcctggg | |
| | agctctctggctaactagggaacccactgcttaagcctcaataaagcttgccttgagtgcttcaa | |
| | gtagtgtgtgcccgtctgttgtgtgactctggtaactagagatccctcagacccttttagtcagtgt | |
| | ggaaaatctctagcagtagtagttcatgtcatcttattattcagtatttataacttgcaaagaaatga | |
| | atatcagagagtgagaggaacttgtttattgcagcttataatggttacaaataaagcaatagcatc | |
| | acaaatttcacaaataaagcatttttttcactgcattctagttgtggtttgtccaaactcatcaatgtat | |
| | cttatcatgtctggctctagctatcccgcccctaactcegcccatcccgcccctaactccgccca | |
| | gttccgcccattctccgccccatggctgactaattttttttatttatgcagaggccgaggccgcctc | |
| | ggcctctgagctattccagaagtagtgaggaggcttttttggaggcctagggacgtacccaattc | |
| | gccctatagtgagtcgtattacgcgcgctcactggccgtcgttttacaacgtcgtgactgggaaa | |
| | accctggcgttacccaacttaatcgccttgcagcacatccccctttcgccagctggcgtaatagc | |
| | gaagaggcccgcaccgatcgcccttcccaacagttgcgcagcctgaatggcgaatgggacg | |
| | cgccctgtagcggcgcattaagcgcggcgggtgtggtggttacgcgcagcgtgaccgctaca | |
| | cttgccagcgccctagcgcccgctcctttcgctttcttcccttcctttctcgccacgttcgccggctt | |
| | tccccgtcaagctctaaatcgggggctccctttagggttccgatttagtgctttacggcacctcga | |
| | ccccaaaaaacttgattagggtgatggttcacgtagtgggccatcgccctgatagacggtttttc | |
| | gccctttgacgttggagtccacgttctttaatagtggactcttgttccaaactggaacaacactcaa | |
| | ccctatctcggtctattcttttgatttataagggattttgccgatttcggcctattggttaaaaaatgag | |
| | ctgatttaacaaaaatttaacgc | |
| 19 | tggaagggctaattcactcccaaagaagacaagatatccttgatctgtggatctaccacacacaa | Tet Inducible |
| | ggctacttccctgattagcagaactacacaccagggccaggggtcagatatccactgacctttg | Mouse IL-12 |
| | gatggtgctacaagctagtaccagttgagccagataaggtagaagaggccaataaaggagag | PuroR |
| | aacaccagcttgttacaccctgtgagcctgcatgggatggatgacccggagagagaagtgtta | |
| | gagtggaggtttgacagccgcctagcatttcatcacgtggcccgagagctgcatccggagtact | |
| | tcaagaactgctgatatcgagcttgctacaagggactttccgctggggactttccagggaggcg | |
| | tggcctgggcgggactggggagtggcgagccctcagatcctgcatataagcagctgctttttgc | |
| | ctgtactgggtctctctggttagaccagatctgagcctgggagctctctggctaactagggaacc | |
| | cactgcttaagcctcaataaagcttgccttgagtgcttcaagtagtgtgtgcccgtctgttgtgtga | |
| | ctctggtaactagagatccctcagacccttttagtcagtgtggaaaatctctagcagtggcgccc | |
| | gaacagggacttgaaagcgaaagggaaaccagaggagctctctcgacgcaggactcggctt | |
| | gctgaagcgcgcacggcaagaggcgaggggcggcgactggtgagtacgccaaaaattttga | |
| | ctagcggaggctagaaggagagagatgggtgcgagagcgtcagtattaagcgggggagaat | |
| | tagatcgcgatgggaaaaaattcggttaaggccagggggaaagaaaaaatataaattaaaaca | |
| | tatagtatgggcaagcagggagctagaacgattcgcagttaatcctggcctgttagaaacatca | |
| | gaaggctgtagacaaatactgggacagctacaaccatcccttcagacaggatcagaagaactt | |
| | agatcattatataatacagtagcaaccctctattgtgtgcatcaaaggatagagataaaagacac | |
| | caaggaagctttagacaagatagaggaagagcaaaacaaaagtaagaccaccgcacagcaa | |
| | gcggccggccgctgatcttcagacctggaggaggagatatgagggacaattggagaagtgaa | |
| | ttatataaatataaagtagtaaaaattgaaccattaggagtagcacccaccaaggcaaagagaa | |
| | gagtggtgcagagagaaaaaagagcagtgggaataggagctttgttccttgggttcttgggag | |
| | cagcaggaagcactatgggcgcagcgtcaatgacgctgacggtacaggccagacaattattgt | |
| | ctggtatagtgcagcagcagaacaatttgctgagggctattgaggcgcaacagcatctgttgca | |
| | actcacagtctggggcatcaagcagctccaggcaagaatcctggctgtggaaagatacctaaa | |
| | ggatcaacagctcctggggatttggggttgctctggaaaactcatttgcaccactgctgtgccttg | |
| | gaatgctagttggagtaataaatctctggaacagatttggaatcacacgacctggatggagtgg | |
| | gacagagaaattaacaattacacaagcttaatacactccttaattgaagaatcgcaaaaccagca | |
| | agaaaagaatgaacaagaattattggaattagataaatgggcaagtttgtggaattggtttaacat | |
| | aacaaattggctgtggtatataaaattattcataatgatagtaggaggcttggtaggtttaagaata | |
| | gtttttgctgtactttctatagtgaatagagttaggcagggatattcaccattatcgtttcagaccca | |
| | cctcccaaccccgaggggacccgacaggcccgaaggaatagaagaagaaggtggagaga | |
| | gagacagagacagatccattcgattagtgaacggatctcgacggtatcgcctttaaaagaaaag | |
| | gggggattggggggtacagtgcaggggaaagaatagtagacataatagcaacagacataca | |
| | aactaaagaattacaaaaacaaattacaaaaattcaaaattttcgggtttattacagggacagcag | |
| | agatccagtttatcgacttaacttgtttattgcagcttataatggttacaaataaggcaatagcatca | |
| | caaatttcacaaataaggcatttttttcactgcattctagttttggtttgtccaaactcatcaatgtatct | |
| | tatcatgtctggatctcaaatccctcggaagctgcgcctgtcttaggttggagtgatacatttttatc | |
| | acttttacccgtctttggattaggcagtagctctgacggccctcctgtcttaggttagtgaaaaatg | |
| | tcactctcttacccgtcattggctgtccagcttagctcgcaggggaggtggtctggatccGGC | |
| | GGAGCTCAGATAGCCCATCACCCTGTTGATGGTCACGAC | |
| | GCGGGTGCTGAAGGCGTGAAGCAGGATGCAGAGCTTCAT | |
| | TTTCACTCTGTAAGGGTCTGCTTCTCCCACAGGAGGTTTC | |
| | TGGCGCAGAGTCTCGCCATTATGATTCAGAGACTGCATCA | |
| | GCTCATCGATGGCCACCAGCATGCCCTTGTCTAGAATGAT | |
| | CTGCTGATGGTTGTGATTCTGAAGTGCTGCGTTGATGGCC | |
| | TGGAACTCTGTCTGGTACATCTTCAAGTCCTCATAGATGC | |
| | TACCAAGGCACAGGGTCATCATCAAAGACGTCTTCTGTG | |
| | GGGGCAGGCAGCTCCCTCTTGTTGTGGAAGAAGTCTCTCT | |
| | AGTAGCCAGGCAACTCTCGTTCTTGTGTAGTTCCAGTGGT | |
| | AAACAGGTCTTCAATGTGCTGGTTTGGTCCCGTGTGATGT | |
| | CTTCATGATCGATGTCTTCAGCAGTGCAGGAATAATGTTT | |
| | CAGTTTTTCTCTGGCCGTCTTCACCATGTCATCTGTGGTCT | |
| | TCAGCAGGTTTCGGGACTGGCTAAGACACCTGGCAGGTC | |
| | CAGAGACTGGAATGACCCTCCACCGCTAGCGAGGCCTCC | |
| | GCCAGAACCCCCGCCGCCACTCCCGCCGCCGCCGGATCG | |
| | GACCCTGCAGGGAACACATGCCCACTTGCTGCATGAGGA | |
| | ATTGTAATAGCGATCCTGAGCTTGCACGCAGACATTCCCG | |
| | CCTTTGCATTGGACTTCGGTAGATGTCTTCTCTACGAGGA | |
| | ACGCACCTTTCTGGTTACACCCCTCCTCTGTCTCCTTCATC | |
| | TTTTCTTTCTTGCGCTGGATTCGAACAAAGAACTTGAGGG | |
| | AGAAGTAGGAATGGGGAGTGCTCCAGGAGTCAGGGTACT | |
| | CCCAGCTGACCTCCACCTGTGAGTTCTTCAAAGGCTTCAT | |
| | CTGCAAGTTCTTGGGCGGGTCTGGTTTGATGATGTCCCTG | |
| | ATGAAGAAGCTGGTGCTGTAGTTCTCATATTTATTCTGCT | |
| | GCCGTGCTTCCAACGCCAGTTCAATGGGCAGGGTCTCCTC | |
| | GGCAGTTGGGCAGGTGACATCCTCCTGGCAGGACACTGA | |
| | ATACTTCTCATAGTCCCTTTGGTCCAGTGTGACCTTCTCTG | |
| | CAGACAGAGACGCCATTCCACATGTCACTGCCCGAGAGT | |
| | CAGGGGAACTGCTACTGCTCTTGATGTTGAACTTCAAGTC | |
| | CATGTTTCTTTGCACCAGCCATGAGCACGTGAACCGTCCG | |
| | GAGTAATTTGGTGCTTCACACTTCAGGAAAGTCTTGTTTT | |
| | TGAAATTTTTTAAAATTTCAGTGGACCAAATTCCATTTTC | |
| | CTTCTTGTGGAGCAGCAGATGTGAGTGGCTCAGAGTCTCG | |
| | CCTCCTTTGTGGCAGGTGTACTGGCCAGCATCTAGAAACT | |
| | CTTTGACAGTGATGGTCAGGGTCTTTCCAGAGCCTATGAC | |
| | TCCATGTCTCTGGTCTGAGGTCCAGGTGATGTCATCTTCT | |
| | TCAGGCGTGTCACAGGTGAGGTTCACTGTTTCTCCAGGGG | |
| | CATCGGGAGTCCAGTCCACCTCTACAACATAAACGTCTTT | |
| | CTCCAGCTCCCACATGGCCATGAGTGGAGACACCAGCAA | |
| | AACGATGGCAAACCAGGAGATGGTTAGCTTCTGAGGACA | |
| | CATCTTGCTTTGCTGCGAGCTGCCTGGTCTGATGTGCCTG | |
| | GACACCCACTGTTCCTTCTGGTGTCgaattctttacgagggtaggaagtg | |
| | gtacggaaagttggtataagacaaaagtgttgtggaattgaagtttactcaaaaaatcagcactct | |
| | tttataggcgccctggtttacataagcaaagcttatacgttctctatcactgatagggagtaaactg | |
| | gatatacgttctctatcactgatagggagtaaactgtagatacgttctctatcactgatagggagta | |
| | aactggtcatacgttctctatcactgatagggagtaaactccttatacgttctctatcactgatagg | |
| | gagtaaagtctgcatacgttctctatcactgatagggagtaaactcttcatacgttctctatcactga | |
| | tagggagtaaactcgaggtgataattccacggggttggggttgcgccttttccaaggcagccct | |
| | gggtttgcgcagggacgcggctgctctgggcgtggttccgggaaacgcagcggcgccgacc | |
| | ctgggtctcgcacattcttcacgtccgttcgcagcgtcacccggatcttcgccgctacccttgtgg | |
| | gccccccggcgacgcttcctgctccgcccctaagtcgggaaggttccttgcggttcgcggcgt | |
| | gccggacgtgacaaacggaagccgcacgtctcactagtaccctcgcagacggacagcgcca | |
| | gggagcaatggcagcgcgccgaccgcgatgggctgtggccaatagcggctgctcagcagg | |
| | gcgcgccgagagcagcggccgggaaggggcggtgcgggaggcggggtgtggggcggta | |
| | gtgtgggccctgttcctgcccgcgcggtgttccgcattctgcaagcctccggagcgcacgtcg | |
| | gcagtcggctccctcgttgaccgaatcaccgacctctctccccagggggatcatcgaattacca | |
| | tgtctagactggacaagagcaaagtcataaactctgctctggaattactcaatggagtcggtatc | |
| | gaaggcctgacgacaaggaaactcgctcaaaagctgggagttgagcagcctaccctgtactg | |
| | gcacgtgaagaacaagcgggccctgctcgatgccctgccaatcgagatgctggacaggcatc | |
| | atacccactcctgccccctggaaggcgagtcatggcaagactttctgcggaacaacgccaagt | |
| | cataccgctgtgctctcctctcacatcgcgacggggctaaagtgcatcteggcaccegcccaac | |
| | agagaaacagtacgaaaccctggaaaatcagctcgcgttcctgtgtcagcaaggcttctccctg | |
| | gagaacgcactgtacgctctgtccgccgtgggccactttacactgggctgcgtattggaggaac | |
| | aggagcatcaagtagcaaaagaggaaagagagacacctaccaccgattctatgcccccacttc | |
| | tgaaacaagcaattgagctgttcgaccggcagggagccgaacctgccttccttttcggcctgga | |
| | actaatcatatgtggcctggagaaacagctaaagtgcgaaagcgggggccgaccgacgcc | |
| | cttgacgattttgacttagacatgctcccagccgatgcccttgacgactttgaccttgatatgctgc | |
| | ctgctgacgctcttgacgattttgaccttgacatgctccccgggtaaacgcgcgaatgtgtgtca | |
| | gttagggtgtggaaagtccccaggctccccagcaggcagaagtatgcaaagcatgcatctcaa | |
| | ttagtcagcaaccaggtgtggaaagtccccaggctccccagcaggcagaagtatgcaaagca | |
| | tgcatctcaattagtcagcaaccatagtcccgcccctaactccgcccatcccgcccctaactccg | |
| | cccagttccgcccattctccgccccatggctgactaattttttttatttatgcagaggccgaggccg | |
| | cctcggcctctgagctattccagaagtagtgaggaggcttttttggaggcctaggcttttgcaaaa | |
| | cgcgaccatgaccgagtacaagcccacggtgcgcctcgccacccgcgacgacgtcccccgg | |
| | gccgtacgcaccctcgccgccgegttcgccgactaccccgccacgcgccacacegtcgacc | |
| | cggaccgccacatcgagcgggtcaccgagctgcaagaactcttcctcacgcgcgtcgggctc | |
| | gacatcggcaaggtgtgggtcgcggacgacggcgccgcggtggcggtctggaccacgccg | |
| | gagagcgtcgaagcgggggcggtgttcgccgagatcggcccgcgcatggccgagttgagc | |
| | ggttcccggctggccgcgcagcaacagatggaaggcctcctggcgccgcaccggcccaag | |
| | gagcccgcgtggttcctggccaccgtcggcgtctcgcccgaccaccagggcaagggtctgg | |
| | gcagcgccgtcgtgctccccggagtggaggcggccgagcgcgccggggtgcccgccttcct | |
| | ggagacctccgcgccccgcaacctccccttctacgagcggctcggcttcaccgtcaccgccg | |
| | acgtcgaggtgcccgaaggaccgcgcacctggtgcatgacccgcaagcccggtgcctgaac | |
| | gcgtctggaacaatcaacctctggattacaaaatttgtgaaagattgactggtattcttaactatgtt | |
| | gctccttttacgctatgtggatacgctgctttaatgcctttgtatcatgctattgcttcccgtatggctt | |
| | tcattttctcctccttgtataaatcctggttgctgtctctttatgaggagttgtggcccgttgtcaggc | |
| | aacgtggcgtggtgtgcactgtgtttgctgacgcaacccccactggttggggcattgccaccac | |
| | ctgtcagctcctttccgggactttcgctttccccctccctattgccacggeggaactcategccgc | |
| | ctgccttgcccgctgctggacaggggctcggctgttgggcactgacaattccgtggtgttgtcg | |
| | gggaagctgacgtcctttccatggctgctcgcctgtgttgccacctggattctgcgcgggacgtc | |
| | cttctgctacgtcccttcggccctcaatccageggaccttccttcccgcggcctgctgccggctc | |
| | tgcggcctcttccgcgtcttcgccttcgccctcagacgagtcggatctccctttgggcegcctcc | |
| | ccgcctggaattaattctgcagtcgagacctagaaaaacatggagcaatcacaagtagcaatac | |
| | agcagctaccaatgctgattgtgcctggctagaagcacaagaggaggaggaggtgggttttcc | |
| | agtcacacctcaggtacctttaagaccaatgacttacaaggcagctgtagatcttagccacttttta | |
| | aaagaaaagaggggactggaagggctaattcactcccaacgaagacaagatatccttgatctg | |
| | tggatctaccacacacaaggctacttccctgattagcagaactacacaccagggccaggggtc | |
| | agatatccactgacctttggatggtgctacaagctagtaccagttgagccagataaggtagaag | |
| | aggccaataaaggagagaacaccagcttgttacaccctgtgagcctgcatgggatggatgacc | |
| | cggagagagaagtgttagagtggaggtttgacagccgcctagcatttcatcacgtggcccgag | |
| | agctgcatccggagtacttcaagaactgctgatatcgagcttgctacaagggactttccgctggg | |
| | gactttccagggaggcgtggcctggggggactggggagtggcgagccctcagatcctgcat | |
| | ataagcagctgctttttgcctgtactgggtctctctggttagaccagatctgagcctgggagctct | |
| | ctggctaactagggaacccactgcttaagcctcaataaagcttgccttgagtgcttcaagtagtgt | |
| | gtgcccgtctgttgtgtgactctggtaactagagatccctcagacccttttagtcagtgtggaaaat | |
| | ctctagcagtagtagttcatgtcatcttattattcagtatttataacttgcaaagaaatgaatatcaga | |
| | gagtgagaggccttgacattgctagcgtttaccgtcgacctctagctagagcttggcgtaatcat | |
| | ggtcatagctgtttcctgtgtgaaattgttatccgctcacaattccacacaacatacgagccggaa | |
| | gcataaagtgtaaagcctggggtgcctaatgagtgagctaactcacattaattgcgttgcgctca | |
| | ctgcccgctttccagtcgggaaacctgtcgtgccagctgcattaatgaatcggccaacgcgcg | |
| | gggagaggcggtttgcgtattgggcgctcttccgcttcctcgctcactgactcgctgcgctcggt | |
| | cgttcggctgcggcgagcggtatcagctcactcaaaggcggtaatacggttatccacagaatc | |
| | aggggataacgcaggaaagaacatgtgagcaaaaggccagcaaaaggccaggaaccgtaa | |
| | aaaggccgcgttgctggcgtttttccataggctccgcccccctgacgagcatcacaaaaatcga | |
| | cgctcaagtcagaggtggcgaaacccgacaggactataaagataccaggcgtttccccctgg | |
| | aagctccctcgtgcgctctcctgttccgaccctgccgcttaccggatacctgtccgcctttctccc | |
| | ttcgggaagcgtggcgctttctcatagctcacgctgtaggtatctcagttcggtgtaggtcgttcg | |
| | ctccaagctgggctgtgtgcacgaaccccccgttcagcccgaccgctgcgccttatccggtaa | |
| | ctatcgtcttgagtccaacccggtaagacacgacttatcgccactggcagcagccactggtaac | |
| | aggattagcagagcgaggtatgtaggcggtgctacagagttcttgaagtggtggcctaactacg | |
| | gctacactagaagaacagtatttggtatctgcgctctgctgaagccagttaccttcggaaaaaga | |
| | gttggtagctcttgatccggcaaacaaaccaccgctggtagcggtggtttttttgtttgcaagcag | |
| | cagattacgcgcagaaaaaaaggatctcaagaagatcctttgatcttttctacggggtctgacgc | |
| | tcagtggaacgaaaactcacgttaagggattttggtcatgagattatcaaaaaggatcttcaccta | |
| | gatccttttaaattaaaaatgaagttttaaatcaatctaaagtatatatgagtaaacttggtctgacag | |
| | ttaccaatgcttaatcagtgaggcacctatctcagcgatctgtctatttcgttcatccatagttgcct | |
| | gactccccgtcgtgtagataactacgatacgggagggcttaccatctggccccagtgctgcaat | |
| | gataccgcgagacccacgctcaccggctccagatttatcagcaataaaccagccagccggaa | |
| | gggccgagcgcagaagtggtcctgcaactttatccgcctccatccagtctattaattgttgccgg | |
| | gaagctagagtaagtagttcgccagttaatagtttgcgcaacgttgttgccattgctacaggcatc | |
| | gtggtgtcacgctcgtcgtttggtatggcttcattcagctccggttcccaacgatcaaggcgagtt | |
| | acatgatcccccatgttgtgcaaaaaagcggttagctccttcggtcctccgatcgttgtcagaagt | |
| | aagttggccgcagtgttatcactcatggttatggcagcactgcataattctcttactgtcatgccat | |
| | ccgtaagatgcttttctgtgactggtgagtactcaaccaagtcattctgagaatagtgtatgcggc | |
| | gaccgagttgctcttgcccggcgtcaatacgggataataccgcgccacatagcagaactttaaa | |
| | agtgctcatcattggaaaacgttcttcggggcgaaaactctcaaggatcttaccgctgttgagatc | |
| | cagttcgatgtaacccactcgtgcacccaactgatcttcagcatcttttactttcaccagcgtttctg | |
| | ggtgagcaaaaacaggaaggcaaaatgccgcaaaaaagggaataagggcgacacggaaat | |
| | gttgaatactcatactcttcctttttcaatattattgaagcatttatcagggttattgtctcatgagcgg | |
| | atacatatttgaatgtatttagaaaaataaacaaataggggttccgcgcacatttccccgaaaagt | |
| | gccacctgacgtcgacggatcgggagatcaacttgtttattgcagcttataatggttacaaataaa | |
| | gcaatagcatcacaaatttcacaaataaagcatttttttcactgcattctagttgtggtttgtccaaac | |
| | tcatcaatgtatottatcatgtctggatcaactggataactcaagctaaccaaaatcatcccaaact | |
| | tcccaccccataccctattaccactgccaattacctgtggtttcatttactctaaacctgtgattcctc | |
| | tgaattattttcattttaaagaaattgtatttgttaaatatgtactacaaacttagtagt | |
| 20 | gaattttaacaaaatattaacgcttacaatttaggtggcacttttcggggaaatgtgcgcggaacc | Human CD19t- |
| | cctatttgtttatttttctaaatacattcaaatatgtatccgctcatgagacaataaccctgataaatgc | PGK-NeoR-P2A- |
| | ttcaataatattgaaaaaggaagagtatgagtattcaacatttccgtgtcgcccttattcccttttttg | HSV TK |
| | cggcattttgccttcctgtttttgctcacccagaaacgctggtgaaagtaaaagatgctgaagatc | |
| | agttgggtgcacgagtgggttacatcgaactggatctcaacagcggtaagatccttgagagtttt | |
| | cgccccgaagaacgttttccaatgatgagcacttttaaagttctgctatgtggcgcggtattatcc | |
| | cgtattgacgccgggcaagagcaactcggtcgccgcatacactattctcagaatgacttggttg | |
| | agtactcaccagtcacagaaaagcatcttacggatggcatgacagtaagagaattatgcagtgc | |
| | tgccataaccatgagtgataacactgcggccaacttacttctgacaacgatcggaggaccgaa | |
| | ggagctaaccgcttttttgcacaacatgggggatcatgtaactcgccttgatcgttgggaaccgg | |
| | agctgaatgaagccataccaaacgacgagcgtgacaccacgatgcctgtagcaatggcaaca | |
| | acgttgcgcaaactattaactggcgaactacttactctagcttcccggcaacaattaatagactgg | |
| | atggaggcggataaagttgcaggaccacttctgcgctcggcccttccggctggctggtttattgc | |
| | tgataaatctggagccggtgagcgtgggtctcgcggtatcattgcagcactggggccagatgg | |
| | taagccctcccgtatcgtagttatctacacgacggggagtcaggcaactatggatgaacgaaat | |
| | agacagatcgctgagataggtgcctcactgattaagcattggtaactgtcagaccaagtttactc | |
| | atatatactttagattgatttaaaacttcatttttaatttaaaaggatctaggtgaagatcctttttgata | |
| | atctcatgaccaaaatcccttaacgtgagttttcgttccactgagcgtcagaccccgtagaaaag | |
| | atcaaaggatcttcttgagatcctttttttctgcgcgtaatctgctgcttgcaaacaaaaaaaccac | |
| | cgctaccagcggtggtttgtttgccggatcaagagctaccaactctttttccgaaggtaactggct | |
| | tcagcagagcgcagataccaaatactgttcttctagtgtagccgtagttaggccaccacttcaag | |
| | aactctgtagcaccgcctacatacctcgctctgctaatcctgttaccagtggctgctgccagtgg | |
| | cgataagtcgtgtcttaccgggttggactcaagacgatagttaccggataaggcgcagcggtc | |
| | gggctgaacggggggttcgtgcacacagcccagcttggagcgaacgacctacaccgaactg | |
| | agatacctacagcgtgagctatgagaaagcgccacgcttcccgaagggagaaaggcggaca | |
| | ggtatccggtaagcggcagggtcggaacaggagagcgcacgagggagcttccagggggaa | |
| | acgcctggtatctttatagtcctgtcgggtttcgccacctctgacttgagcgtcgatttttgtgatgc | |
| | tcgtcaggggggcggagcctatggaaaaacgccagcaacgcggcctttttacggttcctggcc | |
| | ttttgctggccttttgctcacatgttctttcctgcgttatcccctgattctgtggataaccgtattaccg | |
| | cctttgagtgagctgataccgctcgccgcagccgaacgaccgagcgcagcgagtcagtgagc | |
| | gaggaagcggaagagcgcccaatacgcaaaccgcctctccccgcgcgttggccgattcatta | |
| | atgcagctggcacgacaggtttcccgactggaaagcgggcagtgagcgcaacgcaattaatg | |
| | tgagttagctcactcattaggcaccccaggctttacactttatgcttccggctcgtatgttgtgtgg | |
| | aattgtgagcggataacaatttcacacaggaaacagctatgaccatgattacgccaagcgcgca | |
| | attaaccctcactaaagggaacaaaagctggagctgcaagcttaatgtagtcttatgcaatactct | |
| | tgtagtcttgcaacatggtaacgatgagttagcaacatgccttacaaggagagaaaaagcaccg | |
| | tgcatgccgattggtggaagtaaggtggtacgatcgtgccttattaggaaggcaacagacgggt | |
| | ctgacatggattggacgaaccactgaattgccgcattgcagagatattgtatttaagtgcctagct | |
| | cgatacataaacgggtctctctggttagaccagatctgagcctgggagctctctggctaactagg | |
| | gaacccactgcttaagcctcaataaagcttgccttgagtgcttcaagtagtgtgtgcccgtctgtt | |
| | gtgtgactctggtaactagagatccctcagacccttttagtcagtgtggaaaatctctagcagtgg | |
| | cgcccgaacagggacttgaaagcgaaagggaaaccagaggagctctctcgacgcaggactc | |
| | ggcttgctgaagcgcgcacggcaagaggcgaggggcggcgactggtgagtacgccaaaaa | |
| | ttttgactagcggaggctagaaggagagagatgggtgcgagagcgtcagtattaagcggggg | |
| | agaattagatcgcgatgggaaaaaattcggttaaggccagggggaaagaaaaaatataaatta | |
| | aaacatatagtatgggcaagcagggagctagaacgattcgcagttaatcctggcctgttagaaa | |
| | catcagaaggctgtagacaaatactgggacagctacaaccatcccttcagacaggatcagaag | |
| | aacttagatcattatataatacagtagcaaccctctattgtgtgcatcaaaggatagagataaaag | |
| | acaccaaggaagctttagacaagatagaggaagagcaaaacaaaagtaagaccaccgcaca | |
| | gcaagcggccgctgatcttcagacctggaggaggagatatgagggacaattggagaagtgaa | |
| | ttatataaatataaagtagtaaaaattgaaccattaggagtagcacccaccaaggcaaagagaa | |
| | gagtggtgcagagagaaaaaagagcagtgggaataggagctttgttccttgggttcttgggag | |
| | cagcaggaagcactatgggcgcagcgtcaatgacgctgacggtacaggccagacaattattgt | |
| | ctggtatagtgcagcagcagaacaatttgctgagggctattgaggcgcaacagcatctgttgca | |
| | actcacagtctggggcatcaagcagctccaggcaagaatcctggctgtggaaagatacctaaa | |
| | ggatcaacagctcctggggatttggggttgctctggaaaactcatttgcaccactgctgtgccttg | |
| | gaatgctagttggagtaataaatctctggaacagatttggaatcacacgacctggatggagtgg | |
| | gacagagaaattaacaattacacaagcttaatacactccttaattgaagaatcgcaaaaccagca | |
| | agaaaagaatgaacaagaattattggaattagataaatgggcaagtttgtggaattggtttaacat | |
| | aacaaattggctgtggtatataaaattattcataatgatagtaggaggcttggtaggtttaagaata | |
| | gtttttgctgtactttctatagtgaatagagttaggcagggatattcaccattatcgtttcagaccca | |
| | cctcccaaccccgaggggacccgacaggcccgaaggaatagaagaagaaggtggagaga | |
| | gagacagagacagatccattcgattagtgaacggatctcgacggtatcggttaacttttaaaaga | |
| | aaaggggggattggggggtacagtgcaggggaaagaatagtagacataatagcaacagaca | |
| | tacaaactaaagaattacaaaaacaaattacaaaattcaaaattttatcgataagcttgggagttcc | |
| | gcgttacataacttacggtaaatggcccgcctggctgaccgcccaacgacccccgcccattga | |
| | cgtcaataatgacgtatgttcccatagtaacgccaatagggactttccattgacgtcaatgggtgg | |
| | agtatttacggtaaactgcccacttggcagtacatcaagtgtatcatatgccaagtacgcccccta | |
| | ttgacgtcaatgacggtaaatggcccgcctggcattatgcccagtacatgaccttatgggactttc | |
| | ctacttggcagtacatctacgtattagtcatcgctattaccatggtgatgcggttttggcagtacatc | |
| | aatgggcgtggatagcggtttgactcacggggatttccaagtctccaccccattgacgtcaatg | |
| | ggagtttgttttggcaccaaaatcaacgggactttccaaaatgtcgtaacaactccgccccattga | |
| | cgcaaatgggcggtaggcgtgtacggtgggaggtctatataagcagagctcgtttagtgaacc | |
| | gtcagatcgcctggagacgccatccacgctgttttgacctccatagaagacaccgactctaga | |
| | GACACCAAGCTGGGTGCCCCGGAGAGTCTGACCACCATG | |
| | CCACCTCCTCGCCTCCTCTTCTTCCTCCTCTTCCTCACCCC | |
| | CATGGAAGTCAGGCCCGAGGAACCTCTAGTGGTGAAGGT | |
| | GGAAGAGGGAGATAACGCTGTGCTGCAGTGCCTCAAGGG | |
| | GACCTCAGATGGCCCCACTCAGCAGCTGACCTGGTCTCG | |
| | GGAGTCCCCGCTTAAACCCTTCTTAAAACTCAGCCTGGGC | |
| | CTGCCAGGCCTGGGAATCCACATGAGGCCCCTGGCCATC | |
| | TGGCTTTTCATCTTCAACGTCTCTCAACAGATGGGGGGCT | |
| | TCTACCTGTGCCAGCCAGGGCCCCCCTCTGAGAAGGCCTG | |
| | GCAGCCTGGCTGGACAGTCAATGTGGAGGGCAGCGGGGA | |
| | GCTGTTCCGGTGGAATGTTTCGGACCTAGGTGGCCTGGGC | |
| | TGTGGCCTGAAGAACAGGTCCTCAGAGGGCCCCAGCTCC | |
| | CCTTCCGGGAAGCTCATGAGCCCCAAGCTGTATGTGTGG | |
| | GCCAAAGACCGCCCTGAGATCTGGGAGGGAGAGCCTCCA | |
| | TGTGGCCCACCGAGGGACAGCCTGAACCAGAGCCTCAGC | |
| | CAGGACCTCACCATGGCCCCTGGCTCCACACTCTGGCTGT | |
| | CCTGTGGGGTACCCCCTGACTCTGTATCCAGGGGCCCCCT | |
| | CTCCTGGACCCATGTGCACCCCAAGGGGCCTAAGTCATTG | |
| | CTGAGCCTAGAGCTGAAGGACGATCGCCCGGCCAGAGAT | |
| | ATGTGGGTAATGGAGACGGGTCTGTTGTTGCCCCGGGCC | |
| | ACAGCTCAAGATGCTGGAAAGTATTATTGTCACCGTGGC | |
| | AACCTGACCATGTCATTCCACCTGGAGATCACTGCTCGGC | |
| | CAGTACTATGGCACTGGCTGCTGAGGACTGGTGGCTGGA | |
| | AGGTCTCAGCTGTGACTTTGGCTTATCTGATCTTCTGCggat | |
| | ccaccggtcgccaccatggtgagcaagggcgaggagctgttcaccggggtggtgcccatcct | |
| | ggtcgagctggacggcgacgtaaacggccacaagttcagcgtgtccggcgagggcgaggg | |
| | cgatgccacctacggcaagctgaccctgaagttcatctgcaccaccggcaagctgcccgtgcc | |
| | ctggcccaccctcgtgaccaccctgacctacggcgtgcagtgcttcagccgctaccccgacca | |
| | catgaagcagcacgacttcttcaagtccgccatgcccgaaggctacgtccaggagcgcaccat | |
| | cttcttcaaggacgacggcaactacaagacccgcgccgaggtgaagttcgagggcgacaccc | |
| | tggtgaaccgcatcgagctgaagggcatcgacttcaaggaggacggcaacatcctggggcac | |
| | aagctggagtacaactacaacagccacaacgtctatatcatggccgacaagcagaagaacgg | |
| | catcaaggtgaacttcaagatccgccacaacatcgaggacggcagcgtgcagctcgccgacc | |
| | actaccagcagaacacccccatcggcgacggccccgtgctgctgcccgacaaccactacctg | |
| | agcacccagtccgccctgagcaaagaccccaacgagaagcgcgatcacatggtcctgctgg | |
| | agttcgtgaccgccgccgggatcactctcggcatggacgagctgtacaagtaaagcggccgc | |
| | gtcgacaatcaacctctggattacaaaatttgtgaaagattgactggtattcttaactatgttgctcc | |
| | ttttacgctatgtggatacgctgctttaatgcctttgtatcatgctattgcttcccgtatggctttcatttt | |
| | ctcctccttgtataaatcctggttgctgtctctttatgaggagttgtggcccgttgtcaggcaacgtg | |
| | gcgtggtgtgcactgtgtttgctgacgcaacccccactggttggggcattgccaccacctgtca | |
| | gctcctttccgggactttcgctttccccctccctattgccacggeggaactcategccgcctgcct | |
| | tgcccgctgctggacaggggctcggctgttgggcactgacaattccgtggtgttgtcggggaa | |
| | gctgacgtcctttccatggctgctcgcctgtgttgccacctggattctgcgcgggacgtccttctg | |
| | ctacgtcccttcggccctcaatccagcggaccttccttcccgcggcctgctgccggctctgcgg | |
| | cctcttccgcgtcttcgccttcgccctcagacgagtcggatctccctttgggccgcctccccgcc | |
| | tggaattctgcagatatccggactagtgatctaattctaccgggtaggggaggcgcttttcccaa | |
| | ggcagtctggagcatgcgctttagcagccccgctgggcacttggcgctacacaagtggcctct | |
| | ggcctcgcacacattccacatccaccggtaggcgccaaccggctccgttctttggtggcccctt | |
| | cgcgccaccttctactcctcccctagtcaggaagttcccccccgccccgcagctcgcgtcgtgc | |
| | aggacgtgacaaatggaagtagcacgtctcactagtctcgtgcagatggacagcaccgctgag | |
| | caatggaagcgggtaggcctttggggcagcggccaatagcagctttgctccttcgctttctggg | |
| | ctcagaggctgggaaggggtgggtccgggggcgggctcaggggcgggctcaggggcggg | |
| | gcgggcgcccgaaggtcctccggaggcccggcattctgcacgcttcaaaagcgcacgtctgc | |
| | cgcgctgttctcctcttcctcatctccgggcctttcgacctgcagccaatatgggatcggccattg | |
| | aacaagatggattgcacgcaggttctccggccgcttgggtggagaggctattcggctatgactg | |
| | ggcacaacagacaatcggctgctctgatgccgccgtgttccggctgtcagcgcaggggcgcc | |
| | cggttctttttgtcaagaccgacctgtccggtgccctgaatgaactgcaggacgaggcagcgcg | |
| | gctatcgtggctggccacgacgggcgttccttgcgcagctgtgctcgacgttgtcactgaagcg | |
| | ggaagggactggctgctattgggcgaagtgccggggcaggatctcctgtcatctcaccttgctc | |
| | ctgccgagaaagtatccatcatggctgatgcaatgcggcggctgcatacgcttgatccggctac | |
| | ctgcccattcgaccaccaagcgaaacatcgcatcgagcgagcacgtactcggatggaagccg | |
| | gtcttgtcgatcaggatgatctggacgaagagcatcaggggctcgcgccagccgaactgttcg | |
| | ccaggctcaaggcgcgcatgcccgacggcgaggatctcgtcgtgacccatggcgatgcctgc | |
| | ttgccgaatatcatggtggaaaatggccgcttttctggattcatcgactgtggccggctgggtgtg | |
| | gcggaccgctatcaggacatagcgttggctacccgtgatattgctgaagagcttggcggcgaa | |
| | tgggctgaccgcttcctcgtgctttacggtatcgccgctcccgattcgcagcgcategccttctat | |
| | cgccttcttgacgagttcttctgaggggatccGCCACCAACTTCTCCCTGCTG | |
| | AAGCAGGCCGGCGACGTGGAGGAGAACCCCGGCCCCGCT | |
| | TCGTACCCCGGCCATCAACACGCGTCTGCGTTCGACCAGG | |
| | CTGCGCGTTCTCGCGGCCATAGCAACCGACGTACGGCGTT | |
| | GCGCCCTCGCCGGCAGCAAGAAGCCACGGAAGTCCGCCC | |
| | GGAGCAGAAAATGCCCACGCTACTGCGGGTTTATATAGA | |
| | CGGTCCCCACGGGATGGGGAAAACCACCACCACGCAACT | |
| | GCTGGTGGCCCTGGGTTCGCGCGACGATATCGTCTACGTA | |
| | CCCGAGCCGATGACTTACTGGCGGGTGCTGGGGGCTTCC | |
| | GAGACAATCGCGAACATCTACACCACACAACACCGCCTC | |
| | GACCAGGGTGAGATATCGGCCGGGGACGCGGCGGTGGTA | |
| | ATGACAAGCGCCCAGATAACAATGGGCATGCCTTATGCC | |
| | GTGACCGACGCCGTTCTGGCTCCTCATATCGGGGGGGAG | |
| | GCTGGGAGCTCACATGCCCCGCCCCCGGCCCTCACCCTCA | |
| | TCTTCGACCGCCATCCCATCGCCGCCCTCCTGTGCTACCC | |
| | GGCCGCGCGGTACCTTATGGGCAGCATGACCCCCCAGGC | |
| | CGTGCTGGCGTTCGTGGCCCTCATCCCGCCGACCTTGCCC | |
| | GGCACCAACATCGTGCTTGGGGCCCTTCCGGAGGACAGA | |
| | CACATCGACCGCCTGGCCAAACGCCAGCGCCCCGGCGAG | |
| | CGGCTGGACCTGGCTATGCTGGCTGCGATTCGCCGCGTTT | |
| | ACGGGCTACTTGCCAATACGGTGCGGTATCTGCAGTGCG | |
| | GCGGGTCGTGGCGGGAGGACTGGGGACAGCTTTCGGGGA | |
| | CGGCCGTGCCGCCCCAGGGTGCCGAGCCCCAGAGCAACG | |
| | CGGGCCCACGACCCCATATCGGGGACACGTTATTTACCCT | |
| | GTTTCGGGCCCCCGAGTTGCTGGCCCCCAACGGCGACCTG | |
| | TATAACGTGTTTGCCTGGGCCTTGGACGTCTTGGCCAAAC | |
| | GCCTCCGTTCCATGCACGTCTTTATCCTGGATTACGACCA | |
| | ATCGCCCGCCGGCTGCCGGGACGCCCTGCTGCAACTTACC | |
| | TCCGGGATGGTCCAGACCCACGTCACCACCCCCGGCTCC | |
| | ATACCGACGATATGCGACCTGGCGCGCACGTTTGCCCGG | |
| | GAGATGGGGGAGGCTAACTgggcccggtacctttaagaccaatgacttacaa | |
| | ggcagctgtagatcttagccactttttaaaagaaaaggggggactggaagggctagctcactcc | |
| | caacgaagacaagatctgctttttgcttgtactgggtctctctggttagaccagatctgagcctgg | |
| | gagctctctggctaactagggaacccactgcttaagcctcaataaagcttgccttgagtgcttca | |
| | agtagtgtgtgcccgtctgttgtgtgactctggtaactagagatccctcagacccttttagtcagtg | |
| | tggaaaatctctagcagtagtagttcatgtcatcttattattcagtatttataacttgcaaagaaatga | |
| | atatcagagagtgagaggaacttgtttattgcagcttataatggttacaaataaagcaatagcatc | |
| | acaaatttcacaaataaagcatttttttcactgcattctagttgtggtttgtccaaactcatcaatgtat | |
| | cttatcatgtctggctctagctatcccgcccctaactccgcccatccegcccctaactccgccca | |
| | gttccgcccattctccgccccatggctgactaattttttttatttatgcagaggccgaggccgcctc | |
| | ggcctctgagctattccagaagtagtgaggaggcttttttggaggcctagggacgtacccaattc | |
| | gccctatagtgagtcgtattacgcgcgctcactggccgtcgttttacaacgtcgtgactgggaaa | |
| | accctggcgttacccaacttaatcgccttgcagcacatccccctttcgccagctggcgtaatagc | |
| | gaagaggcccgcaccgatcgcccttcccaacagttgcgcagcctgaatggcgaatgggacg | |
| | cgccctgtagcggcgcattaagcgcggcgggtgtggtggttacgcgcagcgtgaccgctaca | |
| | cttgccagcgccctagcgcccgctcctttcgctttcttcccttcctttctcgccacgttcgccggctt | |
| | tccccgtcaagctctaaatcgggggctccctttagggttccgatttagtgctttacggcacctcga | |
| | ccccaaaaaacttgattagggtgatggttcacgtagtgggccatcgccctgatagacggtttttc | |
| | gccctttgacgttggagtccacgttctttaatagtggactcttgttccaaactggaacaacactcaa | |
| | ccctatctcggtctattcttttgatttataagggattttgccgatttcggcctattggttaaaaaatgag | |
| | ctgatttaacaaaaatttaacgc | |
| 21 | tggaagggctaattcactcccaaagaagacaagatatccttgatctgtggatctaccacacacaa | Tet Inducible |
| | ggctacttccctgattagcagaactacacaccagggccaggggtcagatatccactgacctttg | human Il-12 PuroR |
| | gatggtgctacaagctagtaccagttgagccagataaggtagaagaggccaataaaggagag | |
| | aacaccagcttgttacaccctgtgagcctgcatgggatggatgacccggagagagaagtgtta | |
| | gagtggaggtttgacagccgcctagcatttcatcacgtggcccgagagctgcatccggagtact | |
| | tcaagaactgctgatatcgagcttgctacaagggactttccgctggggactttccagggaggcg | |
| | tggcctgggcgggactggggagtggcgagccctcagatcctgcatataagcagctgctttttgc | |
| | ctgtactgggtctctctggttagaccagatctgagcctgggagctctctggctaactagggaacc | |
| | cactgcttaagcctcaataaagcttgccttgagtgcttcaagtagtgtgtgcccgtctgttgtgtga | |
| | ctctggtaactagagatccctcagacccttttagtcagtgtggaaaatctctagcagtggcgccc | |
| | gaacagggacttgaaagcgaaagggaaaccagaggagctctctcgacgcaggactcggctt | |
| | gctgaagcgcgcacggcaagaggcgaggggcggcgactggtgagtacgccaaaaattttga | |
| | ctagcggaggctagaaggagagagatgggtgcgagagcgtcagtattaagcgggggagaat | |
| | tagatcgcgatgggaaaaaattcggttaaggccagggggaaagaaaaaatataaattaaaaca | |
| | tatagtatgggcaagcagggagctagaacgattcgcagttaatcctggcctgttagaaacatca | |
| | gaaggctgtagacaaatactgggacagctacaaccatcccttcagacaggatcagaagaactt | |
| | agatcattatataatacagtagcaaccctctattgtgtgcatcaaaggatagagataaaagacac | |
| | caaggaagctttagacaagatagaggaagagcaaaacaaaagtaagaccaccgcacagcaa | |
| | gcggccggccgctgatcttcagacctggaggaggagatatgagggacaattggagaagtgaa | |
| | ttatataaatataaagtagtaaaaattgaaccattaggagtagcacccaccaaggcaaagagaa | |
| | gagtggtgcagagagaaaaaagagcagtgggaataggagctttgttccttgggttcttgggag | |
| | cagcaggaagcactatgggcgcagcgtcaatgacgctgacggtacaggccagacaattattgt | |
| | ctggtatagtgcagcagcagaacaatttgctgagggctattgaggcgcaacagcatctgttgca | |
| | actcacagtctggggcatcaagcagctccaggcaagaatcctggctgtggaaagatacctaaa | |
| | ggatcaacagctcctggggatttggggttgctctggaaaactcatttgcaccactgctgtgccttg | |
| | gaatgctagttggagtaataaatctctggaacagatttggaatcacacgacctggatggagtgg | |
| | gacagagaaattaacaattacacaagcttaatacactccttaattgaagaatcgcaaaaccagca | |
| | agaaaagaatgaacaagaattattggaattagataaatgggcaagtttgtggaattggtttaacat | |
| | aacaaattggctgtggtatataaaattattcataatgatagtaggaggcttggtaggtttaagaata | |
| | gtttttgctgtactttctatagtgaatagagttaggcagggatattcaccattatcgtttcagaccca | |
| | cctcccaaccccgaggggacccgacaggcccgaaggaatagaagaagaaggtggagaga | |
| | gagacagagacagatccattcgattagtgaacggatctcgacggtatcgcctttaaaagaaaag | |
| | gggggattggggggtacagtgcaggggaaagaatagtagacataatagcaacagacataca | |
| | aactaaagaattacaaaaacaaattacaaaaattcaaaattttcgggtttattacagggacagcag | |
| | agatccagtttatcgacttaacttgtttattgcagcttataatggttacaaataaggcaatagcatca | |
| | caaatttcacaaataaggcatttttttcactgcattctagttttggtttgtccaaactcatcaatgtatct | |
| | tatcatgtctggatctcaaatccctcggaagctgcgcctgtcttaggttggagtgatacatttttatc | |
| | acttttacccgtctttggattaggcagtagctctgacggccctcctgtcttaggttagtgaaaaatg | |
| | tcactctcttacccgtcattggctgtccagcttagctcgcaggggaggtggtctggatccGGA | |
| | AGCATTCAGATAGCTCATCACTCTATCAATAGTCACTGCC | |
| | CGAATTCTGAAAGCATGAAGAAGTATGCAGAGCTTGATT | |
| | TTAGTTTTATAAAAATCCGGTTCTTCAAGGGAGGATTTTT | |
| | GTGGCACAGTCTCACTGTTGAAATTCAGGGCCTGCATCAG | |
| | CTCATCAATAACTGCCAGCATGTTTTGATCTAGAAAGATC | |
| | TGCCTCTTAGGATCCATCAGAAGCTTTGCATTCATGGTCT | |
| | TGAACTCCACCTGGTACATCTTCAAGTCTTCATAAATACT | |
| | ACTAAGGCACAGGGCCATCATAAAAGAGGTCTTTCTGGA | |
| | GGCCAGGCAACTCCCATTAGTTATGAAAGAGGTCTCTCTG | |
| | GAATTTAGGCAACTCTCATTCTTGGTTAATTCCAATGGTA | |
| | AACAGGCCTCCACTGTGCTGGTTTTATCTTTTGTGATATCT | |
| | TCATGATCAATCTCTTCAGAAGTGCAAGGGTAAAATTCTA | |
| | GAGTTTGTCTGGCCTTCTGGAGCATGTTGCTGACGGCCCT | |
| | CAGCAGGTTTTGGGAGTGGTGAAGGCATGGGAACATTCC | |
| | TGGGTCTGGAGTGGCCACGGGGAGGTTTCTCCACCGCTA | |
| | GCGAGGCCTCCGCCAGAACCCCCGCCGCCACTCCCGCCG | |
| | CCGCCACTGCAGGGCACAGATGCCCATTCGCTCCAAGAT | |
| | GAGCTATAGTAGCGGTCCTGGGCCCGCACGCTAATGCTG | |
| | GCATTTTTGCGGCAGATGACCGTGGCTGAGGTCTTGTCCG | |
| | TGAAGACTCTATCTTTCTTTTCTCTCTTGCTCTTGCCCTGG | |
| | ACCTGAACGCAGAATGTCAGGGAGAAGTAGGAATGTGGA | |
| | GTACTCCAGGTGTCAGGGTACTCCCAGCTGACCTCCACCT | |
| | GCCGAGAATTCTTTAATGGCTTCAGCTGCAAGTTCTTGGG | |
| | TGGGTCAGGTTTGATGATGTCCCTGATGAAGAAGCTGCTG | |
| | GTGTAGTTTTCATACTTGAGCTTGTGAACGGCATCCACCA | |
| | TGACCTCAATGGGCAGACTCTCCTCAGCAGCTGGGCAGG | |
| | CACTGTCCTCCTGGCACTCCACTGAGTACTCATACTCCTT | |
| | GTTGTCCCCTCTGACTCTCTCTACAGAGAGTGTAGCAGCT | |
| | CCGCACGTCACCCCTTGGGGGTCAGAAGAGCCTCTGCTG | |
| | CTTTTGACACTGAATGTCAAATCAGTACTGATTGTCGTCA | |
| | GCCACCAGCAGGTGAAACGTCCAGAATAATTCTTGGCCT | |
| | CGCATCTTAGAAAGGTCTTATTTTTGGGTTCTTTCTGGTCC | |
| | TTTAAAATATCAGTGGACCAAATTCCATCTTCCTTTTTGT | |
| | GAAGCAGCAGGAGCGAATGGCTTAGAACCTCGCCTCCTT | |
| | TGTGACAGGTGTACTGGCCAGCATCTCCAAACTCTTTGAC | |
| | TTGGATGGTCAGGGTTTTGCCAGAGCCTAAGACCTCACTG | |
| | CTCTGGTCCAAGGTCCAGGTGATACCATCTTCTTCAGGGG | |
| | TGTCACAGGTGAGGACCACCATTTCTCCAGGGGCATCCG | |
| | GATACCAATCCAATTCTACGACATAAACATCTTTCTTCAG | |
| | TTCCCATATGGCCACGAGGGGAGATGCCAGAAAAACCAG | |
| | GGAAAACCAAGAGATGACCAACTGCTGGTGACACATCTT | |
| | GCTCTGGGCAGGACGGAGAGTCCAATGGCCCTGAAACAG | |
| | ATGTTGTTTCTTCTGGTGTCgaattctttacgagggtaggaagtggtacggaa | |
| | agttggtataagacaaaagtgttgtggaattgaagtttactcaaaaaatcagcactcttttataggc | |
| | gccctggtttacataagcaaagcttatacgttctctatcactgatagggagtaaactggatatacgt | |
| | tctctatcactgatagggagtaaactgtagatacgttctctatcactgatagggagtaaactggtc | |
| | atacgttctctatcactgatagggagtaaactccttatacgttctctatcactgatagggagtaaag | |
| | tctgcatacgttctctatcactgatagggagtaaactcttcatacgttctctatcactgatagggagt | |
| | aaactcgaggtgataattccacggggttggggttgcgccttttccaaggcagccctgggtttgc | |
| | gcagggacgcggctgctctgggcgtggttccgggaaacgcagcggcgccgaccctgggtct | |
| | cgcacattcttcacgtccgttcgcagcgtcacccggatcttcgccgctacccttgtgggcccccc | |
| | ggcgacgcttcctgctccgcccctaagtcgggaaggttccttgcggttcgcggcgtgccggac | |
| | gtgacaaacggaagccgcacgtctcactagtaccctcgcagacggacagcgccagggagca | |
| | atggcagcgcgccgaccgcgatgggctgtggccaatagcggctgctcagcagggcgcgcc | |
| | gagagcagcggccgggaaggggcggtgcgggaggcggggtgtggggcggtagtgtggg | |
| | ccctgttcctgcccgcgcggtgttccgcattctgcaagcctccggagcgcacgtcggcagtcg | |
| | gctccctcgttgaccgaatcaccgacctctctccccagggggatcatcgaattaccatgtctaga | |
| | ctggacaagagcaaagtcataaactctgctctggaattactcaatggagtcggtatcgaaggcc | |
| | tgacgacaaggaaactcgctcaaaagctgggagttgagcagcctaccctgtactggcacgtga | |
| | agaacaagcgggccctgctcgatgccctgccaatcgagatgctggacaggcatcatacccact | |
| | cctgccccctggaaggcgagtcatggcaagactttctgcggaacaacgccaagtcataccgct | |
| | gtgctctcctctcacatcgcgacggggctaaagtgcatctcggcacccgcccaacagagaaac | |
| | agtacgaaaccctggaaaatcagctcgcgttcctgtgtcagcaaggcttctccctggagaacgc | |
| | actgtacgctctgtccgccgtgggccactttacactgggctgcgtattggaggaacaggagcat | |
| | caagtagcaaaagaggaaagagagacacctaccaccgattctatgcccccacttctgaaacaa | |
| | gcaattgagctgttcgaccggcagggagccgaacctgccttccttttcggcctggaactaatcat | |
| | atgtggcctggagaaacagctaaagtgcgaaagcggcgggccgaccgacgcccttgacgatt | |
| | ttgacttagacatgctcccagccgatgcccttgacgactttgaccttgatatgctgcctgctgacg | |
| | ctcttgacgattttgaccttgacatgctccccgggtaaacgcgcgaatgtgtgtcagttagggtgt | |
| | ggaaagtccccaggctccccagcaggcagaagtatgcaaagcatgcatctcaattagtcagca | |
| | accaggtgtggaaagtccccaggctccccagcaggcagaagtatgcaaagcatgcatctcaat | |
| | tagtcagcaaccatagtcccgcccctaactcegcccatcccgcccctaactccgcccagttccg | |
| | cccattctccgccccatggctgactaattttttttatttatgcagaggccgaggccgcctcggcctc | |
| | tgagctattccagaagtagtgaggaggcttttttggaggcctaggcttttgcaaaacgcgaccat | |
| | gaccgagtacaagcccacggtgcgcctcgccacccgcgacgacgtcccccgggccgtacg | |
| | caccctcgccgccgcgttcgccgactaccccgccacgcgccacacegtcgacccggaccgc | |
| | cacatcgagcgggtcaccgagctgcaagaactcttcctcacgcgcgtcgggctcgacatcgg | |
| | caaggtgtgggtcgcggacgacggcgccgcggtggcggtctggaccacgccggagagcgt | |
| | cgaagcgggggcggtgttcgccgagatcggcccgcgcatggccgagttgagcggttcccgg | |
| | ctggccgcgcagcaacagatggaaggcctcctggcgccgcaccggcccaaggagcccgcg | |
| | tggttcctggccaccgtcggcgtctcgcccgaccaccagggcaagggtctgggcagcgccgt | |
| | cgtgctccccggagtggaggcggccgagcgcgccggggtgcccgccttcctggagacctcc | |
| | gcgccccgcaacctccccttctacgagcggctcggcttcaccgtcaccgccgacgtcgaggt | |
| | gcccgaaggaccgcgcacctggtgcatgacccgcaagcccggtgcctgaacgcgtctggaa | |
| | caatcaacctctggattacaaaatttgtgaaagattgactggtattcttaactatgttgctccttttac | |
| | gctatgtggatacgctgctttaatgcctttgtatcatgctattgcttcccgtatggctttcattttctcct | |
| | ccttgtataaatcctggttgctgtctctttatgaggagttgtggcccgttgtcaggcaacgtggcgt | |
| | ggtgtgcactgtgtttgctgacgcaacccccactggttggggcattgccaccacctgtcagctcc | |
| | tttccgggactttcgctttccccctccctattgccacggeggaactcategccgcctgccttgccc | |
| | gctgctggacaggggctcggctgttgggcactgacaattccgtggtgttgtcggggaagctga | |
| | cgtcctttccatggctgctcgcctgtgttgccacctggattctgcgcgggacgtccttctgctacg | |
| | tcccttcggccctcaatccagcggaccttccttcccgcggcctgctgccggctctgcggcctctt | |
| | ccgcgtcttcgccttcgccctcagacgagtcggatctccctttgggccgcctccccgcctggaa | |
| | ttaattctgcagtcgagacctagaaaaacatggagcaatcacaagtagcaatacagcagctacc | |
| | aatgctgattgtgcctggctagaagcacaagaggaggaggaggtgggttttccagtcacacct | |
| | caggtacctttaagaccaatgacttacaaggcagctgtagatcttagccactttttaaaagaaaag | |
| | aggggactggaagggctaattcactcccaacgaagacaagatatccttgatctgtggatctacc | |
| | acacacaaggctacttccctgattagcagaactacacaccagggccaggggtcagatatccac | |
| | tgacctttggatggtgctacaagctagtaccagttgagccagataaggtagaagaggccaataa | |
| | aggagagaacaccagcttgttacaccctgtgagcctgcatgggatggatgacccggagagag | |
| | aagtgttagagtggaggtttgacagccgcctagcatttcatcacgtggcccgagagctgcatcc | |
| | ggagtacttcaagaactgctgatatcgagcttgctacaagggactttccgctggggactttccag | |
| | ggaggcgtggcctgggcgggactggggagtggcgagccctcagatcctgcatataagcagc | |
| | tgctttttgcctgtactgggtctctctggttagaccagatctgagcctgggagctctctggctaact | |
| | agggaacccactgcttaagcctcaataaagcttgccttgagtgcttcaagtagtgtgtgcccgtct | |
| | gttgtgtgactctggtaactagagatccctcagacccttttagtcagtgtggaaaatctctagcagt | |
| | agtagttcatgtcatcttattattcagtatttataacttgcaaagaaatgaatatcagagagtgagag | |
| | gccttgacattgctagcgtttaccgtcgacctctagctagagcttggcgtaatcatggtcatagct | |
| | gtttcctgtgtgaaattgttatccgctcacaattccacacaacatacgagccggaagcataaagtg | |
| | taaagcctggggtgcctaatgagtgagctaactcacattaattgcgttgcgctcactgcccgcttt | |
| | ccagtcgggaaacctgtcgtgccagctgcattaatgaatcggccaacgcgcggggagaggc | |
| | ggtttgcgtattgggcgctcttccgcttcctcgctcactgactcgctgcgctcggtcgttcggctg | |
| | cggcgagcggtatcagctcactcaaaggcggtaatacggttatccacagaatcaggggataac | |
| | gcaggaaagaacatgtgagcaaaaggccagcaaaaggccaggaaccgtaaaaaggccgcg | |
| | ttgctggcgtttttccataggctccgcccccctgacgagcatcacaaaaatcgacgctcaagtca | |
| | gaggtggcgaaacccgacaggactataaagataccaggcgtttccccctggaagctccctcgt | |
| | gcgctctcctgttccgaccctgccgcttaccggatacctgtccgcctttctcccttcgggaagcgt | |
| | ggcgctttctcatagctcacgctgtaggtatctcagttcggtgtaggtcgttcgctccaagctggg | |
| | ctgtgtgcacgaaccccccgttcagcccgaccgctgcgccttatccggtaactatcgtcttgagt | |
| | ccaacccggtaagacacgacttatcgccactggcagcagccactggtaacaggattagcaga | |
| | gcgaggtatgtaggcggtgctacagagttcttgaagtggtggcctaactacggctacactagaa | |
| | gaacagtatttggtatctgcgctctgctgaagccagttaccttcggaaaaagagttggtagctctt | |
| | gatccggcaaacaaaccaccgctggtagcggtggtttttttgtttgcaagcagcagattacgcgc | |
| | agaaaaaaaggatctcaagaagatcctttgatcttttctacggggtctgacgctcagtggaacga | |
| | aaactcacgttaagggattttggtcatgagattatcaaaaaggatcttcacctagatccttttaaatt | |
| | aaaaatgaagttttaaatcaatctaaagtatatatgagtaaacttggtctgacagttaccaatgctta | |
| | atcagtgaggcacctatctcagcgatctgtctatttcgttcatccatagttgcctgactccccgtcg | |
| | tgtagataactacgatacgggagggcttaccatctggccccagtgctgcaatgataccgcgag | |
| | acccacgctcaccggctccagatttatcagcaataaaccagccagccggaagggccgagcgc | |
| | agaagtggtcctgcaactttatccgcctccatccagtctattaattgttgccgggaagctagagta | |
| | agtagttcgccagttaatagtttgcgcaacgttgttgccattgctacaggcatcgtggtgtcacgc | |
| | tcgtcgtttggtatggcttcattcagctccggttcccaacgatcaaggcgagttacatgatccccc | |
| | atgttgtgcaaaaaagcggttagctccttcggtcctccgatcgttgtcagaagtaagttggccgc | |
| | agtgttatcactcatggttatggcagcactgcataattctcttactgtcatgccatccgtaagatgct | |
| | tttctgtgactggtgagtactcaaccaagtcattctgagaatagtgtatgcggcgaccgagttgct | |
| | cttgcccggcgtcaatacgggataataccgcgccacatagcagaactttaaaagtgctcatcatt | |
| | ggaaaacgttcttcggggcgaaaactctcaaggatcttaccgctgttgagatccagttcgatgta | |
| | acccactcgtgcacccaactgatcttcagcatcttttactttcaccagcgtttctgggtgagcaaaa | |
| | acaggaaggcaaaatgccgcaaaaaagggaataagggcgacacggaaatgttgaatactcat | |
| | actcttcctttttcaatattattgaagcatttatcagggttattgtctcatgagcggatacatatttgaat | |
| | gtatttagaaaaataaacaaataggggttccgcgcacatttccccgaaaagtgccacctgacgt | |
| | cgacggatcgggagatcaacttgtttattgcagcttataatggttacaaataaagcaatagcatca | |
| | caaatttcacaaataaagcatttttttcactgcattctagttgtggtttgtccaaactcatcaatgtatc | |
| | ttatcatgtctggatcaactggataactcaagctaaccaaaatcatcccaaacttcccaccccata | |
| | ccctattaccactgccaattacctgtggtttcatttactctaaacctgtgattcctctgaattattttcat | |
| | tttaaagaaattgtatttgttaaatatgtactacaaacttagtagt | |

1. A method of generating patient-specific macrophages, comprising:
reprogramming one or more somatic cells of a sample from a subject to a pluripotent state;
culturing the one or more reprogrammed cells; and
differentiating the one or more reprogrammed cells to a macrophage-like phenotype, wherein the differentiation progresses without embryoid body formation.

2. The method of claim 1, wherein the reprogramming comprises delivering, to the one or more somatic cells, one or more expression vectors comprising one or more nucleic acids encoding OCT3/4, SOX2, KFL4, L-MYC, and LIN28.

3. The method of claim 2, further comprising delivering, to the one or more somatic cells, an expression vector comprising a nucleic acid encoding shRNA against p53.

4. (canceled)

5. The method of claim 1, wherein the culturing comprises expanding the one or more reprogrammed cells for about 10 passages to about 25 passages.

6. The method of claim 1, wherein the differentiation comprises removing b-FGF from a culture media and gradually adding IL-3 and M-CSF over a period of time.

7. The method of claim 6, wherein the IL-3 is added at a concentration of about 20 ng/ml to about 30 ng/ml, and wherein the M-CSF is added at a concentration of about 45 ng/ml to about 55 ng/ml.

8. (canceled)

9. The method of claim 6, wherein the period of time is about 15 days to about 20 days.

10. The method of claim 1, wherein the subject, has or is suspected of having a glioblastoma.

11. The method of claim 1, wherein the sample comprises pericranium from the subject or is enriched for periosteum-derived precursor cells (PDPCs).

12. (canceled)

13. The method of claim 1, further comprising, before reprogramming:
mincing the sample after sample collection;
culturing the minced sample for a period of time; and
isolating one or more somatic cells from the minced sample.

14. The method of claim 1, further comprising, after differentiating, delivering one or more treatment expression vectors to the one or more differentiated cells, the one or more treatment expression vectors comprising a therapeutic nucleic acid, each therapeutic nucleic acid independently encoding a cytokine, a checkpoint protein, or a self-destruction protein.

15. The method of claim 14, wherein the cytokine is interleukin-12 (IL-12), IFN-gamma, IL-15 or a combination thereof; wherein the checkpoint protein comprises one or more of PD-1/PD-L1 and CTLA-4/B7-1/B7-2; and wherein the self-destruction protein is a thymidine kinase.

16. (canceled)

17. (canceled)

18. (canceled)

19. The method of claim 14, wherein the nucleic acid encoding the cytokine is operably linked to an inducible promoter or wherein expression of the cytokine requires an inducing agent or removal of a repression element.

20. The method of claim 14, wherein the one or more treatment expression vectors comprise a nucleic acid encoding CD19.

21. (canceled)

22. A cell produced by the method of claim 1.

23. (canceled)

24. A pharmaceutical composition, comprising:
one or more cells of claim 22; and
a pharmaceutically acceptable excipient.

25. A method of treating a subject in need thereof, comprising: administering one or more differentiated cells to the subject wherein the one or more differentiated cells were produced by:
reprogramming one or more somatic cells of a sample from the subject to a pluripotent state;
culturing the one or more reprogrammed cells;
differentiating the one or more reprogrammed cells to produce a differentiated cell having a macrophage-like phenotype, wherein the differentiation progresses without embryoid body formation; and
incubating the one or more differentiated cells with one or more treatment expression vectors, wherein the one or more treatment expression vectors comprises one or more therapeutic nucleic acids encoding a cytokine, a checkpoint protein, or a self-destruction protein.

26. The method of claim 25, wherein the subject has or is suspected of having a glioblastoma, a neuroblastoma, or a melanoma.

27. (canceled)

28. The method of claim 25, wherein the administering is one or more differentiated cells are administered to the subject by intra-venous or intra-arterial injection.

29. (canceled)

30. The method of claim 25, further comprising administering ganciclovir (GCV) to the subject at a time point after administration of the one or more differentiated cells.


SEQUENCE LISTING (21 sequences)

SEQ ID NO: 1 - DNA, 36 bp, synthetic construct
  source 1..36: mol_type = other DNA
acgcagaagccgggtgccccagggagcctggctacc

SEQ ID NO: 2 - DNA, 54 bp, synthetic construct
  source 1..54: mol_type = other DNA
atgccatctcctctccctgtctccttcctcctctttcttaccttagtaggaggc

SEQ ID NO: 3 - DNA, 843 bp, synthetic construct
  source 1..843: mol_type = other DNA
aggccccagaagtccttactggtggaggtagaagagggaggcaatgttgtgctgccatgcctcccggactcctcacctgtctcttctgagaagctggcttggtatcgaggtaaccagtcaacacccttcctggagctgagccccgggtcccctggcctgggattgcacgtggggtccctgggcatcttgctagtgattgtcaatgtctcagaccatatggggggcttctacctgtgccagaagaggccccctttcaaggacatctggcagcctgcctggacagtgaacgtggaggatagtggggagatgttccggtggaatgcttcagacgtcagggacctggactgtgacctaaggaacaggtcctctgggagccacaggtccacttctggttcccagctgtatgtgtgggctaaagaccatcctaaggtctggggaacaaagcctgtatgtgcccctcgggggagcagtttgaatcagagtctaatcaaccaagatctcactgtggcacccggctccacactttggctgtcctgtggggtaccccctgtcccagtggccaaaggctccatctcctggacccatgtgcatcctaggagacctaatgtttcactactgagcctaagccttgggggagagcacccggtcagagagatgtgggtttgggggtctcttctgcttctgccccaagccacagctttagatgaaggcacctattattgtctccgaggaaacctgaccatcgagaggcacgtgaaggtcattgcaaggtcagtgtggctctggctgttgagaactggtggatggatagtcccagtggtgactttagtatatgtcatcttc

SEQ ID NO: 4 - DNA, 57 bp, synthetic construct
  source 1..57: mol_type = other DNA
gccaccaacttctccctgctgaagcaggccggcgacgtggaggagaaccccggcccc

SEQ ID NO: 5 - DNA, 1126 bp, synthetic construct
  source 1..1126: mol_type = other DNA
gcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaact

SEQ ID NO: 6 - DNA, 8798 bp, synthetic construct
  source 1..8798: mol_type = other DNA
gaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggactagtgatctaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgagggcccggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgc

SEQ ID NO: 7 - DNA, 66 bp, synthetic construct
  source 1..66: mol_type = other DNA
atgtgtcctcagaagctaaccatctcctggtttgccatcgttttgctggtgtctccactcatggcc

SEQ ID NO: 8 - DNA, 939 bp, Mus musculus
  source 1..939: mol_type = other DNA
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 9 - DNA, 53 bp, synthetic construct
  source 1..53: mol_type = other DNA
ggcggcggcgggagtggcggcgggggttctggcggaggcctcgctagcggtgg

SEQ ID NO: 10 - DNA, 579 bp, Mus musculus
  source 1..579: mol_type = other DNA
agggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 11 - DNA, 9180 bp, synthetic construct
  source 1..9180: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggtgtatacgggaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgaattaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaacgcgcgaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaacgcgaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 12 - DNA, 30 bp, synthetic construct
  source 1..30: mol_type = other DNA
aagctgggtgccccggagagtctgaccacc

SEQ ID NO: 13 - DNA, 60 bp, synthetic construct
  source 1..60: mol_type = other DNA
atgccacctcctcgcctcctcttcttcctcctcttcctcacccccatggaagtcaggccc

SEQ ID NO: 14 - DNA, 849 bp, synthetic construct
  source 1..849: mol_type = other DNA
gaggaacctctagtggtgaaggtggaagagggagataacgctgtgctgcagtgcctcaaggggacctcagatggccccactcagcagctgacctggtctcgggagtccccgcttaaacccttcttaaaactcagcctgggcctgccaggcctgggaatccacatgaggcccctggccatctggcttttcatcttcaacgtctctcaacagatggggggcttctacctgtgccagccagggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccatgtggcccaccgagggacagcctgaaccagagcctcagccaggacctcaccatggcccctggctccacactctggctgtcctgtggggtaccccctgactctgtatccaggggccccctctcctggacccatgtgcaccccaaggggcctaagtcattgctgagcctagagctgaaggacgatcgcccggccagagatatgtgggtaatggagacgggtctgttgttgccccgggccacagctcaagatgctggaaagtattattgtcaccgtggcaacctgaccatgtcattccacctggagatcactgctcggccagtactatggcactggctgctgaggactggtggctggaaggtctcagctgtgactttggcttatctgatcttctgc

SEQ ID NO: 15 - DNA, 56 bp, synthetic construct
  source 1..56: mol_type = other DNA
agaagaaacaacatctgtttcagggccattggactctccgtcctgcccagagcaag

SEQ ID NO: 16 - DNA, 918 bp, Homo sapiens
  source 1..918: mol_type = other DNA
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgtagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 17 - DNA, 591 bp, Homo sapiens
  source 1..591: mol_type = other DNA
agaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 18 - DNA, 6925 bp, synthetic construct
  source 1..6925: mol_type = other DNA
gagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagagacaccacgcagaagccgggtgccccagggagcctggctaccatgccatctcctctccctgtctccttcctcctctttcttaccttagtaggaggcaggccccagaagtccttactggtggaggtagaagagggaggcaatgttgtgctgccatgcctcccggactcctcacctgtctcttctgagaagctggcttggtatcgaggtaaccagtcaacacccttcctggagctgagccccgggtcccctggcctgggattgcacgtggggtccctgggcatcttgctagtgattgtcaatgtctcagaccatatggggggcttctacctgtgccagaagaggccccctttcaaggacatctggcagcctgcctggacagtgaacgtggaggatagtggggagatgttccggtggaatgcttcagacgtcagggacctggactgtgacctaaggaacaggtcctctgggagccacaggtccacttctggttcccagctgtatgtgtgggctaaagaccatcctaaggtctggggaacaaagcctgtatgtgcccctcgggggagcagtttgaatcagagtctaatcaaccaagatctcactgtggcacccggctccacactttggctgtcctgtggggtaccccctgtcccagtggccaaaggctccatctcctggacccatgtgcatcctaggagacctaatgtttcactactgagcctaagccttgggggagagcacccggtcagagagatgtgggtttgggggtctcttctgcttctgccccaagccacagctttagatgaaggcacctattattgtctccgaggaaacctgaccatcgagaggcacgtgaaggtcattgcaaggtcagtgtggctctggctgttgagaactggtggatggatagtcccagtggtgactttagtatatgtcatcttcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggactagtgatctaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgccaccaacttctccctgctgaagcaggccggcgacgtggaggagaaccccggccccgcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgggcccggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgc

SEQ ID NO: 19 - DNA, 10859 bp, synthetic construct
  source 1..10859: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccggcggagctcagatagcccatcaccctgttgatggtcacgacgcgggtgctgaaggcgtgaagcaggatgcagagcttcattttcactctgtaagggtctgcttctcccacaggaggtttctggcgcagagtctcgccattatgattcagagactgcatcagctcatcgatggccaccagcatgcccttgtctagaatgatctgctgatggttgtgattctgaagtgctgcgttgatggcctggaactctgtctggtacatcttcaagtcctcatagatgctaccaaggcacagggtcatcatcaaagacgtcttctgtgggggcaggcagctccctcttgttgtggaagaagtctctctagtagccaggcaactctcgttcttgtgtagttccagtggtaaacaggtcttcaatgtgctggtttggtcccgtgtgatgtcttcatgatcgatgtcttcagcagtgcaggaataatgtttcagtttttctctggccgtcttcaccatgtcatctgtggtcttcagcaggtttcgggactggctaagacacctggcaggtccagagactggaatgaccctccaccgctagcgaggcctccgccagaacccccgccgccactcccgccgccgccggatcggaccctgcagggaacacatgcccacttgctgcatgaggaattgtaatagcgatcctgagcttgcacgcagacattcccgcctttgcattggacttcggtagatgtcttctctacgaggaacgcacctttctggttacacccctcctctgtctccttcatcttttctttcttgcgctggattcgaacaaagaacttgagggagaagtaggaatggggagtgctccaggagtcagggtactcccagctgacctccacctgtgagttcttcaaaggcttcatctgcaagttcttgggcgggtctggtttgatgatgtccctgatgaagaagctggtgctgtagttctcatatttattctgctgccgtgcttccaacgccagttcaatgggcagggtctcctcggcagttgggcaggtgacatcctcctggcaggacactgaatacttctcatagtccctttggtccagtgtgaccttctctgcagacagagacgccattccacatgtcactgcccgagagtcaggggaactgctactgctcttgatgttgaacttcaagtccatgtttctttgcaccagccatgagcacgtgaaccgtccggagtaatttggtgcttcacacttcaggaaagtcttgtttttgaaattttttaaaatttcagtggaccaaattccattttccttcttgtggagcagcagatgtgagtggctcagagtctcgcctcctttgtggcaggtgtactggccagcatctagaaactctttgacagtgatggtcagggtctttccagagcctatgactccatgtctctggtctgaggtccaggtgatgtcatcttcttcaggcgtgtcacaggtgaggttcactgtttctccaggggcatcgggagtccagtccacctctacaacataaacgtctttctccagctcccacatggccatgagtggagacaccagcaaaacgatggcaaaccaggagatggttagcttctgaggacacatcttgctttgctgcgagctgcctggtctgatgtgcctggacacccactgttccttctggtgtcgaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgaattaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaacgcgcgaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaacgcgaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 20 - DNA, 10921 bp, synthetic construct
  source 1..10921: mol_type = other DNA
gaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagagacaccaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctgggcctgccaggcctgggaatccacatgaggcccctggccatctggcttttcatcttcaacgtctctcaacagatggggggcttctacctgtgccagccagggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccatgtggcccaccgagggacagcctgaaccagagcctcagccaggacctcaccatggcccctggctccacactctggctgtcctgtggggtaccccctgactctgtatccaggggccccctctcctggacccatgtgcaccccaaggggcctaagtcattgctgagcctagagctgaaggacgatcgcccggccagagatatgtgggtaatggagacgggtctgttgttgccccgggccacagctcaagatgctggaaagtattattgtcaccgtggcaacctgaccatgtcattccacctggagatcactgctcggccagtactatggcactggctgctgaggactggtggctggaaggtctcagctgtgactttggcttatctgatcttctgcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctgcagatatccggactagtgatctaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgccaccaacttctccctgctgaagcaggccggcgacgtggaggagaaccccggccccgcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgggcccggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgc

SEQ ID NO: 21 - DNA, 10850 bp, synthetic construct
  source 1..10850: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccggaagcattcagatagctcatcactctatcaatagtcactgcccgaattctgaaagcatgaagaagtatgcagagcttgattttagttttataaaaatccggttcttcaagggaggatttttgtggcacagtctcactgttgaaattcagggcctgcatcagctcatcaataactgccagcatgttttgatctagaaagatctgcctcttaggatccatcagaagctttgcattcatggtcttgaactccacctggtacatcttcaagtcttcataaatactactaaggcacagggccatcataaaagaggtctttctggaggccaggcaactcccattagttatgaaagaggtctctctggaatttaggcaactctcattcttggttaattccaatggtaaacaggcctccactgtgctggttttatcttttgtgatatcttcatgatcaatctcttcagaagtgcaagggtaaaattctagagtttgtctggccttctggagcatgttgctgacggccctcagcaggttttgggagtggtgaaggcatgggaacattcctgggtctggagtggccacggggaggtttctccaccgctagcgaggcctccgccagaacccccgccgccactcccgccgccgccactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctacagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatcttgctctgggcaggacggagagtccaatggccctgaaacagatgttgtttcttctggtgtcgaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgaattaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaacgcgcgaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaacgcgaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt